SEQUENCE LISTING (88 sequences)

SEQ ID NO: 1 - DNA, 5029 bp, polyomavirus
  misc_feature (1)..(5029): IPPyV
ccccttgcctccttctcttgttgcaacaaagagagaggctttggaggcttttccaaaactcattaaggtaagaagttctccacctatttgaaacttaattaacttgattctcaggtagtaagacttattttactttttgcagagccatggatcaaactctgtctttggaggagagaaatgagcttatggatttgctgcaacttacaagagcagcatggggaaatttatctcttatgaaaaaggcctacaaaacagtttccaaaatttaccaccctgataaaggtggtaatcctgagaaaatgcaaagattaaatgaattatttcaaaagctgcaagttaccttgctggaaataagaagtaattgtggatcctcctcttctcaggtagcttggtacttttgggatgagaattttagaacactaggtgcttttctaggagaaaaatttaatcaaagaattattggtggatatcctgattgcattacctataataagcccagttgttgctgcattgtatgtttattaaagcagcagcacaaaagtacaaaaataaataaaaaaaagccttgcttagtatggggtgaatgcttttgctacaagtgctacttactttggtttggatttcctgaggattttacatcttttaactactggaccttattaatgagaaatatggatttaagtttgctgcgcctctggactgagcttggattctaatgtaagtatttttttttttagggatactacagtgattctccatactttactgaaactccattttcctactgtgaaagaaaaaatgaagatccagaaggggggtcatggggtaaatggtggagggagtttgttaacaaagaatatgatgatttattttgttcagagactatatcatctagtgatgatgaaaataatcctggaccttcagcccctccaccctcttctgcttctgcctctgaagaccctgatcctgaggaagaagctggatcatcccagagttcatttacctgcacgccccccaaaagaaagaagcctgaacctaatactccagaggactttcctatgtgtttgtattcttttttaagtcatgctatctatagtaataagactatgaattgctttttaatttatactacagttgaaaaaagtaagcagctttacagaactgttgaaaaatcaaaaattaaagtagattttaaggctattttcttgtataaagatgatggaattgaaggaggcctgttatattttattaccttaggcaagcatagagtttctgctgtaaaacatttttgtgttgcacagtgtacttttagtttcattcattgtaaagctgttattaaacctttagagctttatagagctttaggtaaacccccttttaagttgttggaagaaaacaagcctggtgtatccatgtttgactttcaggaggaaaaagaacaggctgtaaattggcaggagatttgtaattatgcggttgaagcaaaaattactgatgtattactgttgcttggcatctacctagattttgcagtggaacctggcacttgctctaaatgtgaaaaaaagagtcataaattccattataattaccacagtaagcatcatgccaatgcttgccttttcttggagagtaaatcccaaaagaatatctgccaacaagcagttgaccaggtccttgcagctaagagattaaagcttgtagagtgtactagaatggagcttttagaagatagatttattcaactttttgatgaaatggaggactttttgcatggggaaattgaaattctaagatggatgtcaggtgtggcctggtacaccattttacttgacaactcatgggatgtttttcaaaaaattttgcagctggtaaccacaagccaaccaaaaaagaggaacatactctttaaaggccctataaatagtggcaaaaccaccttagcttctgctttcatgcacttttttgatggtaaagctttgaatattaactgcccagcagaaaaattatcttttgagttaggctgtgctattgatcaattttgtgttttgcttgatgatgtgaagggccagataactttaaacaaaacatcttcaacctgggcagggagttaataatttggataatttaagagatcacttagatggtactattaaagttaatctggaaaaaaaacatgtgaataaaagaagccagatatttcctcctgtaattatgacaatgaatgagtatttactcccacccacagtgggagttagatttgctttgcacatccattttcattgtaagacctacctgaagcagagtttagaaaaaagtgacttaattgaaaaaagaattcttaactctggttacacaatcttgctgttacttttatggtacaaccctgttgattcttttactccaaaagtccaggaatatgttgtaaaatggaaagagattttagagagacatgtatctattactcagtttggaaatatccagcaaaacattcttgatggtaaagatcccctgcatggaattgttattgaagaacaagcataacaatttgtacttttcttcttcagatttattcatggttttgttaatacaataaagctttacaatgcaaccagcctcattgatcatttgagcgaggggggacagtttgattctgacaaaatttatcaacatatctatcaagatcagggtccccgggtaggccttcagtgccctgataaattctgacttcttccacttgccctgaagttccttccatgggttgtccctgaatttgaggcataagaccagagaacaagctattaaggagagagcttacaggataaggatttttaacaacccgtttccttagagttacattaaaatatctaggtaggcctctccagttttgggattctgaataattagtgtgaattccaacaatatcaacagcagacaaaaacagcttgtcccctttacaaagaggccccactccattttcatccaatagcaccgtggttacagagttagtaaactgcataacagggggggttgtggctcctccagtgaagctcccatagtatcttgtattctcatttttagaggggtctgggctccaaacttctacagggtactttccatctttgtctagcaaagctttagcattaggatccaaggcctggtttactggtttcatatttttaatagttaccatatcagtaggataggtagtagtagagctagcaactaggccttgaagttcaagaggctctccacccactgaaaacatgtggagagtggtaccctgcactgggatagttcctgaggatgagccatagatatatttacccccctgatgcagatttactagagaactaatccccatgacttcagtttttactgacacagcctcccacattagaagtgtgtcacaggtcatgtcttcatttagcatgggtagcttaattacagccactgagtaagtagggagggtagtagcattagggttatcactagccttgctggaggcaacatttatatctgcactgtagccatacaattcatcagtagggttattgtttcccattcttggatttaggtaggcctcaatttgagttatagcatctgggcctgttctaacttctagaacttctacccctcctttgacaaggagtttgggaacgggagcgggtgttggacaggttttttttacaggacatgctccgcactcttgcctttttctttggggggccatattcttctttttccaatttactcagctctttctcccaggttggagttatatcaccatacaaacctagaattagaggaagcatccaatcttgagatactctttgatttgcccctcctggagcagcataatgttgtacatagtgtccagatctaggctctgaaccccctatctcccgcctcagcctaacttgttcctgatattcaaattgttccctagttgggggtagctctcctctacttctatactgttgtctcagttgtataggatttactgaaggcaaacctctataatattcttcaacactagaataaatatgaataggcccattagttagggcccaccttgcattttctgcaatttgagcaagcatatgttgaaattcattagtaccccttacagccaaagctctagaagtatatccaatttgctgagtagcttgcctcataatatttctccatatttccctgccaacagcatgaaacagtgattctccccagtctagagcagcattcagatagtaactaaaagaagacaagcctggaaacagatagtccacttgaggaaaccaaggagtgagagccatactaacaagtggtacttctttggaatatccaaaagttgttactcctgcagcaaccacagcactggcacctgtaacagtttgaaaaaagattcctattcctatggcattgttcagggcagttggtagagcactaagtagagaaaattcttctgctgacagtccagtcaaagttaaggcctctaaggctcctaggcctgctacatctacagcttctatttcaattaaccaagctgcttcagtagtagcagcagctatagcttccccagacaatattgcatcaacagtaaatccagtacttaaacttaattgttcagccagttcaaaaatatccaacaatatagttaaaaccccccccatatcttacccaagttcaaaattggcgcgtttagtttaggcgggcttcttctctgttgaccttagatggcaaaatgatttcacttcccccttagctactggtatctagaggggttgctaagcgccacctagcaacggaaaccgcaatgcacttgtttgtcacacaccggaatgtcaccaaaaagtccccaggcagtcaagtggagcaggtagataccacagataccacagtttcctctagagttgattctgcaaaaataacctgtcatttagttcctcaatttgcaaagttatctccctgtcactcaaaatcactatggcaacacaggtttttttttgatagtataagaggccagggg

SEQ ID NO: 2 - DNA, 1116 bp, polyomavirus
  CDS (1)..(1116)
  misc_feature (1)..(371): VP1
atggccccccaaagaaaaaggcaagagtgcggagcatgtcctgtaaaaaaaacctgtccaacacccgctcccgttcccaaactccttgtcaaaggaggggtagaagttctagaagttagaacaggcccagatgctataactcaaattgaggcctacctaaatccaagaatgggaaacaataaccctactgatgaattgtatggctacagtgcagatataaatgttgcctccagcaaggctagtgataaccctaatgctactaccctccctacttactcagtggctgtaattaagctacccatgctaaatgaagacatgacctgtgacacacttctaatgtgggaggctgtgtcagtaaaaactgaagtcatggggattagttctctagtaaatctgcatcaggggggtaaatatatctatggctcatcctcaggaactatcccagtgcagggtaccactctccacatgttttcagtgggtggagagcctcttgaacttcaaggcctagttgctagctctactactacctatcctactgatatggtaactattaaaaatatgaaaccagtaaaccaggccttggatcctaatgctaaagctttgctagacaaagatggaaagtaccctgtagaagtttggagcccagacccctctaaaaatgagaatacaagatactatgggagcttcactggaggagccacaaccccccctgttatgcagtttactaactctgtaaccacggtgctattggatgaaaatggagtggggcctctttgtaaaggggacaagctgtttttgtctgctgttgatattgttggaattcacactaattattcagaatcccaaaactggagaggcctacctagatattttaatgtaactctaaggaaacgggttgttaaaaatccttatcctgtaagctctctccttaatagcttgttctctggtcttatgcctcaaattcagggacaacccatggaaggaacttcagggcaagtggaagaagtcagaatttatcagggcactgaaggcctacccggggaccctgatcttgatagatatgttgataaattttgtcagaatcaaactgtcccccctcgctcaaatgatcaatga

SEQ ID NO: 3 - protein, 371 aa, polyomavirus
MAPQRKRQECGACPVKKTCPTPAPVPKLLVKGGVEVLEVRTGPDAITQIEAYLNPRMGNNNPTDELYGYSADINVASSKASDNPNATTLPTYSVAVIKLPMLNEDMTCDTLLMWEAVSVKTEVMGISSLVNLHQGGKYIYGSSSGTIPVQGTTLHMFSVGGEPLELQGLVASSTTTYPTDMVTIKNMKPVNQALDPNAKALLDKDGKYPVEVWSPDPSKNENTRYYGSFTGGATTPPVMQFTNSVTTVLLDENGVGPLCKGDKLFLSAVDIVGIHTNYSESQNWRGLPRYFNVTLRKRVVKNPYPVSSLLNSLFSGLMPQIQGQPMEGTSGQVEEVRIYQGTEGLPGDPDLDRYVDKFCQNQTVPPRSNDQ

SEQ ID NO: 4 - DNA, 1059 bp, polyomavirus
  CDS (1)..(1059)
  misc_feature (1)..(352): VP2
atggggggggttttaactatattgttggatatttttgaactggctgaacaattaagtttaagtactggatttactgttgatgcaatattgtctggggaagctatagctgctgctactactgaagcagcttggttaattgaaatagaagctgtagatgtagcaggcctaggagccttagaggccttaactttgactggactgtcagcagaagaattttctctacttagtgctctaccaactgccctgaacaatgccataggaataggaatcttttttcaaactgttacaggtgccagtgctgtggttgctgcaggagtaacaacttttggatattccaaagaagtaccacttgttagtatggctctcactccttggtttcctcaagtggactatctgtttccaggcttgtcttcttttagttactatctgaatgctgctctagactggggagaatcactgtttcatgctgttggcagggaaatatggagaaatattatgaggcaagctactcagcaaattggatatacttctagagctttggctgtaaggggtactaatgaatttcaacatatgcttgctcaaattgcagaaaatgcaaggtgggccctaactaatgggcctattcatatttattctagtgttgaagaatattatagaggtttgccttcagtaaatcctatacaactgagacaacagtatagaagtagaggagagctacccccaactagggaacaatttgaatatcaggaacaagttaggctgaggcgggagatagggggttcagagcctagatctggacactatgtacaacattatgctgctccaggaggggcaaatcaaagagtatctcaagattggatgcttcctctaattctaggtttgtatggtgatataactccaacctgggagaaagagctgagtaaattggaaaaagaagaatatggccccccaaagaaaaaggcaagagtgcggagcatgtcctgtaaaaaaaacctgtccaacacccgctcccgttcccaaactccttgtcaaaggaggggtagaagttctagaagttag

SEQ ID NO: 5 - protein, 352 aa, polyomavirus
MGGVLTILLDIFELAEQLSLSTGFTVDAILSGEAIAAATTEAAWLIEIEAVDVAGLGALEALTLTGLSAEEFSLLSALPTALNNAIGIGIFFQTVTGASAVVAAGVTTFGYSKEVPLVSMALTPWFPQVDYLFPGLSSFSYYLNAALDWGESLFHAVGREIWRNIMRQATQQIGYTSRALAVRGTNEFQHMLAQIAENARWALTNGPIHIYSSVEEYYRGLPSVNPIQLRQQYRSRGELPPTREQFEYQEQVRLRREIGGSEPRSGHYVQHYAAPGGANQRVSQDWMLPLILGLYGDITPTWEKELSKLEKEEYGPPKKKARVRSMSCKKNLSNTRSRSQTPCQRRGRSSRS

SEQ ID NO: 6 - DNA, 702 bp, polyomavirus
  CDS (1)..(702)
  misc_feature (1)..(702): VP3
atggctctcactccttggtttcctcaagtggactatctgtttccaggcttgtcttcttttagttactatctgaatgctgctctagactggggagaatcactgtttcatgctgttggcagggaaatatggagaaatattatgaggcaagctactcagcaaattggatatacttctagagctttggctgtaaggggtactaatgaatttcaacatatgcttgctcaaattgcagaaaatgcaaggtgggccctaactaatgggcctattcatatttattctagtgttgaagaatattatagaggtttgccttcagtaaatcctatacaactgagacaacagtatagaagtagaggagagctacccccaactagggaacaatttgaatatcaggaacaagttaggctgaggcgggagatagggggttcagagcctagatctggacactatgtacaacattatgctgctccaggaggggcaaatcaaagagtatctcaagattggatgcttcctctaattctaggtttgtatggtgatataactccaacctgggagaaagagctgagtaaattggaaaaagaagaatatggccccccaaagaaaaaggcaagagtgcggagcatgtcctgtaaaaaaaacctgtccaacacccgctcccgttcccaaactccttgtcaaaggaggggtagaagttctagaagttag

SEQ ID NO: 7 - protein, 233 aa, polyomavirus
MALTPWFPQVDYLFPGLSSFSYYLNAALDWGESLFHAVGREIWRNIMRQATQQIGYTSRALAVRGTNEFQHMLAQIAENARWALTNGPIHIYSSVEEYYRGLPSVNPIQLRQQYRSRGELPPTREQFEYQEQVRLRREIGGSEPRSGHYVQHYAAPGGANQRVSQDWMLPLILGLYGDITPTWEKELSKLEKEEYGPPKKKARVRSMSCKKNLSNTRSRSQTPCQRRGRSSRS

SEQ ID NO: 8 - DNA, 1614 bp, polyomavirus
  CDS (1)..(1614)
  misc_feature (1)..(1614): nt genomic Large T polyomavirus
atggatcaaactctgtctttggaggagagaaatgagcttatggatttgctgcaacttacaagagcagcatggggaaatttatctcttatgaaaaaggcctacaaaacagtttccaaaatttaccaccctgataaaggtggtaatcctgagaaaatgcaaagattaaatgaattatttcaaaagctgcaagttaccttgctggaaataagaagtaattgtggatcctcctcttctcagggatactacagtgattctccatactttactgaaactccattttcctactgtgaaagaaaaaatgaagatccagaaggggggtcatggggtaaatggtggagggagtttgttaacaaagaatatgatgatttattttgttcagagactatatcatctagtgatgatgaaaataatcctggaccttcagcccctccaccctcttctgcttctgcctctgaagaccctgatcctgaggaagaagctggatcatcccagagttcatttacctgcacgccccccaaaagaaagaagcctgaacctaatactccagaggactttcctatgtgtttgtattcttttttaagtcatgctatctatagtaataagactatgaattgctttttaatttatactacagttgaaaaaagtaagcagctttacagaactgttgaaaaatcaaaaattaaagtagattttaaggctattttcttgtataaagatgatggaattgaaggaggcctgttatattttattaccttaggcaagcatagagtttctgctgtaaaacatttttgtgttgcacagtgtacttttagtttcattcattgtaaagctgttattaaacctttagagctttatagagctttaggtaaacccccttttaagttgttggaagaaaacaagcctggtgtatccatgtttgactttcaggaggaaaaagaacaggctgtaaattggcaggagatttgtaattatgcggttgaagcaaaaattactgatgtattactgttgcttggcatctacctagattttgcagtggaacctggcacttgctctaaatgtgaaaaaaagagtcataaattccattataattaccacagtaagcatcatgccaatgcttgccttttcttggagagtaaatcccaaaagaatatctgccaacaagcagttgaccaggtccttgcagctaagagattaaagcttgtagagtgtactagaatggagcttttagaagatagatttattcaactttttgatgaaatggaggactttttgcatggggaaattgaaattctaagatggatgtcaggtgtggcctggtacaccattttacttgacaactcatgggatgtttttcaaaaaattttgcagctggtaaccacaagccaaccaaaaaagaggaacatactctttaaaggccctataaatagtggcaaaaccaccttagcttctgctttcatgcacttttttgatggtaaagctttgaatattaactgcccagcagaaaaattatcttttgagttaggctgtgctattgatcaattttgtgttttgcttgatgatgtgaagggccagataactttaaacaaaacatcttcaacctgggcagggagttaa

SEQ ID NO: 9 - protein, 537 aa, polyomavirus
MDQTLSLEERNELMDLLQLTRAAWGNLSLMKKAYKTVSKIYHPDKGGNPEKMQRLNELFQKLQVTLLEIRSNCGSSSSQGYYSDSPYFTETPFSYCERKNEDPEGGSWGKWWREFVNKEYDDLFCSETISSSDDENNPGPSAPPPSSASASEDPDPEEEAGSSQSSFTCTPPKRKKPEPNTPEDFPMCLYSFLSHAIYSNKTMNCFLIYTTVEKSKQLYRTVEKSKIKVDFKAIFLYKDDGIEGGLLYFITLGKHRVSAVKHFCVAQCTFSFIHCKAVIKPLELYRALGKPPFKLLEENKPGVSMFDFQEEKEQAVNWQEICNYAVEAKITDVLLLLGIYLDFAVEPGTCSKCEKKSHKFHYNYHSKHHANACLFLESKSQKNICQQAVDQVLAAKRLKLVECTRMELLEDRFIQLFDEMEDFLHGEIEILRWMSGVAWYTILLDNSWDVFQKILQLVTTSQPKKRNILFKGPINSGKTTLASAFMHFFDGKALNINCPAEKLSFELGCAIDQFCVLLDDVKGQITLNKTSSTWAGS

SEQ ID NO: 10 - DNA, 570 bp, polyomavirus
  CDS (1)..(570)
  misc_feature (1)..(570): nt -  genomic DNA  - Small T polyomavirus
atggatcaaactctgtctttggaggagagaaatgagcttatggatttgctgcaacttacaagagcagcatggggaaatttatctcttatgaaaaaggcctacaaaacagtttccaaaatttaccaccctgataaaggtggtaatcctgagaaaatgcaaagattaaatgaattatttcaaaagctgcaagttaccttgctggaaataagaagtaattgtggatcctcctcttctcaggtagcttggtacttttgggatgagaattttagaacactaggtgcttttctaggagaaaaatttaatcaaagaattattggtggatatcctgattgcattacctataataagcccagttgttgctgcattgtatgtttattaaagcagcagcacaaaagtacaaaaataaataaaaaaaagccttgcttagtatggggtgaatgcttttgctacaagtgctacttactttggtttggatttcctgaggattttacatcttttaactactggaccttattaatgagaaatatggatttaagtttgctgcgcctctggactgagcttggattctaa

SEQ ID NO: 11 - protein, 189 aa, polyomavirus
MDQTLSLEERNELMDLLQLTRAAWGNLSLMKKAYKTVSKIYHPDKGGNPEKMQRLNELFQKLQVTLLEIRSNCGSSSSQVAWYFWDENFRTLGAFLGEKFNQRIIGGYPDCITYNKPSCCCIVCLLKQQHKSTKINKKKPCLVWGECFCYKCYLLWFGFPEDFTSFNYWTLLMRNMDLSLLRLWTELGF

SEQ ID NO: 12 - DNA, 57 bp, polyomavirus
ttgtatggctacagtgcagatataaatgttgcctccagcaaggctagtgataaccct

SEQ ID NO: 13 - protein, 19 aa, polyomavirus
LYGYSADINVASSKASDNP

SEQ ID NO: 14 - DNA, 54 bp, polyomavirus
ctgcatcaggggggtaaatatatctatggctcatcctcaggaactatcccagtg

SEQ ID NO: 15 - protein, 18 aa, polyomavirus
LHQGGKYIYGSSSGTIPV

SEQ ID NO: 16 - DNA, 15 bp, polyomavirus
aattattcagaatcc

SEQ ID NO: 17 - protein, 5 aa, polyomavirus
NYSES

SEQ ID NO: 18 - DNA, 244 bp, polyomavirus
  misc_feature (1)..(244): contig6449 IPPyV
cttcacatcatcaagcaaaacacaaaattgatcaatagcacagcctaactcaaaagataatttttctgctgggcagttaatattcaaagctttaccatcaaaaaagtgcatgaaagcagaagctaaggtggttttgccactatttatagggcctttaaagagtatgttcctcttttttggttggcttgtggttaccagctgcaaaattttttgaaaaacatcccatgagttgtcaagtaaaatg

SEQ ID NO: 19 - DNA, 21 bp, polyomavirus
tgatcaatagcacagcctaac

SEQ ID NO: 20 - DNA, 21 bp, polyomavirus
aattttgcagctggtaaccac

SEQ ID NO: 21 - DNA, 190 bp, polyomavirus
  misc_feature (1)..(190): contig 650
aatccatatttctcattaataaggtccagtagttaaaagatgtaaaatcctcaggaaatccaaaccaaagtaagtagcacttgtagcaaaagcattcaccccatactaagcaaggcttttttttatttatttttgtacttttgtgctgctgctttaataaacatacaatgcagcaacaactgggcttatt

SEQ ID NO: 22 - DNA, 22 bp, polyomavirus
tctcattaataaggtccagtag

SEQ ID NO: 23 - DNA, 22 bp, polyomavirus
tattaaagcagcagcacaaaag

SEQ ID NO: 24 - DNA, 329 bp, polyomavirus
  misc_feature (1)..(329): contig 816
aagtaaaatggtgtaccaggccacacctgacatccatcttagaatttcaatttccccatgcaaaaagtcctccatttcatcaaaaagttgaataaatctatcttctaaaagctccattctagtacactctacaagctttaatctcttagctgcaaggacctggtcaactgcttgttggcagatattcttttgggatttactctccaagaaaaggcaagcattggcatgatgcttactgtggtaattataatggaatttatgactctttttttcacatttagagcaagtgccaggttccactgcaaaatctaggtagatgccaagcaaca

SEQ ID NO: 25 - DNA, 21 bp, polyomavirus
aaatggtgtaccaggccacac

SEQ ID NO: 26 - DNA, 21 bp, polyomavirus
ttttgcagtggaacctggcac

SEQ ID NO: 27 - DNA, 193 bp, polyomavirus
  misc_feature (1)..(193): contig 1378
attgaaggaggcctgttatattttattaccttaggcaagcatagagtttctgctgtaaaacatttttgtgttgcacagtgtacttttagtttcattcattgtaaagctgttattaaacctttagagctttatagagctttaggtaaacccccttttaagttgttggaagaaaacaagcctggtgtatccatgt

SEQ ID NO: 28 - DNA, 23 bp, polyomavirus
accttaggcaagcatagagtttc

SEQ ID NO: 29 - DNA, 23 bp, polyomavirus
atggatacaccaggcttgttttc

SEQ ID NO: 30 - DNA, 240 bp, polyomavirus
  misc_feature (1)..(240): contig 4636
cttcagtgccctgataaattctgacttcttccacttgccctgaagttccttccatgggttgtccctgaatttgaggcataagaccagagaacaagctattaaggagagagcttacaggataaggatttttaacaacccgtttccttagagttacattaaaatatctaggtaggcctctccagttttgggattctgaataattagtgtgaattccaacaatatcaacagcagacaaaaaca

SEQ ID NO: 31 - DNA, 21 bp, polyomavirus
tctgacttcttccacttgccc

SEQ ID NO: 32 - DNA, 21 bp, polyomavirus
cccaaaactggagaggcctac

SEQ ID NO: 33 - DNA, 290 bp, polyomavirus
  misc_feature (1)..(290): contig 6390
atctgaatgctgctctagactggggagaatcactgtttcatgctgttggcagggaaatatggagaaatattatgaggcaagctactcagcaaattggatatacttctagagctttggctgtaaggggtactaatgaatttcaacatatgcttgctcaaattgcagaaaatgcaaggtgggccctaactaatgggcctattcatatttattctagtgttgaagaatattatagaggtttgccttcagtaaatcctatacaactgagacaacagtatagaagtagaggagag

SEQ ID NO: 34 - DNA, 20 bp, polyomavirus
ctgaatgctgctctagactg

SEQ ID NO: 35 - DNA, 21 bp, polyomavirus
ctctcctctacttctatactg

SEQ ID NO: 36 - DNA, 273 bp, polyomavirus
  misc_feature (1)..(273): contig 6671
aattacttcttatttccagcaaggtaacttgcagcttttgaaataattcatttaatctttgcattttctcaggattaccacctttatcagggtggtaaattttggaaactgttttgtaggcctttttcataagagataaatttccccatgctgctcttgtaagttgcagcaaatccataagctcatttctctcctccaaagacagagtttgatccatggctctgcaaaaagtaaaataagtcttactacctgagaatcaagttaattaagttt

SEQ ID NO: 37 - DNA, 20 bp, polyomavirus
tccagcaaggtaacttgcag

SEQ ID NO: 38 - DNA, 20 bp, polyomavirus
actctgtctttggaggagag

SEQ ID NO: 39 - DNA, 24 bp, polyomavirus
aattacttcttatttccagcaagg

SEQ ID NO: 40 - DNA, 25 bp, polyomavirus
aaacttaattaacttgattctcagg

SEQ ID NO: 41 - DNA, 252 bp, polyomavirus
  misc_feature (1)..(252): contig 7646
aagtgtgtcacaggtcatgtcttcatttagcatgggtagcttaattacagccactgagtaagtagggagggtagtagcattagggttatcactagccttgctggaggcaacatttatatctgcactgtagccatacaattcatcagtagggttattgtttcccattcttggatttaggtaggcctcaatttgagttatagcatctgggcctgttctaacttctagaacttctacccctcctttgacaaggag

SEQ ID NO: 42 - DNA, 21 bp, polyomavirus
acagccactgagtaagtaggg

SEQ ID NO: 43 - DNA, 21 bp, polyomavirus
tccttgtcaaaggaggggtag

SEQ ID NO: 44 - DNA, 134 bp, polyomavirus
  misc_feature (1)..(134): Contig 5689
aacaaacatcttcaacctgggcagggagttaataatttggataatttaagagatcacttagatggtactattaaagttaatctggaaaaaaaacatgtgaataaaagaagccagatatttcctcctgtaattat

SEQ ID NO: 45 - DNA, 124 bp, polyomavirus
  misc_feature (1)..(124): Contig 1884
atgtggagagtggtaccctgcactgggatagttcctgaggatgagccatagatatatttacccccctgatgcagatttactagagaactaatccccatgacttcagtttttactgacacagcct

SEQ ID NO: 46 - DNA, 110 bp, polyomavirus
  misc_feature (1)..(110): Contig 4639
taatgttgtacatagtgtccagatctaggctctgaaccccctatctcccgcctcagcctaacttgttcctgatattcaaattgttccctagtaggggatgaatcttaaga

SEQ ID NO: 47 - DNA, 141 bp, polyomavirus
  misc_feature (1)..(141): Contig 4874
ctactactgaagcagcttggttaattgaaatagaagctgtagatgtagcaggcctaggagccttagaggccttaactttgactggactgtcagcagaagaattttctctacttagtgctctaccaactgccctgaacaatg

SEQ ID NO: 48 - DNA, 159 bp, polyomavirus
  misc_feature (1)..(159): Contig 2412
atgctatctatagtaataagactatgaattgctttttaatttatactacagttgaaaaaagtaagcagctttacagaactgttgaaaaatcaaaaattaaagtagattttaaggctattttcttgtataaagatgatggaattgaaggaggcctgttat

SEQ ID NO: 49 - DNA, 160 bp, polyomavirus
  misc_feature (1)..(160): Contig 6783
ctccagtgaagctcccatagtatcttgtattctcatttttagaggggtctgggctccaaacttctacagggtactttccatctttgtctagcaaagctttagcattaggatccaaggcctggtttactggtttcatatttttaatagttaccatatcagt

SEQ ID NO: 50 - DNA, 122 bp, polyomavirus
  misc_feature (1)..(122): Contig 5689 LPV
caaccaggtcaaggggtaaataatcttgataacctgagagatcatcttgatggaacaattaaagttaatttagaaaagaaacatgtaaacaaaaggagtcaaatttttcccccggttattat

SEQ ID NO: 51 - DNA, 122 bp, polyomavirus
  misc_feature (1)..(122): IPPyv contig 5689
caacctgggcagggagttaataatttggataatttaagagatcacttagatggtactattaaagttaatctggaaaaaaaacatgtgaataaaagaagccagatatttcctcctgtaattat

SEQ ID NO: 52 - DNA, 244 bp, polyomavirus
  misc_feature (1)..(244): LPV contig 649
cattttactagataattcttgggatgtttttcaaaatatcctacaattaataactaccagccaacccaaaaaaaggaatgtcctgataaagggaccaattaacagtggtaaaactactttggcttctgctttcatgcatttttttgatggcaaagctctaaatataaattgtcctgcagataaactgtcctttgaacttggctgtgctattgatcaattctgtgttttgttagatgatgtgaag

SEQ ID NO: 53 - DNA, 244 bp, polyomavirus
  misc_feature (1)..(244): IPPyV contig 649
cattttacttgacaactcatgggatgtttttcaaaaaattttgcagctggtaaccacaagccaaccaaaaaagaggaacatactctttaaaggccctataaatagtggcaaaaccaccttagcttctgctttcatgcacttttttgatggtaaagctttgaatattaactgcccagcagaaaaattatcttttgagttaggctgtgctattgatcaattttgtgttttgcttgatgatgtgaag

SEQ ID NO: 54 - DNA, 172 bp, polyomavirus
  misc_feature (1)..(172): LPV contig 650
tgtatagtatgtctgctaaaaaagcagcatgcaggtacaaaaaaaaatttaaaaaagccatgtttagtctggggagaatgttggtgctacaaatgttatttagtatggtttggctttcctgaggatttcacctcttttcgctactggacccttcttatggcaaatatggatt

SEQ ID NO: 55 - DNA, 172 bp, polyomavirus
  misc_feature (1)..(172): IPPyV contig 650
tgcattgtatgtttattaaagcagcagcacaaaagtacaaaaataaataaaaaaaagccttgcttagtatggggtgaatgcttttgctacaagtgctacttactttggtttggatttcctgaggattttacatcttttaactactggaccttattaatgagaaatatggatt

SEQ ID NO: 56 - DNA, 328 bp, polyomavirus
  misc_feature (1)..(328): LPV contig 816
tgttgcttggcatctacatagattttgcagtggaacctggcaaatgtggcaagtgtgaaaaaaagcagcacaaattccactataattatcacaaagcacatcatgccaatgcttgcctcttcttggagagtagagcccaaaaaaacatttgccaacaagcagttgaccaggtcctagcagctaaaaggttaaaattagtagaatgcagtagaattgaattattagaagagagatttttgcagctttttgatgaaatggatgacttcctgcatggtgagatagaaattctaagatggatggcgggtgtggcctggtacaccattttact

SEQ ID NO: 57 - DNA, 328 bp, polyomavirus
  misc_feature (1)..(328): IPPyV contig 816
tgttgcttggcatctacctagattttgcagtggaacctggcacttgctctaaatgtgaaaaaaagagtcataaattccattataattaccacagtaagcatcatgccaatgcttgccttttcttggagagtaaatcccaaaagaatatctgccaacaagcagttgaccaggtccttgcagctaagagattaaagcttgtagagtgtactagaatggagcttttagaagatagatttattcaactttttgatgaaatggaggactttttgcatggggaaattgaaattctaagatggatgtcaggtgtggcctggtacaccattttact

SEQ ID NO: 58 - DNA, 173 bp, polyomavirus
  misc_feature (1)..(173): LPV contig 1378
tttaattactttaggaaaacatagagtgtctgctgttaagcatttttgtgtatcccaatgtacttttagttttattcattgtaaagctgttgttaaacctctagagttatataagaccttaagtaaaccaccttttaagttgttggaagagaacaaaccgggtgtatccatgt

SEQ ID NO: 59 - DNA, 173 bp, polyomavirus
  misc_feature (1)..(173): IPPyV contig 1378
ttttattaccttaggcaagcatagagtttctgctgtaaaacatttttgtgttgcacagtgtacttttagtttcattcattgtaaagctgttattaaacctttagagctttatagagctttaggtaaacccccttttaagttgttggaagaaaacaagcctggtgtatccatgt

SEQ ID NO: 60 - DNA, 113 bp, polyomavirus
  misc_feature (1)..(113): LPV contig 1884
gtagtgccctgcacggggacacaccctgaggatgacccatagatgtactttcctccctggtgcaaattaactagtgaggaaattccaacaacttcagtctttacagacactgc

SEQ ID NO: 61 - DNA, 113 bp, polyomavirus
  misc_feature (1)..(113): IPPyV contig 1884
gtggtaccctgcactgggatagttcctgaggatgagccatagatatatttacccccctgatgcagatttactagagaactaatccccatgacttcagtttttactgacacagc

SEQ ID NO: 62 - DNA, 240 bp, polyomavirus
  misc_feature (1)..(240): LPV contig 4636
cttctgttccctcaaatattctgacttcttccacttgtcctgagaccccttccattggttgtccctgaatttggggcataagaccagagaagaagctatttagaagagagctgacaggataaggatttttaacaatccttttcctgagggtcacattgaaatatctgggaagccccctccaactttgggtttcagaatagttggtatgaactccagcaatatcagcacaagacagaaaca

SEQ ID NO: 63 - DNA, 240 bp, polyomavirus; identical to SEQ ID NO: 30
  misc_feature (1)..(240): IPPyV contig 4636

SEQ ID NO: 64 - DNA, 15 bp, polyomavirus
  misc_feature (1)..(15): LPV contig 4639
ttcttgatattcaaa

SEQ ID NO: 65 - DNA, 15 bp, polyomavirus
  misc_feature (1)..(15): IPPyV contig 4639
ttcctgatattcaaa

SEQ ID NO: 66 - DNA, 137 bp, polyomavirus
  misc_feature (1)..(137): LPV contig 4874
cattgttgagagctgttgggatagcacttaggagggaaaactgctctgttgtaagtccagtaagagacaaggcctctagagtactaagtccagcaagatccactgcttctatttcaatgagccaggctgcctcagta

SEQ ID NO: 67 - DNA, 137 bp, polyomavirus
  misc_feature (1)..(137): IPPyV contig 4874
cattgttcagggcagttggtagagcactaagtagagaaaattcttctgctgacagtccagtcaaagttaaggcctctaaggctcctaggcctgctacatctacagcttctatttcaattaaccaagctgcttcagta

SEQ ID NO: 68 - DNA, 288 bp, polyomavirus
  misc_feature (1)..(288): LPV contig 6390
ctctcctctgtttctatattcttgtcttagctggatgggatttctagcaggtagatacctgtaataatcttggacactggaataaatatggacaggcccactggtcaaagcccacctagcattttcagcaatttgagccaaggtgtgactcagttcattggtacttctgacagccacagcccttgtagcttgaccaatttgcaaagtggcctgtctcatcaaatgcctccatacttctctgcccacagcatgaaacaatgattcaccccagtcaagtacagcattcag

SEQ ID NO: 69 - DNA, 288 bp, polyomavirus
  misc_feature (1)..(288): IPPyV contig 6390
ctctcctctacttctatactgttgtctcagttgtataggatttactgaaggcaaacctctataatattcttcaacactagaataaatatgaataggcccattagttagggcccaccttgcattttctgcaatttgagcaagcatatgttgaaattcattagtaccccttacagccaaagctctagaagtatatccaatttgctgagtagcttgcctcataatatttctccatatttccctgccaacagcatgaaacagtgattctccccagtctagagcagcattcag

SEQ ID NO: 70 - DNA, 247 bp, polyomavirus
  misc_feature (1)..(247): LPV contig 6671
aggtaattaaatttattccattttattcacagccatggaccaaacgctgtctaaggaggagagaaatgagcttatggatttattgcaaataactagagctgcatggggaaatctttctatgatgaaaaaagcctataaaaatgtctccaagctctaccatcctgataaaggaggagattcagctaaaatgcagcggctcaatgaattatttcaaagggtccaggttaccttgatggagataaggagt

SEQ ID NO: 71 - DNA, 247 bp, polyomavirus
  misc_feature (1)..(247): IPPyV contig 6671
aggtagtaagacttattttactttttgcagagccatggatcaaactctgtctttggaggagagaaatgagcttatggatttgctgcaacttacaagagcagcatggggaaatttatctcttatgaaaaaggcctacaaaacagtttccaaaatttaccaccctgataaaggtggtaatcctgagaaaatgcaaagattaaatgaattatttcaaaagctgcaagttaccttgctggaaataagaagt

SEQ ID NO: 72 - DNA, 248 bp, polyomavirus
  misc_feature (1)..(248): LPV contig 7646
gtgtcacaggtcatgtcttcatttaggagggggagtttaataacagctactgaataacaaggaagggtgcctttgttgggggtgtcagaggccttactgaaagcagtatttatagaattactatatccatacaagtcctcagaaggaatattatttcccattctaggattaagataggcctcaatttgggtaatagcatcaggccctgttcttacttctagcacctctactcctccttttactaggag

SEQ ID NO: 73 - DNA, 248 bp, polyomavirus
  misc_feature (1)..(248): IPPyV contig 7646
gtgtcacaggtcatgtcttcatttagcatgggtagcttaattacagccactgagtaagtagggagggtagtagcattagggttatcactagccttgctggaggcaacatttatatctgcactgtagccatacaattcatcagtagggttattgtttcccattcttggatttaggtaggcctcaatttgagttatagcatctgggcctgttctaacttctagaacttctacccctcctttgacaaggag

SEQ ID NO: 74 - DNA, 140 bp, polyomavirus
  misc_feature (1)..(140): LPV contig 2412
atgcaatttatagtaataagactatgaatagttttttaatatatactactttggagaaagccaggcaactgtataaaactgtggaaaaatctaaaattgtagttgattttaaggctagtttttcttatcaggatgaggaa

SEQ ID NO: 75 - DNA, 141 bp, polyomavirus
  misc_feature (1)..(141): IPPyV contig 2412
atgctatctatagtaataagactatgaattgctttttaatttatactacagttgaaaaaagtaagcagctttacagaactgttgaaaaatcaaaaattaaagtagattttaaggctattttcttgtataaagatgatggaa

SEQ ID NO: 76 - DNA, 156 bp, polyomavirus
  misc_feature (1)..(156): LPV  contig  6783
cctgtaaaactcccataatatctagtattttcatttttagaggggtcagggcaccacacctccactgggtactttccatctttatccagcaaggctttggcctttggatctaggccttggtttcctggtttcatatttttaatagcaactacatca

SEQ ID NO: 77 - DNA, 156 bp, polyomavirus
  misc_feature (1)..(156): IPPyV contig 6783
ccagtgaagctcccatagtatcttgtattctcatttttagaggggtctgggctccaaacttctacagggtactttccatctttgtctagcaaagctttagcattaggatccaaggcctggtttactggtttcatatttttaatagttaccatatca

SEQ ID NO: 78 - protein, 368 aa, polyomavirus
  MISC_FEATURE (1)..(368): LPV VP1
MAPQRKRQDGACKKTCPIPAPVPRLLVKGGVEVLEVRTGPDAITQIEAYLNPRMGNNIPSEDLYGYSNSINTAFSKASDTPNKGTLPCYSVAVIKLPLLNEDMTCDTILMWEAVSVKTEVVGISSLVNLHQGGKYIYGSSSGCVPVQGTTYHMFAVGGEPLELQGLVASSTATYPDDVVAIKNMKPGNQGLDPKAKALLDKDGKYPVEVWCPDPSKNENTRYYGSFTGGATTPPVMQFTNSVTTVLLDENGVGPLCKGDKLFLSCADIAGVHTNYSETQSWRGLPRYFNVTLRKRIVKNPYPVSSLLNSFFSGLMPQIQGQPMEGVSGQVEEVRIFEGTEGLPGDPDLNRYVDKFCQNQTVLPVSNDM

SEQ ID NO: 79 - DNA, 1107 bp, polyomavirus
  misc_feature (1)..(1107): LPV VP1
tcacatatcatttgagacagggagaacagtctggttctgacaaaatttatcaacatatctattaaggtcagggtcccctgggagtccttctgttccctcaaatattctgacttcttccacttgtcctgagaccccttccattggttgtccctgaatttggggcataagaccagagaagaagctatttagaagagagctgacaggataaggatttttaacaatccttttcctgagggtcacattgaaatatctgggaagccccctccaactttgggtttcagaatagttggtatgaactccagcaatatcagcacaagacagaaacagtttgtcccctttacaaagaggcccaactccattttcatccagcagcacagttgtgacagaattagtgaactgcataactggtggggtggtggctccccctgtaaaactcccataatatctagtattttcatttttagaggggtcagggcaccacacctccactgggtactttccatctttatccagcaaggctttggcctttggatctaggccttggtttcctggtttcatatttttaatagcaactacatcatcaggataggtagctgtagagctagcaactaggccttggagttccaggggctctcctccaacagcaaacatgtgataggtagtgccctgcacggggacacaccctgaggatgacccatagatgtactttcctccctggtgcaaattaactagtgaggaaattccaacaacttcagtctttacagacactgcttcccacatcaaaatggtgtcacaggtcatgtcttcatttaggagggggagtttaataacagctactgaataacaaggaagggtgcctttgttgggggtgtcagaggccttactgaaagcagtatttatagaattactatatccatacaagtcctcagaaggaatattatttcccattctaggattaagataggcctcaatttgggtaatagcatcaggccctgttcttacttctagcacctctactcctccttttactaggagcctggggacgggagcgggaatagggcatgtttttttgcatgctccgtcttgcctttttctttgaggggccat

SEQ ID NO: 80 - protein, 423 aa, polyomavirus
  MISC_FEATURE (1)..(423): LPV  - VP1
MAPKRKASSTCKTPKRQCIPKPGCCPNVASVPKLLVKGGVEVLSVVTGEDSITQIELYLNPRMGVNSPDLPTTSNWYTYTYDLQPKGSSPDQPIKENLPAYSVARVSLPMLNEDITCDTLQMWEAISVKTEVVGISSLINVHYWDMKRVHDYGAGIPVSGVNYHMFAIGGEPLDLQGLVLDYQTEYPKTTNGGPITIETVLGRKMTPKNQGLDPQAKAKLDKDGNYPIEVWCPDPSKNENSRYYGSIQTGSQTPTVLQFSNTLTTVLLDENGVGPLCKGDGLFISCAHIVGFLFKTSGKMALHGLPRYFNVTLRKIWVKNPYPVVNLINSLFSNLMPKVSGQPMEGKDNQVEEVRIYEGSEQLPGNPDIVRFLDKFGQEKTVYPKPSVAPAAVTFQSNQQDKGKAPLKGPQKASQKESQTQQL

SEQ ID NO: 81 - protein, 697 aa, polyomavirus
MDQTLSKEERNELMDLLQITRAAWGNLSMMKKAYKNVSKLYHPDKGGDSAKMQRLNELFQRVQVTLMEIRSQCGSSSSQGYFSEDFYFGPTTFQYSPMDRDAVREDLPNPGEGSWGKWWREFVNRQCCDDLFCSETMSSSSDEDTPPAAQPPPPPAPSPEEEDEIEFVEETPSSCDGSSSQSSYTCTPPKRKKTEEKKPDDFPVCLYSFLSHAIYSNKTMNSFLIYTTLEKARQLYKTVEKSKIVVDFKASFSYQDEEGEGCLLFLITLGKHRVSAVKHFCVSQCTFSFIHCKAVVKPLELYKTLSKPPFKLLEENKPGVSMFEFQEEKEQSVNWQEICNFANEANISDVLLLLGIYIDFAVEPGKCGKCEKKQHKFHYNYHKAHHANACLFLESRAQKNICQQAVDQVLAAKRLKLVECSRIELLEERFLQLFDEMDDFLHGEIEILRWMAGVAWYTILLDNSWDVFQNILQLITTSQPKKRNVLIKGPINSGKTTLASAFMHFFDGKALNINCPADKLSFELGCAIDQFCVLLDDVKGQITLNKHLQPGQGVNNLDNLRDHLDGTIKVNLEKKHVNKRSQIFPPVIMTMNEYLLPPTIGVRFALHLHLKPKAYLKQSLEKSDLVAKRILNSGYTILLLLLWYNPVDSFTPKVQEKVVQWKETLEKYVSITQFGNIQQNIIDGKDPLHGIVIEEQM

SEQ ID NO: 82 - DNA, 5028 bp, polyomavirus
ccccttgcctccttctcttgttgcaacaaagagagaggctttggaggcttttccaaaactcattaaggtaagaagttctccacctatttgaaacttaattaacttgattctcaggtagtaagacttattttactttttgcagagccatggatcaaactctgtctttggaggagagaaatgagcttatggatttgctgcaacttacaagagcagcatggggaaatttatctcttatgaaaaaggcctacaaaacagtttccaaaatttaccaccctgataaaggtggtaatcctgagaaaatgcaaagattaaatgaattatttcaaaagctgcaagttaccttgctggaaataagaagtaattgtggatcctcctcttctcaggtagcttggtacttttgggatgagaattttagaacactaggtgcttttctaggagaaaaatttaatcaaagaattattggtggatatcctgattgcattacctataataagcccagttgttgctgcattgtatgtttattaaagcagcagcacaaaagtacaaaaataaataaaaaaaagccttgcttagtatggggtgaatgcttttgctacaagtgctacttactttggtttggatttcctgaggattttacatcttttaactactggaccttattaatgagaaatatggatttaagtttgctgcgcctctggactgagcttggattctaatgtaagtatttttttttttagggatactacagtgattctccatactttactgaaactccattttcctactgtgaaagaaaaaatgaagatccagaaggggggtcatggggtaaatggtggagggagtttgttaacaaagaatatgatgatttattttgttcagagactatatcatctagtgatgatgaaaataatcctggaccttcagcccctccaccctcttctgcttctgcctctgaagaccctgatcctgaggaagaagctggatcatcccagagttcatttacctgcacgccccccaaaagaaagaagcctgaacctaatactccagaggactttcctatgtgtttgtattcttttttaagtcatgctatctatagtaataagactatgaattgctttttaatttatactacagttgaaaaaagtaagcagctttacagaactgttgaaaaatcaaaaattaaagtagattttaaggctattttcttgtataaagatgatggaattgaaggaggcctgttatattttattaccttaggcaagcatagagtttctgctgtaaaacatttttgtgttgcacagtgtacttttagtttcattcattgtaaagctgttattaaacctttagagctttatagagctttaggtaaacccccttttaagttgttggaagaaaacaagcctggtgtatccatgtttgactttcaggaggaaaaagaacaggctgtaaattggcaggagatttgtaattatgcggttgaagcaaaaattactgatgtattactgttgcttggcatctacctagattttgcagtggaacctggcacttgctctaaatgtgaaaaaaagagtcataaattccattataattaccacagtaagcatcatgccaatgcttgccttttcttggagagtaaatcccaaaagaatatctgccaacaagcagttgaccaggtccttgcagctaagagattaaagcttgtagagtgtactagaatggagcttttagaagatagatttattcaactttttgatgaaatggaggactttttgcatggggaaattgaaattctaagatggatgtcaggtgtggcctggtacaccattttacttgacaactcatgggatgtttttcaaaaaattttgcagctggtaaccacaagccaaccaaaaaagaggaacatactctttaaaggccctataaatagtggcaaaaccaccttagcttctgctttcatgcacttttttgatggtaaagctttgaatattaactgcccagcagaaaaattatcttttgagttaggctgtgctattgatcaattttgtgttttgcttgatgatgtgaagggccagataactttaaacaaacatcttcaacctgggcagggagttaataatttggataatttaagagatcacttagatggtactattaaagttaatctggaaaaaaaacatgtgaataaaagaagccagatatttcctcctgtaattatgacaatgaatgagtatttactcccacccacagtgggagttagatttgctttgcacatccattttcattgtaagacctacctgaagcagagtttagaaaaaagtgacttaattgaaaaaagaattcttaactctggttacacaatcttgctgttacttttatggtacaaccctgttgattcttttactccaaaagtccaggaatatgttgtaaaatggaaagagattttagagagacatgtatctattactcagtttggaaatatccagcaaaacattcttgatggtaaagatcccctgcatggaattgttattgaagaacaagcataacaatttgtacttttcttcttcagatttattcatggttttgttaatacaataaagctttacaatgcaaccagcctcattgatcatttgagcgaggggggacagtttgattctgacaaaatttatcaacatatctatcaagatcagggtccccgggtaggccttcagtgccctgataaattctgacttcttccacttgccctgaagttccttccatgggttgtccctgaatttgaggcataagaccagagaacaagctattaaggagagagcttacaggataaggatttttaacaacccgtttccttagagttacattaaaatatctaggtaggcctctccagttttgggattctgaataattagtgtgaattccaacaatatcaacagcagacaaaaacagcttgtcccctttacaaagaggccccactccattttcatccaatagcaccgtggttacagagttagtaaactgcataacagggggggttgtggctcctccagtgaagctcccatagtatcttgtattctcatttttagaggggtctgggctccaaacttctacagggtactttccatctttgtctagcaaagctttagcattaggatccaaggcctggtttactggtttcatatttttaatagttaccatatcagtaggataggtagtagtagagctagcaactaggccttgaagttcaagaggctctccacccactgaaaacatgtggagagtggtaccctgcactgggatagttcctgaggatgagccatagatatatttacccccctgatgcagatttactagagaactaatccccatgacttcagtttttactgacacagcctcccacattagaagtgtgtcacaggtcatgtcttcatttagcatgggtagcttaattacagccactgagtaagtagggagggtagtagcattagggttatcactagccttgctggaggcaacatttatatctgcactgtagccatacaattcatcagtagggttattgtttcccattcttggatttaggtaggcctcaatttgagttatagcatctgggcctgttctaacttctagaacttctacccctcctttgacaaggagtttgggaacgggagcgggtgttggacaggttttttttacaggacatgctccgcactcttgcctttttctttggggggccatattcttctttttccaatttactcagctctttctcccaggttggagttatatcaccatacaaacctagaattagaggaagcatccaatcttgagatactctttgatttgcccctcctggagcagcataatgttgtacatagtgtccagatctaggctctgaaccccctatctcccgcctcagcctaacttgttcctgatattcaaattgttccctagttgggggtagctctcctctacttctatactgttgtctcagttgtataggatttactgaaggcaaacctctataatattcttcaacactagaataaatatgaataggcccattagttagggcccaccttgcattttctgcaatttgagcaagcatatgttgaaattcattagtaccccttacagccaaagctctagaagtatatccaatttgctgagtagcttgcctcataatatttctccatatttccctgccaacagcatgaaacagtgattctccccagtctagagcagcattcagatagtaactaaaagaagacaagcctggaaacagatagtccacttgaggaaaccaaggagtgagagccatactaacaagtggtacttctttggaatatccaaaagttgttactcctgcagcaaccacagcactggcacctgtaacagtttgaaaaaagattcctattcctatggcattgttcagggcagttggtagagcactaagtagagaaaattcttctgctgacagtccagtcaaagttaaggcctctaaggctcctaggcctgctacatctacagcttctatttcaattaaccaagctgcttcagtagtagcagcagctatagcttccccagacaatattgcatcaacagtaaatccagtacttaaacttaattgttcagccagttcaaaaatatccaacaatatagttaaaaccccccccatatcttacccaagttcaaaattggcgcgtttagtttaggcgggcttcttctctgttgaccttagatggcaaaatgatttcacttcccccttagctactggtatctagaggggttgctaagcgccacctagcaacggaaaccgcaatgcacttgtttgtcacacaccggaatgtcaccaaaaagtccccaggcagtcaagtggagcaggtagataccacagataccacagtttcctctagagttgattctgcaaaaataacctgtcatttagttcctcaatttgcaaagttatctccctgtcactcaaaatcactatggcaacacaggtttttttttgatagtataagaggccagggg

SEQ ID NO: 83 - DNA, 2043 bp, polyomavirus
  CDS (1)..(2043)
  misc_feature (1)..(2043): Large T DNA
atggatcaaactctgtctttggaggagagaaatgagcttatggatttgctgcaacttacaagagcagcatggggaaatttatctcttatgaaaaaggcctacaaaacagtttccaaaatttaccaccctgataaaggtggtaatcctgagaaaatgcaaagattaaatgaattatttcaaaagctgcaagttaccttgctggaaataagaagtaattgtggatcctcctcttctcagggatactacagtgattctccatactttactgaaactccattttcctactgtgaaagaaaaaatgaagatccagaaggggggtcatggggtaaatggtggagggagtttgttaacaaagaatatgatgatttattttgttcagagactatatcatctagtgatgatgaaaataatcctggaccttcagcccctccaccctcttctgcttctgcctctgaagaccctgatcctgaggaagaagctggatcatcccagagttcatttacctgcacgccccccaaaagaaagaagcctgaacctaatactccagaggactttcctatgtgtttgtattcttttttaagtcatgctatctatagtaataagactatgaattgctttttaatttatactacagttgaaaaaagtaagcagctttacagaactgttgaaaaatcaaaaattaaagtagattttaaggctattttcttgtataaagatgatggaattgaaggaggcctgttatattttattaccttaggcaagcatagagtttctgctgtaaaacatttttgtgttgcacagtgtacttttagtttcattcattgtaaagctgttattaaacctttagagctttatagagctttaggtaaacccccttttaagttgttggaagaaaacaagcctggtgtatccatgtttgactttcaggaggaaaaagaacaggctgtaaattggcaggagatttgtaattatgcggttgaagcaaaaattactgatgtattactgttgcttggcatctacctagattttgcagtggaacctggcacttgctctaaatgtgaaaaaaagagtcataaattccattataattaccacagtaagcatcatgccaatgcttgccttttcttggagagtaaatcccaaaagaatatctgccaacaagcagttgaccaggtccttgcagctaagagattaaagcttgtagagtgtactagaatggagcttttagaagatagatttattcaactttttgatgaaatggaggactttttgcatggggaaattgaaattctaagatggatgtcaggtgtggcctggtacaccattttacttgacaactcatgggatgtttttcaaaaaattttgcagctggtaaccacaagccaaccaaaaaagaggaacatactctttaaaggccctataaatagtggcaaaaccaccttagcttctgctttcatgcacttttttgatggtaaagctttgaatattaactgcccagcagaaaaattatcttttgagttaggctgtgctattgatcaattttgtgttttgcttgatgatgtgaagggccagataactttaaacaaacatcttcaacctgggcagggagttaataatttggataatttaagagatcacttagatggtactattaaagttaatctggaaaaaaaacatgtgaataaaagaagccagatatttcctcctgtaattatgacaatgaatgagtatttactcccacccacagtgggagttagatttgctttgcacatccattttcattgtaagacctacctgaagcagagtttagaaaaaagtgacttaattgaaaaaagaattcttaactctggttacacaatcttgctgttacttttatggtacaaccctgttgattcttttactccaaaagtccaggaatatgttgtaaaatggaaagagattttagagagacatgtatctattactcagtttggaaatatccagcaaaacattcttgatggtaaagatcccctgcatggaattgttattgaagaacaagcataa

SEQ ID NO: 84 - protein, 680 aa, polyomavirus
  MISC_FEATURE (1)..(680): Large T antigen
MDQTLSLEERNELMDLLQLTRAAWGNLSLMKKAYKTVSKIYHPDKGGNPEKMQRLNELFQKLQVTLLEIRSNCGSSSSQGYYSDSPYFTETPFSYCERKNEDPEGGSWGKWWREFVNKEYDDLFCSETISSSDDENNPGPSAPPPSSASASEDPDPEEEAGSSQSSFTCTPPKRKKPEPNTPEDFPMCLYSFLSHAIYSNKTMNCFLIYTTVEKSKQLYRTVEKSKIKVDFKAIFLYKDDGIEGGLLYFITLGKHRVSAVKHFCVAQCTFSFIHCKAVIKPLELYRALGKPPFKLLEENKPGVSMFDFQEEKEQAVNWQEICNYAVEAKITDVLLLLGIYLDFAVEPGTCSKCEKKSHKFHYNYHSKHHANACLFLESKSQKNICQQAVDQVLAAKRLKLVECTRMELLEDRFIQLFDEMEDFLHGEIEILRWMSGVAWYTILLDNSWDVFQKILQLVTTSQPKKRNILFKGPINSGKTTLASAFMHFFDGKALNINCPAEKLSFELGCAIDQFCVLLDDVKGQITLNKHLQPGQGVNNLDNLRDHLDGTIKVNLEKKHVNKRSQIFPPVIMTMNEYLLPPTVGVRFALHIHFHCKTYLKQSLEKSDLIEKRILNSGYTILLLLLWYNPVDSFTPKVQEYVVKWKEILERHVSITQFGNIQQNILDGKDPLHGIVIEEQA

SEQ ID NO: 85 - DNA, 21 bp, Artificial
  primer VP1-354F
accatatcagtaggataggta

SEQ ID NO: 86 - DNA, 21 bp, Artificial
  primer VP1-354R
tgaattgtatggctacagtgc

SEQ ID NO: 87 - DNA, 20 bp, Artificial
  primer VP1-198F
cactgggatagttcctgagg

SEQ ID NO: 88 - DNA, 21 bp, Artificial
  primer VP1-198R
cctaatgctactaccctccct

(19) United States
(12) Patent Application Publication
(10) Pub. No.: US 2014/0024017 A1
(43) Pub. Date: Jan. 23, 2014

(54) IDENTIFICATION OF A NOVEL HUMAN POLYOMAVIRUS (IPPyV) AND APPLICATIONS

(71) Applicants: Marc Eloit, Paris (FR); Justine Cheval, Paris (FR); Virginie Sauvage, Reims (FR)

(72) Inventors: Marc Eloit, Paris (FR); Justine Cheval, Paris (FR); Virginie Sauvage, Reims (FR)

(21) Appl. No.: 13/977,043

(22) PCT Filed: Dec. 27, 2011
(86) PCT No.: PCT/EP2011/074112

§ 371 Date: Sep. 19, 2013

Related U.S. Application Data
(60) Provisional application No. 61/490,435, filed on May 26, 2011.

(30) Foreign Application Priority Data
Dec. 30, 2010 (EP) 10290686.4

(51) Int. Cl. C12Q 1/70; G01N 33/68

(52) U.S. Cl. CPC C12Q 1/701; G01N 33/68
USPC 435/5; 536/23.72; 435/320.1; 530/350; 530/389.4

(57) ABSTRACT

The invention relates to the identification of a novel human polyomavirus species (designated IPPyV) and applications derived from the identified features and properties of this virus. The IPPy virus species of the invention qualifies as a human virus, in view of the fact that it is capable of infecting a human host. Having identified a novel human polyomavirus species, IPPyV, the inventors have been able to propose means for the detection of exposure or infection by such a virus, especially detection in a biological sample previously obtained from a human host. The invention also concerns means suitable for obtaining an immune response in a host with a view to prevent the onset or the development of an infection with an IPPyV.

The invention relates to the identification of a novel human polyomavirus (designated IPPyV) and applications derived from the identified features and properties of this virus.

The invention is thus directed to a new polyomavirus species (IPPyV species), which has been identified in a biological sample of a human patient suffering from a Merkel cell carcinoma and relates to the constitutive components of said virus, including its genome and proteins and also relates to products expressed in cells as a result of exposure or infection with such a virus.

The IPPy virus species of the invention qualifies as a human polyomavirus virus, in view of the fact that it is capable of infecting a human host.

Having identified a novel human polyomavirus species, IPPyV, the inventors have been able to propose means for the detection of exposure or infection by such a virus, especially detection in a biological sample previously obtained from a human host. The invention also concerns means suitable for obtaining an immune response in a host with a view to prevent the onset or the development of an infection with IPPyV.

Polyomaviridae is a family of non-enveloped DNA viruses with a circular genome. Virions have VP1 capsid protein subunits arranged in pentavalent capsomeres. The genome consists of a single molecule of around 5 kb in size. The genome may persist in infected cells in an integrated form. It encodes three capsid proteins (VP1, VP2 and VP3) and a large and a small T antigen. Transcription of the genome is divided into early and late stages: each transcription step occurring on opposite DNA strands DNA replication starts at a fixed point on the genome, which remains under circular configuration during the process. Replication proceeds bidirectionnaly from a unique origin of replication and uses the action of host DNA polymerases. Replication and assembly of the virions is achieved in the nucleus and the virions are released mainly via cell destruction. Natural hosts for the viruses of the Polyomaviridae family are primates including humans and monkeys, rodents, cattle, rabbits and birds. Currently, the identified human members of this family are:
The JC virus (JCV) found in a patient (initials JC), with Hodgkin's lymphoma who suffered from progressive multifocal leukoencephalopathy (PML),
The BK virus (BKV) isolated from urine of a kidney transplant patient (initials)) BK,
The KI polyoma virus (KIPyV) identified from respiratory secretions of patients with respiratory infections at the Karolinska Institute (KI),
The WU polyomavirus (WUPyV) isolated in the same conditions et the Washington University (WU),
Merkel cell polyoma virus (MCPyV) associated with Merkel cell carcinoma,
The Trichodysplasia spinulosa-associated polyomavirus (TSPyV).

Among these previously described polyomaviruses, four viruses are known to cause diseases in humans: i) the BK virus causes an interstitial nephritis, known as BK virus nephropathy (BKVN) or polyoma virus associated nephropathy (PVAN) ii) the JC virus causes Progressive Multifocal Leucoencephalopathy PML in immunosuppressed patients iii) The MCPyV causes the Merkel cell carcinoma, a rare but very aggressive skin tumor iv) TSPyV causes Trichodysplasia spinulosa, a rare skin disease exclusively seen in immunocompromized patients (1).

Among previously described polyomaviruses, the Lymphotropic polyomavirus (LPV) has also been described in African Green Monkeys.

Having hypothesized the possibility of the occurrence of a different human member in the family of polyomaviruses, the inventors have determined conditions to achieve identification of such a virus, including selection of the target tissue sample for the presence of the virus or its characteristics features or product, methods to prepare appropriate biological material for the isolation of said virus and its product, sequencing strategies in order to obtain the viral genome and identification of biological properties of the obtained products.

Accordingly, the inventors have been able to identify the presence of the novel IPPyV virus in a biological sample, obtained from a patient known to be infected by another member of the polyomavirus family, i.e., a virus of the MCPy virus species.

In addition and as shown in the examples which will follow, the inventors have determined that such a previously unrecognized polyomavirus could be associated with a disease effect, in particular with the occurrence or the development of a carcinoma in a human host. In view of the association of the presence of the novel virus, possibly in conjunction with presence of another polyomavirus of the MCPy virus species, with a disease condition, especially with carcinoma or other cancers, there is obviously a need to specifically identify said virus and to provide means suitable for detection of exposure or infection of a human host by such a virus.

There is also obviously a need for devising means suitable for clearance of the virus in a human host or for treatment of the disease condition associated with such an infection. The present invention provides such means or provides products and tools for their design.

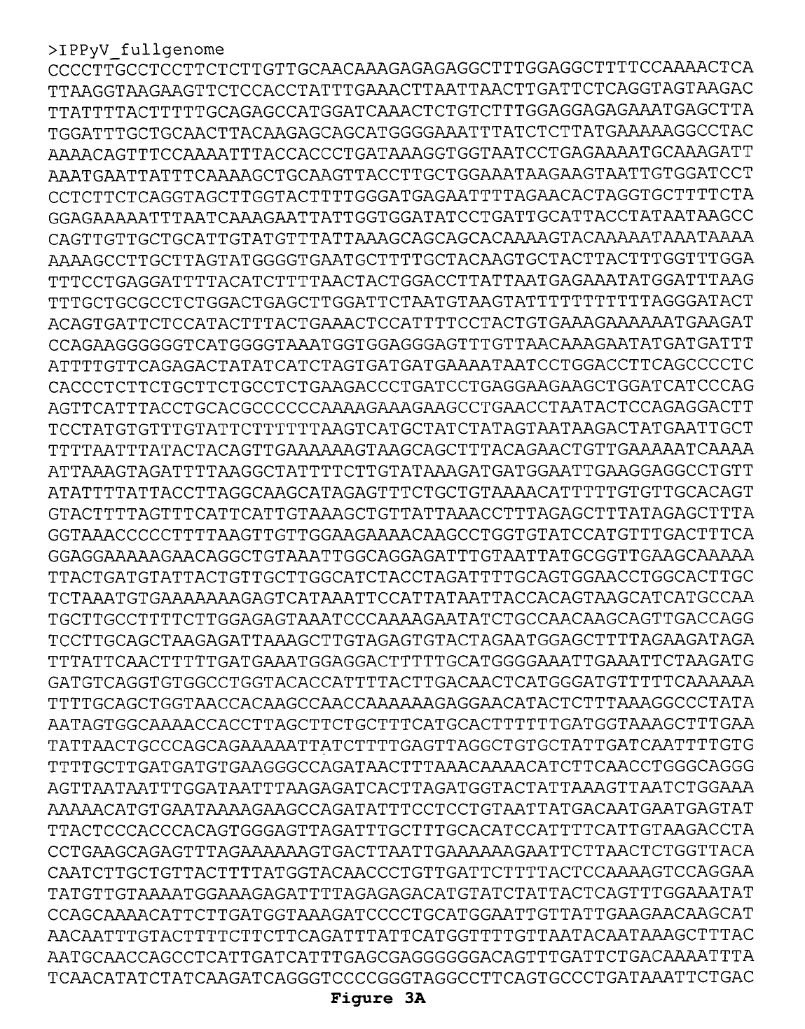

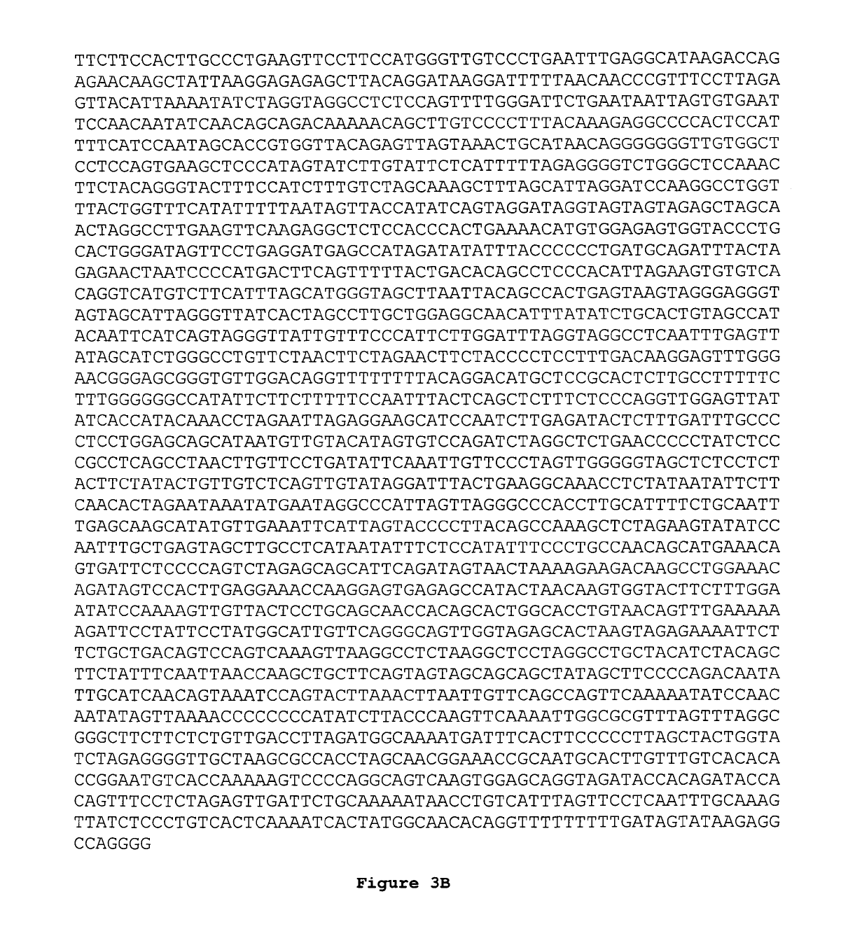

The invention thus relates to a polynucleotide which is selected from the group consisting of:
(a) a polynucleotide which comprises or consists in a nucleic acid with sequence disclosed as SEQ ID N°1 (FIG. 3) or a nucleic acid having an inverse complementary sequence or;
(b) a polynucleotide which comprises or consists in a nucleic acid having sequence disclosed as either SEQ ID N°2 (VP1) or SEQ ID N°4 (VP2) or SEQ ID N°6 (VP3) or SEQ ID N°8 (Large T) or SEQ ID N°10 (Small T), or,
(c) a polynucleotide which hybridizes with a nucleic acid of either of (a) or (b) in stringent conditions or,
(d) a polynucleotide variant which has the same size as the polynucleotide having the nucleic acid sequence of SEQ ID N° 1 and which has an identity of 75% or more especially at least one of the following thresholds: 75.8%, 80%, 85%, 90%, 95%, 98%, 99%, over its whole nucleic acid sequence when aligned with SEQ ID N° 1 or which has a smaller size than the polynucleotide having the nucleic acid sequence of SEQ ID N° 1 and which has an identity of 75% or more especially at least one of the following thresholds: 75.8%, 80%, 85%, 85%, 90%, 95%, 98%, 99%, over its nucleic acid sequence when aligned with the corresponding sequence in SEQ ID N° 1 or,
(e) a polynucleotide being a variant of one of the polynucleotides having the nucleic acid sequence of reference SEQ ID N°2 (VP1), SEQ ID N°4 (VP2), SEQ ID N°6 (VP3), SEQ ID N°8 (Large T), SEQ ID N°10 (Small T), which has the same size as the sequence of reference and which has an identity of respectively 44.9%, 77.5%, 75.0%, 60.2%, 78.6%, or more over its whole nucleic acid sequence when aligned with respectively one of the sequences of reference SEQ ID N°2, SEQ ID N°4, SEQ ID N°6, SEQ ID N°8, SEQ ID N°10, or which has a smaller size than the sequence of reference and which has an identity of at least one of the following thresholds: 50%, 60%, 70% 75%, 80%, 85%, 90%, 95%, 98%, 99% with the aligned sequence in the sequence of reference.

The sequence disclosed as SEQ ID N° 1 is also illustrated in FIG. 3 where the virus genome is also schematically represented. Its inverse complementary sequence (also referred to as complementary sequence) has opposite 5′ to 3′ orientation and can be deducted from SEQ ID N°1. Both strands of the genome comprise Open Reading Frames.

The expression “polynucleotide” defines any nucleic acid molecule and especially DNA or RNA molecules, either single-stranded or double-stranded molecules. In particular, a polynucleotide may be a genomic DNA molecule, a complementary DNA molecule (cDNA) obtained with reverse transcriptase enzyme and DNA polymerase or may be an RNA molecule in particular a messenger RNA (mRNA).

The polynucleotide may be isolated or purified from virus particles or from cells infected with virus particles or from cells infected with the virus. They may be cloned or obtained by amplification. They may be produced synthetically by any method known from the skilled person.

The polynucleotide of the invention encompasses variant polynucleotides defined with respect to the specific nucleic acid molecules having SED ID N° 1, SEQ ID N°2 (VP1), SEQ ID N°4 (VP2), SEQ ID N°6 (VP3), SEQ ID N°8 (Large T), SEQ ID N°10 (Small T) or their fragments, considered as reference sequences for the definition of said variants in relation to identity of the nucleic acid sequences.

These variants are molecules which have a modified size, especially a smaller size, and/or nucleotide content, with respect to the chosen sequence of reference.

According to a particular embodiment of the invention, the variant polynucleotides are defined with respect to their identity (determined as a percentage of identity) with the sequence of reference.

The variant polynucleotides may also in another embodiment be defined with respect to their hybridization properties with the sequence of reference.

The sequence of reference is either one of the herein listed nucleic acid sequences or a fragment thereof which is conceptually obtained by alignment (corresponding sequence) with the sequence of the variant or is a region in the corresponding sequence.

When defined with respect to sequence identity, the identity of the variant is measured as a percentage of identity, when the compared sequences are aligned for optimal comparison. The compared sequences may have the same length or may have different lengths. In this latter case, the identity is determined with respect to the number of nucleotides of the shorter sequence or with respect to a sequence forming a “comparison window” in said sequence. When the polynucleotide is a fragment of one of said sequences of reference, the “comparison window” is the sequence of the fragment or is a smaller region in it which is aligned for comparison. The skilled person would be able to rely on available algorithms for the comparison and especially can use mapping programs such as BLAST. Suitable algorithms for alignment and comparison are disclosed hereafter.

As stated above, the comparison of sequences is performed when considering the whole size of the available sequences or may be performed on the basis of a “comparison window”, corresponding to a conceptual restricted region or a plurality of such regions, in the whole sequences. This comparison window may be selected with respect to a particular structure of interest and/or with respect to a function or application of the compared sequences.

In a particular embodiment, the variant polynucleotide has the functional properties of the sequence of reference. In a particular embodiment, the variant polynucleotide has the property to hybridize with a complementary strand of the sequence of reference, or the property to encode a polypeptide having substantially the antigenic or the immunogenic properties of the polypeptide encoded by the sequence of reference.

In another embodiment the variant polynucleotide has altered properties or additional properties, including ability to be used in various processes in vitro, or to devise new products. In a particular embodiment, the variant polynucleotide is the genome, an ORF, or a fragment thereof as defined herein, of a IPPyV virus strain, especially an isolate obtained from a human patient or of a virus obtained from cultured cells or of a tissue sample or body fluid sample obtained from a human patient, possibly after amplification.

“Stringent conditions” are conditions that allow specific hybridization and accordingly only enable the formation of a hybrid between two aligned strands to form a double-stranded molecule when both strands of said molecule are sufficiently complementary to enable stable matching of the nucleotides facing each other when the sequences of said strands are aligned. The skilled person will be able to determine conditions that would be considered stringent and such conditions are illustrated as follows: overnight incubation at 42° C. in solution comprising: 50% formamide, 5×SSC (150 mM NaCl, 15 mM trisodium citrate), 50 mM sodium phosphate (pH 7.6), 5× Denhardt's solution, 10% dextran sulphate, and 20 μg/ml denatured sheared salmon sperm DNA followed by washing the filters in 0.1×SSC at about 65° C.

When a polynucleotide of the invention is defined with respect to the identity with a given nucleic acid sequence (sequence of reference) or with respect to the hybridization capacity of its nucleic acid sequence with the complementary sequence of a sequence of reference the nucleic acids defined herein, including SEQ ID N°1, SEQ ID N°2 (VP1), SEQ ID N°4 (VP2), SEQ ID N°6 (VP3), SEQ ID N°8 (Large T), SEQ ID N°10 (Small T) and their fragments may be the nucleic acids providing the sequence of reference.

The invention relates especially to a polynucleotide which is a nucleic acid having the sequence disclosed as SEQ ID N° 82 or a nucleic acid having the sequence disclosed as SEQ ID N° 83 (DNA coding large T antigen).

In a particular embodiment, the percentage of identity of a given sequence (compared sequence) with the sequence of reference SEQ ID N°1 may be at least one of the following: 75%, 75.8%, 80%, 85%, 90%, 95%, 98%, 99%,

The percentage of identity of a given sequence (compared sequence) with the sequence of reference SEQ ID N°2 (VP1) may be at least 44.9% and in particular at least 50%, at least 75%, at least 80%, at least 85%, at least 90%, at least 95%, at least 99%.

The percentage of identity of a given sequence (compared sequence) with the sequence of reference SEQ ID N°4 (VP2) may be at least 77.5%, at least 80%, at least 85%, at least 90%, at least 95%, at least 99%;

The percentage of identity of a given sequence (compared sequence) with the sequence of reference SEQ ID N°6 (VP3) may be at least 75.5%, at least 75%, at least 80%, at least 85%, at least 90%, at least 95%, at least 99%;

The percentage of identity of a given sequence (compared sequence) with the sequence of reference SEQ ID N°8 (Large T) may be at least 60.2%, at least 75%, at least 80%, at least 85%, at least 90%, at least 95%, at least 99%;

The percentage of identity of a given sequence (compared sequence) with the sequence of reference SEQ ID N°10 (Small T) may be at least 78.6%, at least 75%, at least 80%, at least 85%, at least 90%, at least 95%, at least 99%.

The “percentage of sequence identity” is calculated by comparing two optimally aligned sequences over the window of comparison, determining the number of positions at which the identical nucleic acid base (e.g. A, T, C, G, U, or I) occurs in both sequences to yield the number of matched positions, dividing the number of matched positions to by the total number of positions in the window of comparison (i.e. the window size) and multiplying the result by 100 to yield the percentage of sequence identity. The same process can be applied to polypeptide sequences. The percentage of sequence identity of two nucleic acid sequences or two amino acid sequences can be calculated using the Needleman-Wunsch global alignment algorithm to find the optimum alignment (including gaps) when considering their entire length parameter or the Smith-Waterman algorithm to calculate the local alignments (for example using the following server http://www.ebi.ac.uk/Tools/emboss/align/index.html).

In a particular embodiment, the comparison is made with respect to each Open Reading Frame determined in the sequence of FIG. 3 or in its complementary sequence and therefore is performed on the basis of each coding sequence of a polypeptide of the IPPyV.

In a particular embodiment of the invention, the polynucleotide is the double-stranded DNA of the genome of a human polyomavirus of the invention; i.e., of a IPPyV such as the one of FIG. 3, or is a single strand of said double-stranded genome or is a variant strain genome or a single strain from such a variant genome, or a fragment thereof, said variant strain genome or fragment being possibly derived by mutation and/or recombination from the reference genome illustrated in FIG. 3, including derived from a variant isolate obtained from a patient or a cellular form of the virus or an amplification variant, such as a PCR amplicon.

In a particular embodiment, the polynucleoptide consisting in or comprising the variant of the genome or genomic strands of the IPPyV is different as a result of the degeneracy of the genetic code and the obtained variant genome nevertheless encodes the ORF and especially the viral polypeptides as defined herein. In another embodiment of the invention, the variant genome encodes polypeptides structurally and/or functionally different from the polypeptides encoded by the ORF having sequences of SEQ ID N°2, SEQ ID N°4, SEQ ID N°6, SEQ ID N°8, SEQ ID N°10, SEQ ID N°83.

Another variant is a defective viral genome or single strand of a defective genome containing deletion(s), duplication(s) or rearrangement(s) of nucleotide(s) to the extent that said defective viral genome or single strand of the genome has an identity of 75% or more, especially of 75.8% or more, 80% or more, 85% or more, 90% or more, 95% or more, 98%, 99% in particular 99.5% and especially of 99.9% over its whole nucleic acid sequence when aligned with the sequence of SEQ ID N°1 or with its complementary strand.

Accordingly, unless otherwise specified the term “polynucleotide” encompasses variant polynucleotides as defined herein and encompasses fragments of the nucleic acids defined herein, including of the variants defined herein.

Another variant of the polynucleotide of the IPPyV genome or of one strand of said genome represented on FIG. 3, is a polynucleotide which is codon optimized for expression in specific cells. In particular, the polynucleotide may be codon optimized for expression in mammalian cells, especially in human cells.

As for other polyomaviruses identified in the prior art, the polyomavirus of the present invention may have a sequence that varies with respect to the sequence of SEQ ID N°1 or to its complementary strand, depending on the source of nucleic acid used (virus particles, infected cells, amplification product) to identify or prepare said polynucleotide.

The invention thus also concerns a set of polynucleotides, which comprises nucleic acids having polymorphisms, especially polymorphisms reflecting the origin of the polynucleotides such as virus particles, integrated forms found in the nucleus of the host cell.

Another embodiment of the invention relates to a polynucleotide which is a fragment of a polynucleotide defined herein with respect to the reference sequences SEQ ID N°1, SEQ ID N°2, SEQ ID N°4, SEQ ID N°6, SEQ ID N°8, SEQ ID N°10, SEQ ID N°82 or SEQ ID N°83 or with respect to their complementary sequences, including fragments of polynucleotide variants.

The size of the fragment may depend upon the intended use of said fragment, i.e., may be defined with respect to functional properties. Said size may be in the range of “n” to up to about 5000 nucleotides or less especially up to 4500, 4000, 3500, 3000, 2500, 2000, 1500, 1000, 500, 250, 200, 100, 50, wherein “n” is an integer equal to 2 or more and especially is any integer from 9 to 100. Particular fragments are those which have contiguous nucleotides of the polynucleotide defined with respect to SEQ ID N°1 or with respect to its complementary sequence (including variant polynucleotides as disclosed herein). In a particular embodiment of the invention, the fragment has at least 9 nucleotides and is in particular a fragment having 9 to 3000 or 9 to 1500 nucleotides or 9 to 1200 nucleotides, or where appropriate has a size of at least 12 or at least 15 nucleotides or more, and is especially in the range or 12 (or 15) to 3000, 12 (or 15) to1500, 12 (or 15) to 1200 nucleotides or 12 (or 15) to 500 nucleotides or 12 (or 15) to 100 nucleotides. All the proposed size limits given herein or ranges may be combined to illustrate other encompassed ranges of sizes for said fragments. The selection of the fragments may be based on the combination of features related to its size and functional features or properties, considered with respect to foreseen applications.

Polynucleotides which are fragments derived from the genome of an IPPy virus according to the invention are especially defined with respect to the polynucleotide having SEQ ID N°1 or with respect to the polynucleotide having SEQ ID N°82 or with respect to a complementary sequence thereof, including with respect to the defined polynucleotide variants of these sequences.

A particular polynucleotide of the invention is one which comprises or which consists in an Open Reading Frame (ORF).

As in already known polyomaviruses, the inventors have established that the genome of the IPPyV comprises Open Reading Frames encoding various polypeptides including the capsid proteins, VP1, VP2 and VP2, the large T antigen, the small T antigen. Particular examples of polynucleotides encoding these capsid proteins are given herein. Other examples obtainable from variant IPPy viruses may be localized on the sequence of the virus genome by comparison with the positions defined in Table 2 referring to the sequence of FIG. 3. Whereas VP1, large T antigen and small T antigen encoding ORFs are on the plus-strand of the genome, ORFs encoding the partially overlapping VP1, VP2 and VP3 proteins are on the minus-strand of the genome.

The invention concerns in particular a polynucleotide selected in the group consisting of: the nucleic acid having sequence disclosed as SEQ ID N°85, the nucleic acid having sequence disclosed as SEQ ID N°86, the nucleic acid having sequence disclosed as SEQ ID N°87, the nucleic acid having sequence disclosed as SEQ ID N°88.

It has been observed by the inventors that the ORF encoding the large T antigen is interrupted by a stop codon.

Accordingly, the invention also concerns a polynucleotide which encodes either of a capsid protein including VP1, VP2 or VP3 or a large T antigen or a small T antigen of a IPPy virus. These capsid proteins or T antigens have been localized by analogy to those of other polyomaviruses, especially with those expressed by the LPV.

In a particular embodiment, the invention relates to a polynucleotide corresponding to an Open Reading Frame selected from the group of:
the polynucleotide comprising or consisting of nucleotides 3733 to 2618 in frame −1 with respect to SEQ ID N°1 (i.e. the ORF is located on the complementary sequence of SEQ ID N°1 and having opposite orientation; the numbering is disclosed with respect to SEQ ID N°1); this ORF encodes the VP1 capsid protein;
the polynucleotide comprising or consisting of nucleotides 4673 to 3615 in frame −3 with respect to SEQ ID N°1 (i.e. the ORF located on the complementary sequence of SEQ ID N°1 and having opposite orientation; the numbering is disclosed with respect to SEQ ID N°1); this ORF encodes the VP2 capsid protein;
the polynucleotide comprising or consisting of nucleotides 4316 to 3615 in frame −3 with respect to SEQ ID N°1 (i.e. the ORF located on the complementary sequence of SEQ ID N°1 and having opposite orientation; the numbering is disclosed with respect to SEQ ID N°1); this ORF encodes the VP3 capsid protein; this ORF encodes the small T antigen;
the polynucleotide comprising or consisting of nucleotides 147 to 716 in frame +3 with respect to SEQ ID N°1 (i.e. the ORF located on the sequence of SEQ ID N°1); this ORF encodes the large T antigen.
the polynucleotide comprising or consisting of nucleotides 147 to 383 and 738 to 2114 in frame +3 with respect to SEQ ID N°1 (i.e. the ORF located on the sequence of SEQ ID N°1);

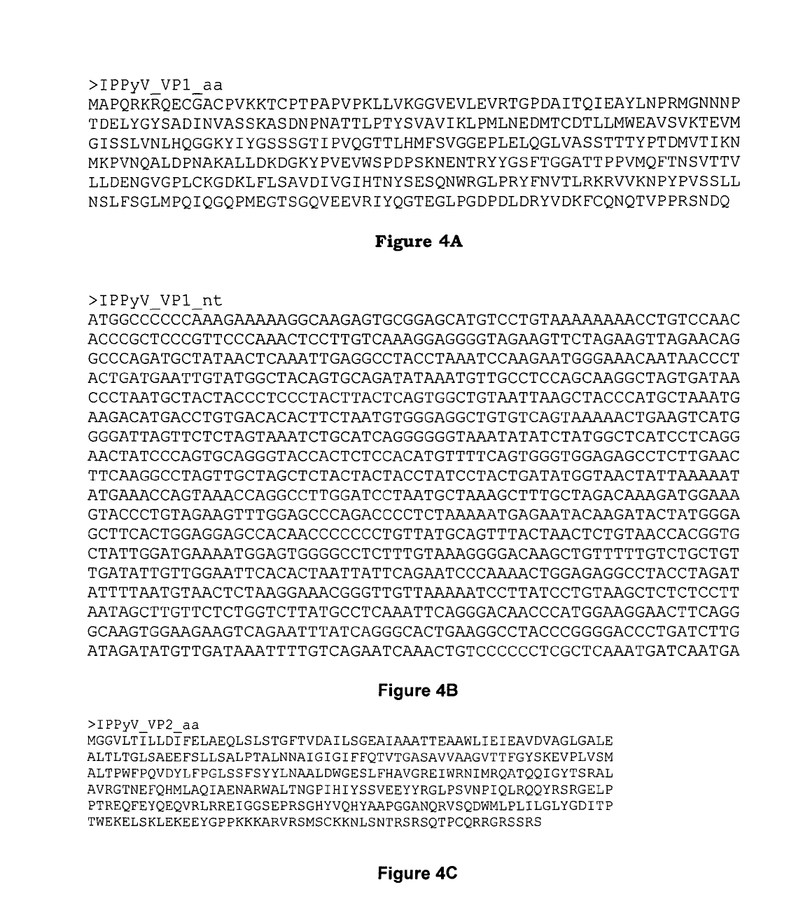

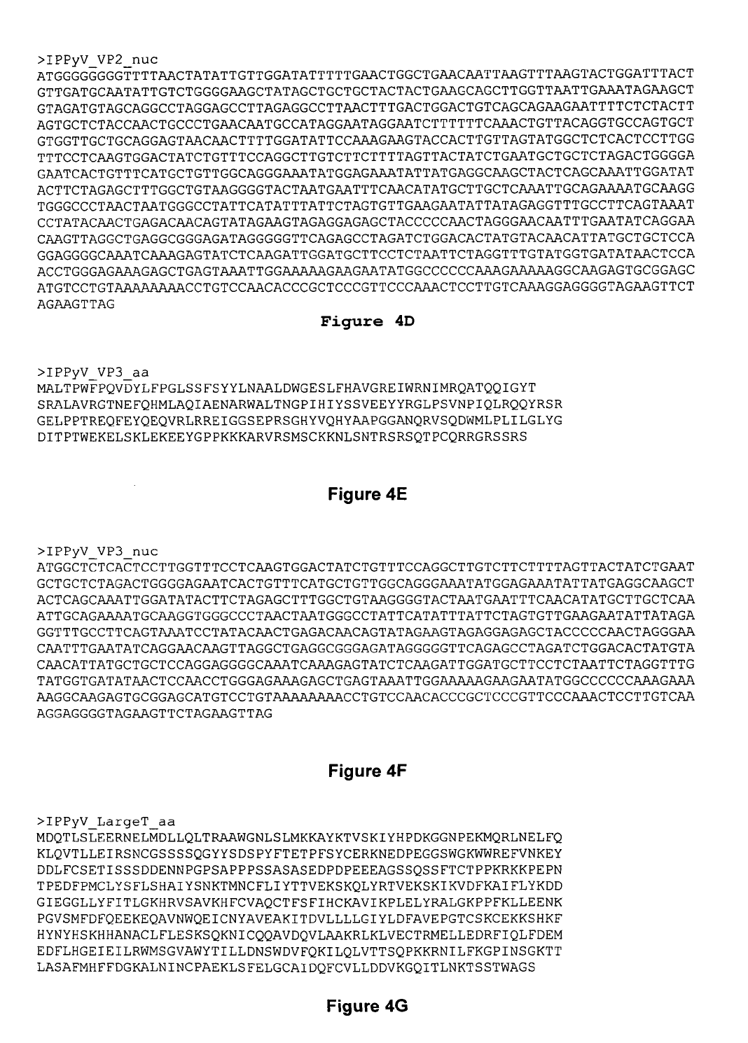

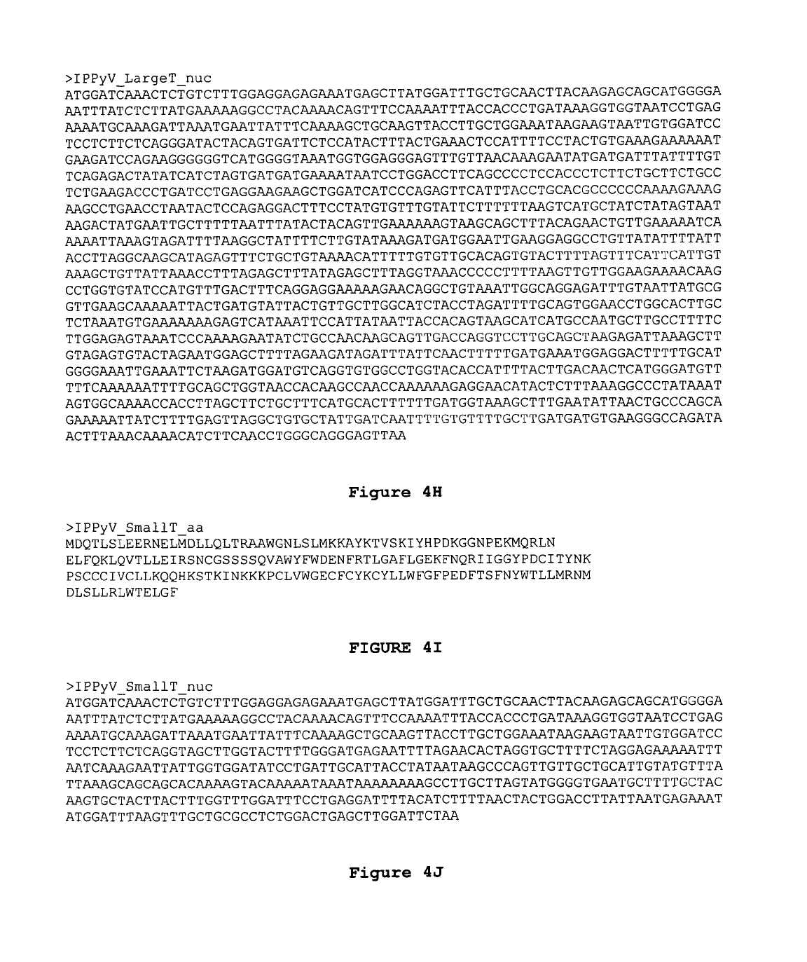

Particular polynucleotides according to the invention are especially derived from the nucleic acid molecule encoding the VP1 or VP2 or VP3 capsid proteins. Such a polynucleotide comprises or has a nucleic acid sequence selected, which comprises or which has a nucleic acid sequence selected from the group of:
the sequence of the VP1 capsid protein disclosed as SEQ ID N° 3 (also in FIG. 4),
the sequence which encodes the VP1 capsid protein, disclosed as SEQ ID N° 2 (also in FIG. 4),
the sequence of the external surface BC loop of VP1 disclosed as SEQ ID NO°13,
the sequence encoding the external surface BC loop of VP1, disclosed as SEQ ID N°12,
the sequence of the external surface DE loop of VP1 disclosed as SEQ ID N°15,
the sequence encoding the external surface DE loop of VP1, disclosed as SEQ ID N°14,
the sequence of the external surface HI loop of VP1 disclosed as SEQ ID N°17, and
the sequence encoding the external surface HI loop of VP1, disclosed as SEQ ID N°16,
the sequence disclosed as SEQ ID N°85 or SEQ ID N°86, SEQ ID N°87 or SEQ ID N°88,
the sequence of the VP2 capsid protein disclosed as SEQ ID N° 5 (also in FIG. 4),
the sequence of the VP3 capsid protein disclosed as SEQ ID N° 7 (also in FIG. 4),
the sequence of the large T antigen disclosed as SEQ ID N°9 (also in FIG. 4),
the sequence of the small T antigen disclosed as SEQ ID N°11 (also in FIG. 4).

The invention also concerns variants of these particular polynucleotides in accordance with the definition which have been given here above. In particular such variants have substituted nucleotides and especially said substitutions are conservative or are phenotypically silent or functionally silent. In another embodiment, the substitutions are non-conservative substitutions and impact on the functional properties of the polynucleotide.

The invention also relates to the polynucleotides defined herein, in association with a heterologous nucleic acid molecule, especially operatively linked with a heterologous nucleic acid, whether coding or non-coding, such as a marker or a tag or non coding sequences such as sequences involved in control of transcription, mRNA processing especially mRNA splicing, polyadenylation, and/or translation, including expression promoters or sequences improving stability.

The heterologous sequence may also or alternatively be suitable for binding the polynucleotide of the invention to a support.

Heterologous sequences associated to the polynucleotide of the invention may be selected for their particular biological activity or may be heterologous nucleic acids coding for protein vectors.

These heterologous sequences may be fused to the polynucleotide of the invention, especially at the 5′ and/or 3′ extremities. It may alternatively be inserted within the polynucleotide in frame with the latter.

As disclosed above the polynucleotides of the invention may be prepared by various methods known from the skilled person. In a particular embodiment of the invention, the polynucleotide is obtainable as a result of:
being amplified and in particular being the product of PCR amplification of DNA extracted from a biological sample of a human host; cutaneous swabs sample previously obtained from a patient with Merkel cell carcinoma have shown to constitute adequate material to isolate the polynucleotide of the invention, or
being a polynucleotide derived from said amplification product by deletion of the DNA region(s) matching the amplification primer sequence(s), or
being the purification product of a viral particle of a human polyomavirus or,
being produced by synthesis or,
being obtained by cloning.

Primers sequences which are suitable for the amplification of a polynucleotide of the invention are those disclosed in table 1 of the present application. Other primers can nevertheless be designed if appropriate or necessary, depending on the region of the genome to be amplified.

The primer polynucleotides defined in Table 1 are as such part of the invention, when taken individually or taken as a set of primers, in particular as couples of forward and reverse primers according to those disclosed in Table 1.

Purification of the polynucleotide according to the invention can be achieved starting from viral particles or starting from cells infected with the polyomavirus (IPPy) and comprises especially the following steps: amplification, especially PCR amplification, for whole of part of the genome, and insertion of the polynucleotide(s) recovered from amplification, in a vector, in particular in a plasmid, for cloning. Amplification primers can be designed starting from the sequence of the genome disclosed as SEQ ID N°1 and in particular can be primers selected among those described in Table 1.

Specific primers suitable for amplification of a polynucleotide characteristic of a polyomavirus of the invention, are the primers having the sequences of SEQ ID N°85, SEQ ID N°86, SEQ ID N°87 or SEQ ID N°88.

The polynucleotide of the invention may be used in various ways, either to detect and especially to detect after an amplification step, including by nested PCR, sequences of IPPy virus strains or isolates from a biological sample, or to express polypeptide products.

Accordingly polynucleotides of the invention may be inserted in vectors, such as cloning or expression vectors. Genetically engineered vectors of the invention or recombinant vectors may be prepared by insertion of the polynucleotide of the invention in a plasmid, a phage, a cosmid, a viral or a retroviral especially lentiviral vector, either replicative competent or replicative incompetent viral or retroviral, especially lentiviral, vector. In said vector the polynucleotide of the invention, possibly in association with a heterologous nucleic acid, is operatively linked to a promoter sequence and other necessary control sequences for the transcription and expression, including translation.

The polynucleotides of the invention may also be used as amplification primers or as hybridization probes in accordance with well-known techniques in the art. When used as primer or as hybridization probes, these polynucleotides may advantageously be labelled, including enzymatically or radioactively labelled with known radioisotopes.

The invention also concerns a polyomavirus which is infectious for a human host and which comprises in its genome a polynucleotide as defined herein. In a particular embodiment, such a polyomavirus is a virus particle or a set of virus particles purified from a sample previously obtained from a human host, such as a tissue sample or a fluid sample, including skin sample or a skin swabs sample or a blood sample or a serum sample or which is purified from a tissue culture.

Other tissue or biological samples including tumor samples can be used to obtain the IPPyV.

A particular polyomavirus of the invention is capable of infecting a human host and is characterized in that it encodes a VP1, and/or VP2, and/or VP3 capsid protein and/or a large T antigen, and/or a small T antigen, wherein said VP1 capsid protein:
comprises at least one external surface loop among the group of said loops having amino acid sequences disclosed as SEQ ID N°13 (BC loop of VP1), SEQ ID N° 15 (DE loop of VP1) and SEQ ID N°17 (HI loop of VP1); or
has the amino acid sequence of SEQ ID N° 3 (FIG. 4) or is a variant of said VP1 capsid protein with SEQ ID N° 3 having an amino acid sequence with more that 87.1% identity with the sequence of SEQ ID N° 3 or
is a variant of said VP1 protein having the amino acid sequence of SEQ ID N° 3, wherein said variants immulogically reacts with antibodies raised against the VP1 capsid protein having the amino acid sequence disclosed as SEQ ID N° 3; and especially that preferentially reacts with these antibodies than with antibodies raised against the corresponding polypeptide of an LPV;
   and said VP2 capsid protein:
has the amino acid sequence of SEQ ID N° 5 (FIG. 4) or is a variant of said VP2 capsid protein with SEQ ID N° 5 having an amino acid sequence with at least 74.9% identity with the sequence of SEQ ID N° 5 or
is a variant of said VP2 protein having the amino acid sequence of SEQ ID N°5, wherein said variants immulogically reacts with antibodies raised against the VP2 capsid protein having the amino acid sequence disclosed as SEQ ID N°5; and especially that preferentially reacts with these antibodies than with antibodies raised against the corresponding polypeptide of an LPV;
   and said VP3 capsid protein:
has the amino acid sequence of SEQ ID N° 7 (FIG. 4) or is a variant of said VP3 capsid protein with SEQ ID N° 7 having an amino acid sequence with at least 72.5% identity with the sequence of SEQ ID N° 7 or
is a variant of said VP3 protein having the amino acid sequence of SEQ ID N° 7, wherein said variants immulogically reacts with antibodies raised against the VP3 capsid protein having the amino acid sequence disclosed as SEQ ID N°7; and especially that preferentially reacts with these antibodies than with antibodies raised against the corresponding polypeptide of an LPV;
   and said large T antigen:
has the amino acid sequence of SEQ ID N° 9 (FIG. 4) or is a variant of said large T protein with SEQ ID N° 9 having an amino acid sequence with at least 61.3% identity with the sequence of SEQ ID N° 9 or
is a variant of said large T protein having the amino acid sequence of SEQ ID N°9, wherein said variants immulogically reacts with antibodies raised against the large T antigen having the amino acid sequence disclosed as SEQ ID N°9; and especially that preferentially reacts with these antibodies than with antibodies raised against the corresponding polypeptide of an LPV; or
has the amino acid sequence of SEQ ID N°84;
   and said small T antigen:
has the amino acid sequence of SEQ ID N° 11 (FIG. 4) or is a variant of said small T protein with SEQ ID N° 11 having an amino acid sequence with at least 81% identity with the sequence of SEQ ID N° 11 or
is a variant of said small T protein having the amino acid sequence of SEQ ID N° 11, wherein said variants immulogically reacts with antibodies raised against the small T antigen having the amino acid sequence disclosed as SEQ ID N°11; and especially that preferentially reacts with these antibodies than with antibodies raised against the corresponding polypeptide of an LPV.

According to another embodiment polyomavirus of the invention is characterized in that it
immunologically reacts with antibodies raised against the VP1 capsid protein as defined herein, wherein said VP1 capsid protein is optionally a pentameric capsomer of VP1 protein; or
immunologically reacts with antibodies raised against one of the external surface loops of the VP1 capsid protein having amino acid sequences disclosed as SEQ ID N°13, SEQ ID N° 15 and/or SEQ ID N°17;
it elicits antibodies that are preferentially recognized by polypeptides encoded by an IPPyV nucleic acid, especially ORF, disclosed herein, especially the ORF encoding VP1, than by antibodies contained in a sample infected with a LPV or antibodies raised against LPV polypeptides.

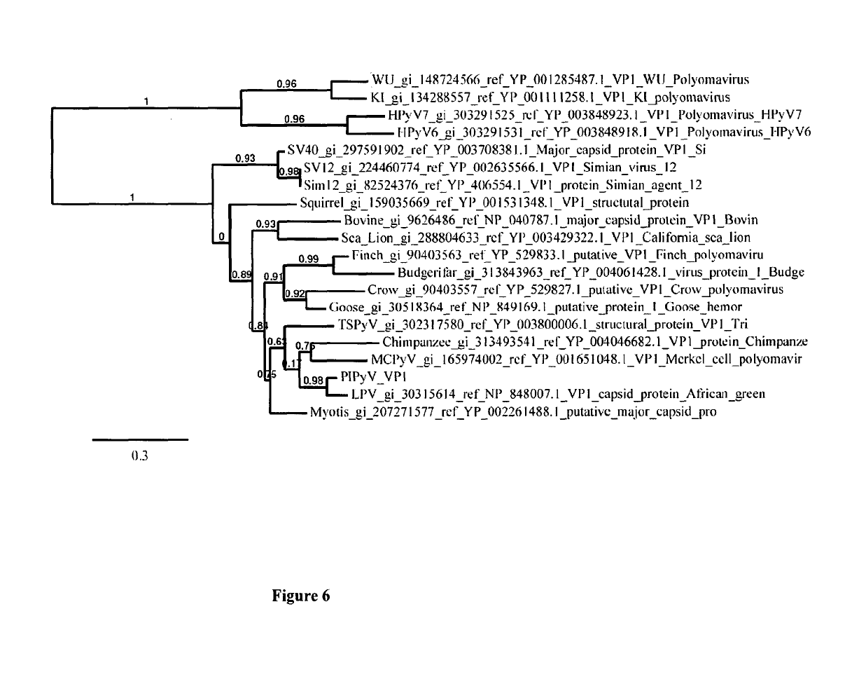

From the comparisons performed by the inventors with other previously identified polyomaviruses, especially from LPV, a phylogenetic three has been achieved, showing that IPPyV is closer to LPV than to other polyomaviruses when comparison is based on nucleotide and amino acid alignments with the VP1 sequence from the LPV (FIG. 6).

Hence, in a particular embodiment, the IPPy virus of the invention is characterized by a sequence identity in the VP1, VP2, VP3, large T antigen, small T antigen, in corresponding nucleic acid sequence or amino acid sequence of respectively 44.9% and 87.1% or more (for VP1 nucleotide sequence aligned on its whole length with the compared sequences), respectively 77.5% and 74.9% or more (for VP2), respectively 75.5% and 72.5% or more (for VP3 aligned on its whole length with the compared sequences), respectively 60.2% and 61.3% or more (for large T antigen when aligned on its whole length of the compared sequences), respectively 78.6% and 81% or more (for small T antigen aligned on its whole length with the compared sequences).

The present invention also concerns polypeptides and in particular concerns a polypeptide which is the product of the expression of a gene or of an ORF polyomavirus as disclosed herein or a polypeptides as defined herein in the context of the description of the virus and its polynucleotides.

Particular polypeptides of the invention are those of the capsid proteins such as VP1, VP2 or VP3 or those of the large T or small T antigens. Particular polypeptides of the invention are those encoded by a polynucleotide defined herein.

In particular, the invention relates to a polypeptide which is chosen from the group of:
(i) a polypeptide encoded by a polynucleotide having a nucleic acid sequence disclosed as SEQ ID N° 2, SEQ ID N°4, SEQ ID N°6, or SEQ ID N°8, SEQ ID N°10, SEQ ID N°12, SEQ ID N°14, SEQ ID N°16;
(ii) a polypeptide comprising or consisting of a molecule having an amino acid sequence disclosed as SEQ ID N°3, SEQ ID N°5, SEQ ID N° 7 or SEQ ID N° 9, SEQ ID N°11, SEQ ID N°13, SEQ ID N°15, SEQ ID N°17, SEQ ID N°84;
(iii) a polypeptide which is a polypeptide variant that immunologically reacts with antibodies raised against a polypeptide comprising or consisting of a molecule having an amino acid sequence disclosed as SEQ ID N°3, SEQ ID N°5, SEQ ID N° 7 or SEQ ID N° 9, SEQ ID N°11, SEQ ID N°13, SEQ ID N°15, SEQ ID N°17, SEQ ID N°84 and in particular a polypeptide variant that reacts with said antibodies preferentially than with antibodies raised against a corresponding polypeptide of an LPV;
(iv) a polypeptide of (i), (ii) or (iii) which comprises or consists of an epitope capable of eliciting an immune response in a human host, especially a humoral or a cellular immune response;
(v) a polypeptide which is a fragment of a polypeptide of (ii) or (iii) and which has an amino acid sequence with an identity with its corresponding aligned sequence in one of the sequences SEQ ID N°3, SEQ ID N°5, SEQ ID N° 7 or SEQ ID N° 9, SEQ ID N°11, SEQ ID N°13, SEQ ID N°15, SEQ ID N°17, SEQ ID N°84 of at least 50%, 60%, 70%, 75%, 80%, 85%, 90%, 95%; and
(vi) a polypeptide of (i), (ii), (iii), (iv), or (v) which is detected in a human host by antibodies raised against the VP1 capsid protein, optionally used as a pentameric capsomer of VP1 protein or by antibodies raised against one of the external surface loops of the VP1 capsid protein having amino acid sequences disclosed as SEQ ID N° 13, SEQ ID N°15, and/or SEQ ID N° 17.

The polypeptides of the invention have advantageously antigenic properties or immunogenic properties and can be expressed as single polypeptides or rather organized as complex structures such as pentamers or capsomers involving the VP1 capsid protein.

The invention also relates to polypeptides (also designated as antigens) which are fragments of the proteins encoded by the ORF of an IPPy virus. Such fragments may have a size of 5 amino acid residues or more, said residues being contiguous in one of the amino acid sequences encoded by said ORF. Particular polypeptides have a size of 5, 6, 7, 8, 9, 10, 15, 18, 19, 20, 25, 30, 40, 50, 100, 200 amino acid residues or more. The maximum length of the polypeptide may depend on the intended use for this polypeptide. In a particular embodiment the size of the polypeptide may be up to 200 or 250 amino acid residues.

The invention also relates to mixtures of polypeptides of the invention such as mixtures of external loops (BC, DE and HI) of VP1.

Other mixtures of polypeptides are formed with polypeptide variants of a given protein encoded by an ORF or mixtures of various polypeptides (including their fragments) within the group of VP1, VP2, VP3, Large T and Small T antigens.

The invention also concerns recombinant polypeptides which are expressed as chimeric polypeptides encompassing sequences from two or more polypeptides (including fragments) derived from VP1, VP2, VP3, Large T and Small T antigens. In particular, the invention relates to fusion polypeptides obtained by recombination of distinct antigens.

The particular external surface loops of the VP1 capsid protein are interesting as a signature of the IPPy virus, and have significantly differences in their composition with corresponding regions in other human polyomaviruses. Accordingly, they constitute antigenic or immunogenic polypeptides which enable specific determination of the presence of the human IPPy virus with respect to other known human polyomaviruses. By “specific” it is intended that the polypeptides are preferentially recognized by antibodies raised against IPPyV polypeptides than with antibodies raised against polypeptides of other polyomaviruses, or their expression products, especially of LPV, or especially of other human polyomaviruses. In a particular embodiment, specific determination means that no significant cross-reaction with polypeptides of other human polyomarivuses is expected in usual conditions for detection assays, such as ELISA.

In a particular embodiment, the polynucleotides of a LPV and especially the ORFs encoding the VP1, VP2, VP3, Large T and Small T of LPV and the polypeptides encoded by said polynucleotides, and in particular the VP1, VP2, VP3, Large T and Small T polypeptides of LPV are not as such part of the invention.

The invention also concerns a kit for the detection of exposure and/or infection by a human polyomavirus of the IPPyV species in a biological sample of a human host, which comprises:
a polypeptide according to the definition provided herein;
optionally means for the detection of an immunological antigen-antibody complex.

The detection can especially be performed by any known techniques available in the art including immunoassays such as Elisa or Western Blot, Elispot, antibodies microarrays, tissue microarrays, coupled to immunochemistry.

The invention thus relates to a method for the detection of exposure to, or infection by an IPPyV, comprising the step of contacting said sample with polypeptide(s) of the invention and detecting the presence of an immunological complex between said polypeptides and antibodies present in the sample.

In a particular embodiment, the detection is performed on a sample previously obtained from a human patient.

The invention relates to a set of polynucleotides characteristic of the nucleic acid encoding VP1, in particular polynucleotides suitable for the detection of a DNA of a IPPyV genomic sequence, especially for the specific detection of said IPPyV DNA thereby excluding detection of genomic sequences of a different human polyomavirus. A particular set of primers is the set of polynucleotides comprising:
a first pair of polynucleotides constituted by a nucleic acid having the sequence disclosed as SEQ ID N°85 and a nucleic acid having the sequence disclosed as SEQ ID N°86 and/or
a second pair of polynucleotides constituting by a nucleic acid having the sequence disclosed as SEQ ID N°87 and a nucleic acid having the sequence disclosed as SEQ ID N°88.

The polynucleotides of said set may vary in size as disclosed in the present application and especially may be variants thereof having 10% to 50% additional nucleotides at their 5′ and 3′ ends or 10% to 30% nucleotides less at their 5′ and/or 3′ ends

According to another embodiment, a kit of the invention for the detection of exposure and/or infection by a human polyomavirus of the IPPyV species in a biological sample of a human host comprises
at least one polynucleotide according to the invention defined herein suitable for use as amplification primer(s) or a set of polynucleotides as defined herein;
optionally means to perform an amplification reaction on DNA;
optionally means to detect amplification products;
optionally means required for DNA extraction;
optionally a control DNA molecule for simultaneous amplification with the amplification primer(s), wherein said control is a polynucleotide according to the invention defined herein which is tagged.

The invention thus relates to a method of detection of an IPPyV as defined herein, comprising a step of amplification, direct hybridization with at least one specific probe to IPPyV nucleic acid and/or sequencing of the said virus using the primers of the invention. In a particular embodiment, amplification, hybridization, or sequencing of nucleic acid using the primers of the invention is indicative of the infection by an IPPyV or of the presence of an IPPyV in the sample. When referring to sequencing the invention concerns detection of the IPPyV in samples previously obtained or screening for IPPyV in biological materials or products, such as with deep sequencing techniques, such as pyrosequencing or illumina sequencing.

Said detection is especially performed on a sample previously obtained in a human patient.

The invention is also aimed at preparing products such as tissue or blood products which are IPPyV-free. Accordingly products in which performing the detection method leads to a result showing no IPPyV or the absence of IPPyV nucleic acid are so-called IPPyV-free.

The invention encompasses IPPyV-free products, and methods to obtain them using the polynucleotides and polypeptides of the invention, for use in human as medical products, such as graft, tissue replacement, blood transfusion, growth factors or coagulation factors products, cell therapy and all biological products manufactured in cell culture (like vaccines, monoclonal antibodies and therapeutic proteins).

The invention also concerns antibodies raised against a polypeptide of the invention, and in particular either monoclonal antibodies or polyclonal serum. The polypeptide of the invention used to obtain these antibodies is used in available techniques of preparation of antibodies including by administration to an animal and recovery of antibodies produced. The invention thus concerns a method of preparation of antibodies comprising the immunization of an animal with a polypeptide as described herein and the recovery of antibodies recognizing IPPyV epitopes and in particular recognizing antigens comprising the polypeptides used for the immunization step.

Monoclonal antibodies may be produced by known techniques such as those involving preparation of hybridoma after immunization of an animal with a polypeptide of the invention followed by obtaining immune cells from the spleen of said immunized animal and fusing said cells with a cancer cell (such as a myeloma cell) to immobilize the immune cell thereby obtaining a hybridoma producing monoclonal antibodies when the hybridoma are cultivated.

Antibodies according to the invention encompass antibody fragments, which have binding capacity for the polypeptide of the invention. Particular fragments are Fab fragments of F(ab′)2 fragments. They may be produced by synthesis or by proteolytic cleavage of full antibodies using enzymes such as papain or pesin respectively. The antibodies may alternatively be single-chain antibodies including variable heavy chain(s) capable of binding antigens.

The antibodies of the invention may be obtained in animals such as rodents, especially mice or rabbits. They may be engineered to provide chimeric antibodies especially humanized antibodies in particular having a constant fragment characteristic of human antibodies. Methods for the preparation of humanized antibodies are well known in the art. The antibodies may be human antibodies.

Particular antibodies according to the invention do not cross react with the following human polyomaviruses: the JC virus, the BK virus, the KI polyomavirus, the WU polyomavirus, the Merkel cell polyomavirus, the Trichodysphasia spinulosia-associated polyomavirus. In a particular embodiment, the antibodies further do not cross-react with the monkey the lymphotropic polyomavirus.

The invention also concerns the use of a polypeptide according to the invention, or the use of a polynucleotide according to the invention, for the in vitro detection of a human host exposed to or sero-positive for a IPPyV, or for monitoring the infection by an IPPyV.

The invention also relates to the use of a polynucleotide or a polypeptide of the invention, in association with a polynucleotide or respectively a polypeptide of a MCPy virus which can be recognized by a serum of a patient infected with a MCPy virus, for the detection of co-exposure or of co-infection by a IPPPy virus and an MCPy virus.

According to another embodiment of the invention, these polynucleotides or polypeptides are used as a marker for tumor or cancer diagnosis or prognosis especially for detection of carcinoma or other cancers.

The invention also relates to the use of a polynucleotide or the use of a polypeptide described in the present application, to assay a biological sample, such as a tissue sample or sample of biological secretion (such as cutaneous swab), a blood or a serum sample or a urine sample, for the presence of IPPyV, genomic sequences or expression products or for the presence of antibodies recognizing said expression products.

In particular such a biological sample may be obtained from a patient who is immunocompromised and especially a patient diagnosed for lymphoproliferative disorders, for example leukemia or leucoencephalopathies, or suspected to be affected by such disorders.

The invention also relates to an immunogenic composition comprising a polypeptide of the invention with a pharmaceutical vehicle and optionally an adjuvant of the humoral response and/or cellular response and optionally a further prophylactic active ingredient or therapeutically active ingredient such as one having antiviral or antitumoral activity.

Pharmaceutical vehicles or carriers include solvents, salts solution, buffers, dispersion compositions, preservative agents, media for delivery especially for control or sustained delivery and any appropriate compound for formulation of a drug.

A particular immunogenic composition is for use in prophylaxis against infection by a human polyomavirus of the IPPyV species or in immunotherapy against the development of an infection by a human polyomavirus of the IPPyV species, especially against the development of the infection toward clinical symptoms, including toward a tumoral pathology.

The invention also concerns a cell or a cell-culture which contains a polynucleotide according to the definition provided herein and/or which expresses a polypeptide according to the definition provided herein. The cell is especially not an embryonic stem cell obtained by a process that would require destruction of embryo.

The invention is also directed to a cell or a cell-culture which is infected by a polyomavirus of the IPPyV species.

The invention also relates to the use of a polynucleotide or a polypeptide of the invention, in association or a polypeptide of the invention, in association with a polynucleotide or respectively a polypeptide of a MC Py virus which can be recognized by a serum of a patient infected with a MCPy virus, for the detection of co-exposure or of co-infection by a IPPy virus and an MCPy virus.

Further properties and advantages of the invention will be apparent from the examples and figures which follow:

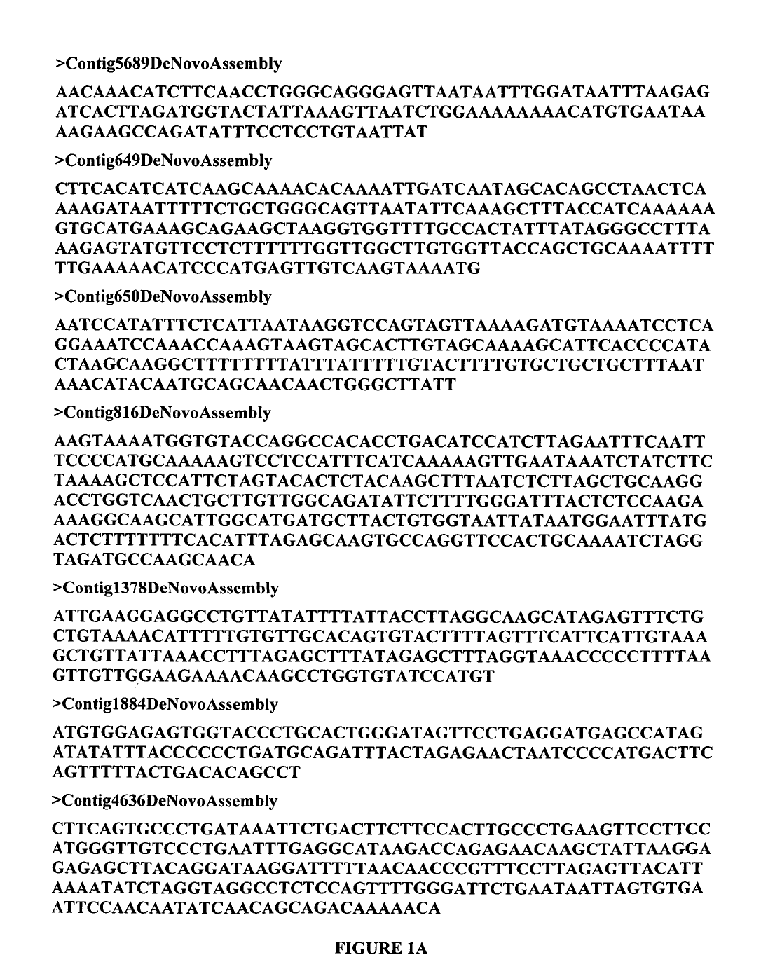

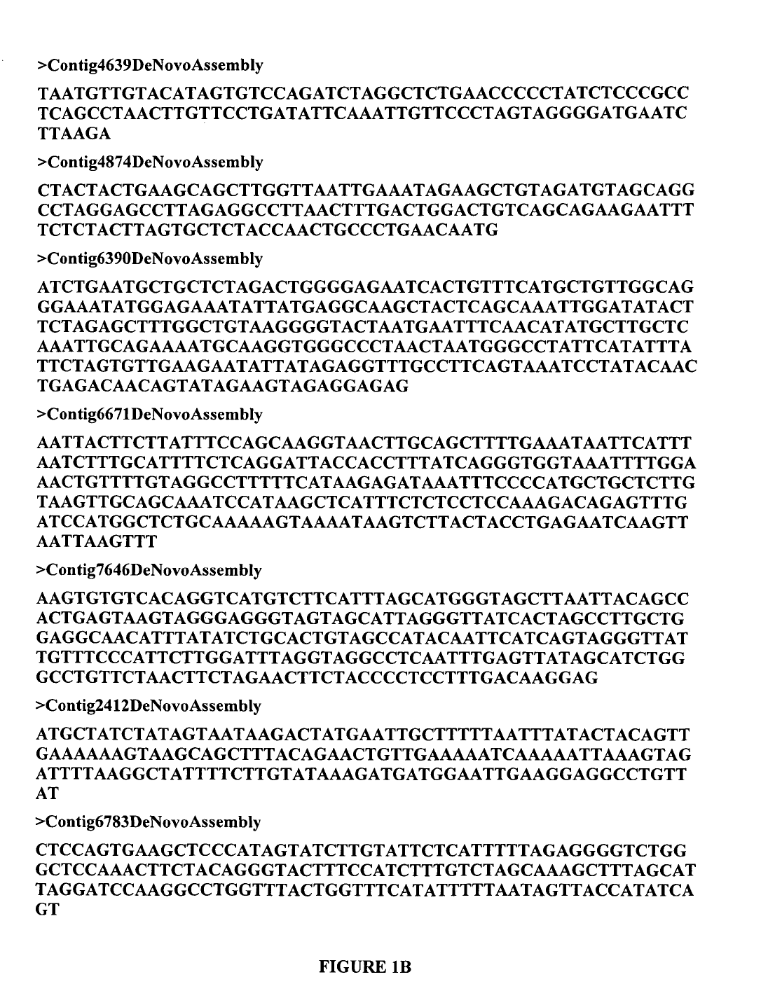

FIG. 1: Mapping of the 14 contigs with the reference sequence of LPV (NCB1 accession: M30540, version M30540.1, GI: 333282 (NCBI Blastn).

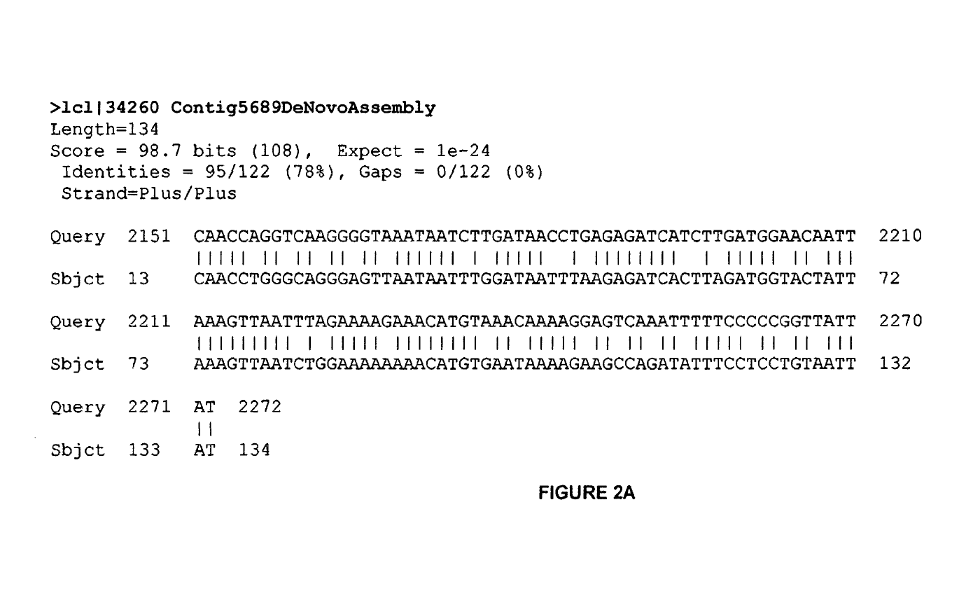

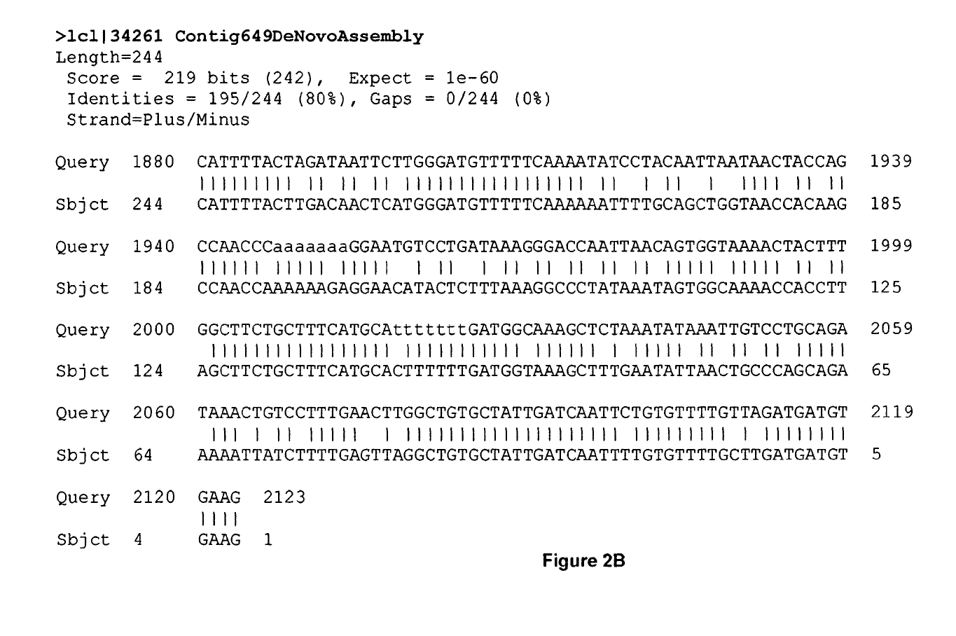

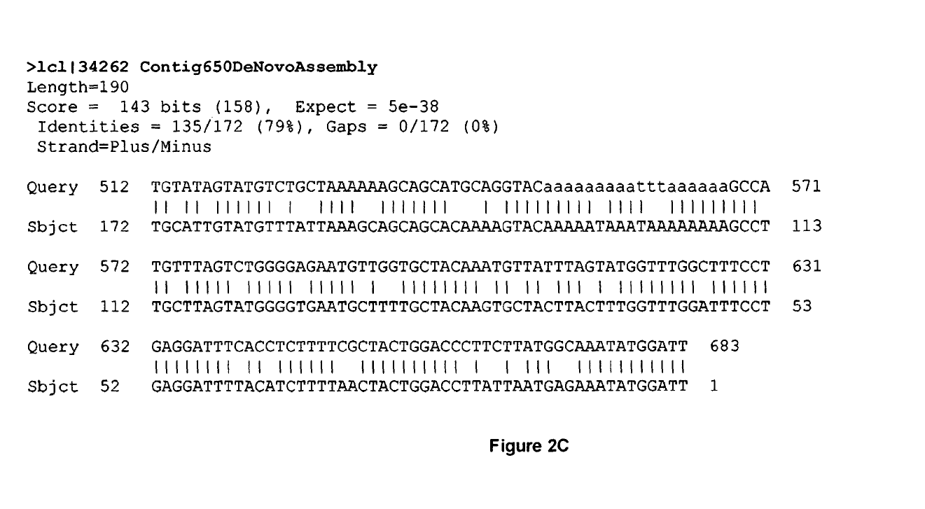

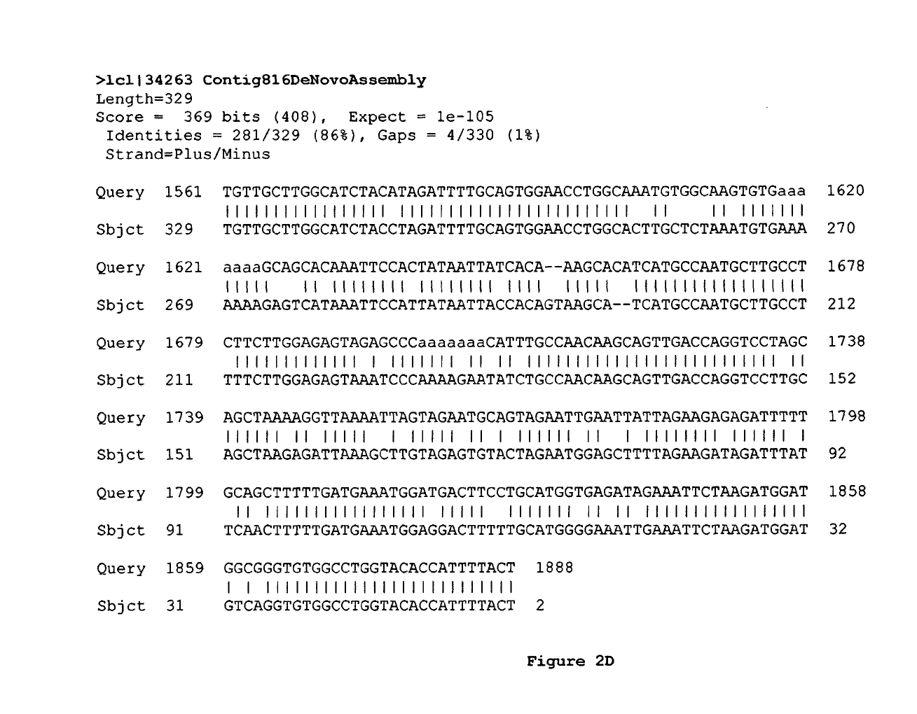

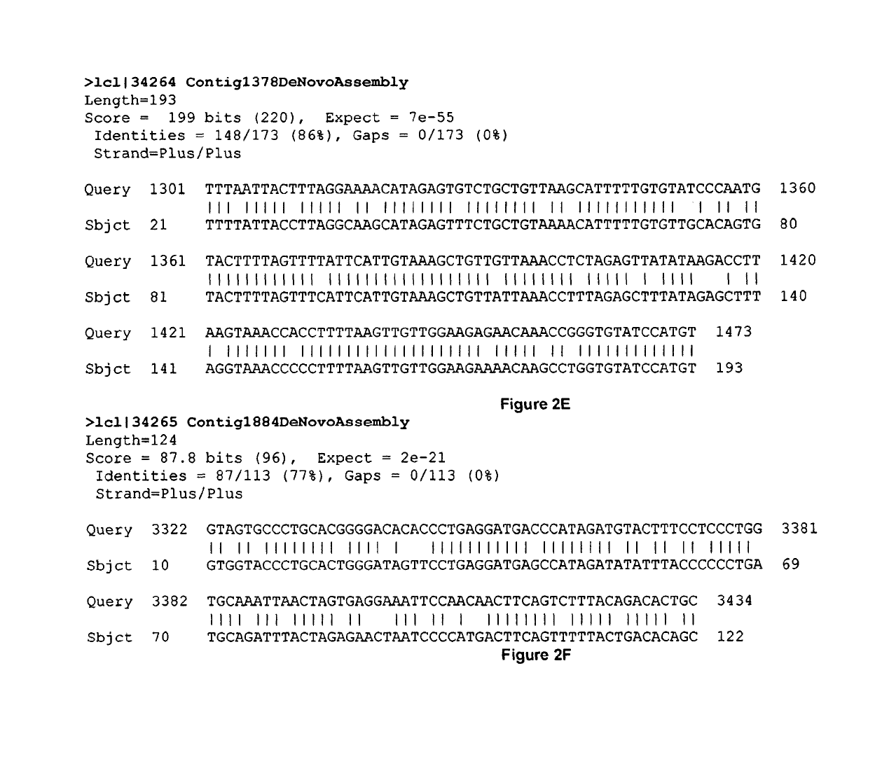

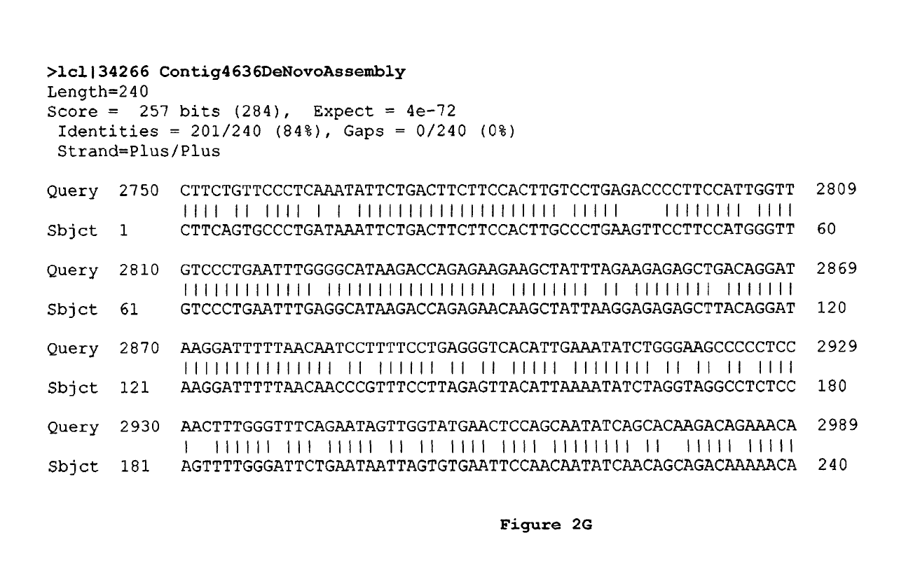

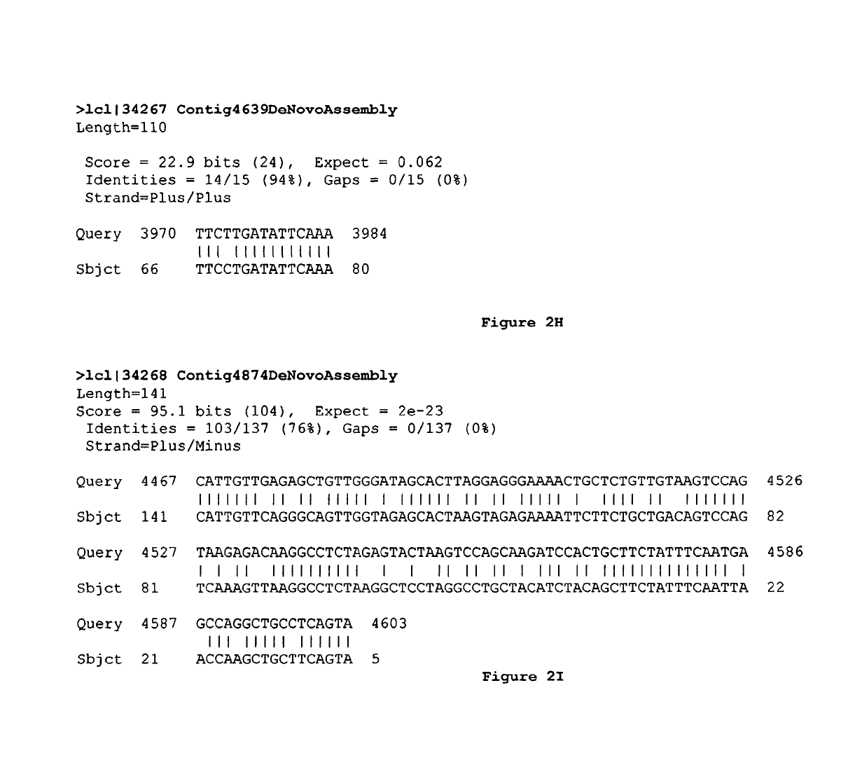

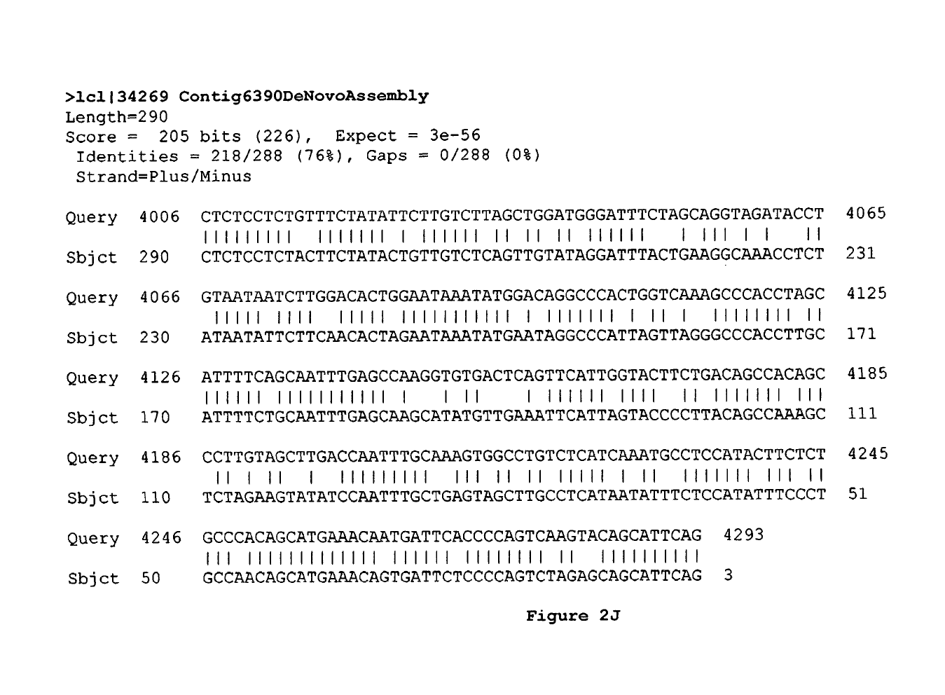

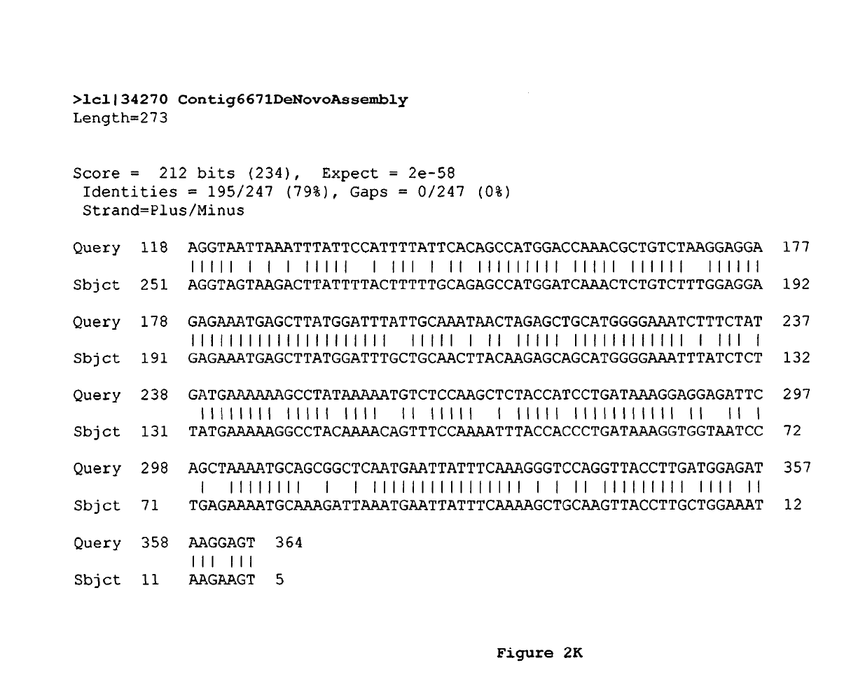

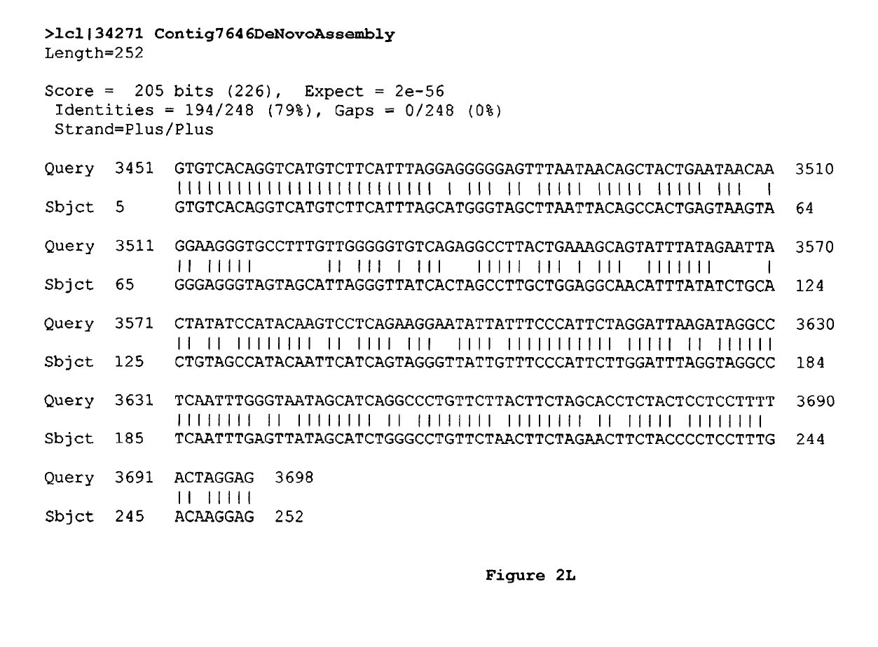

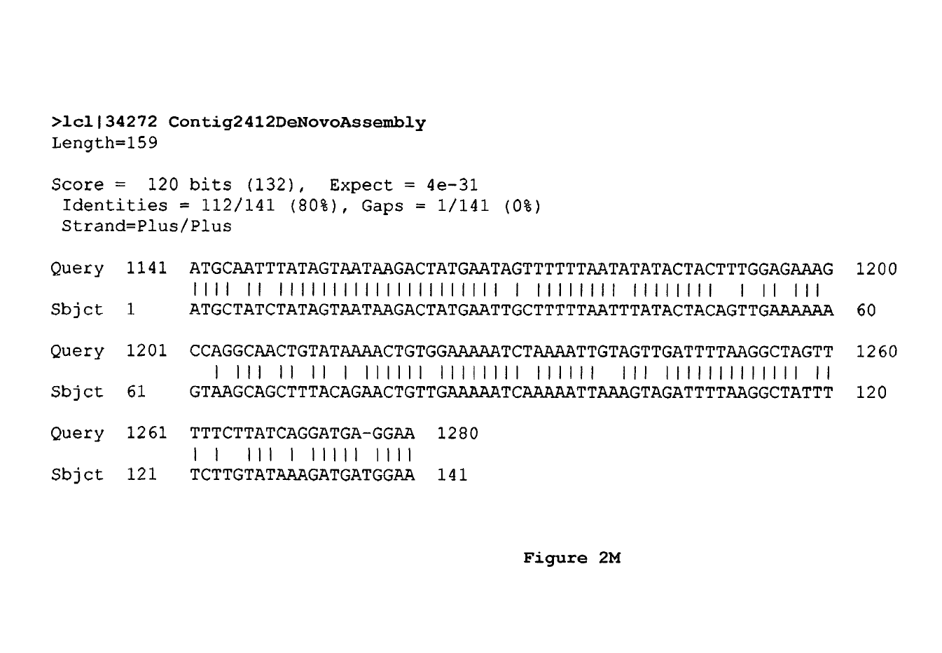

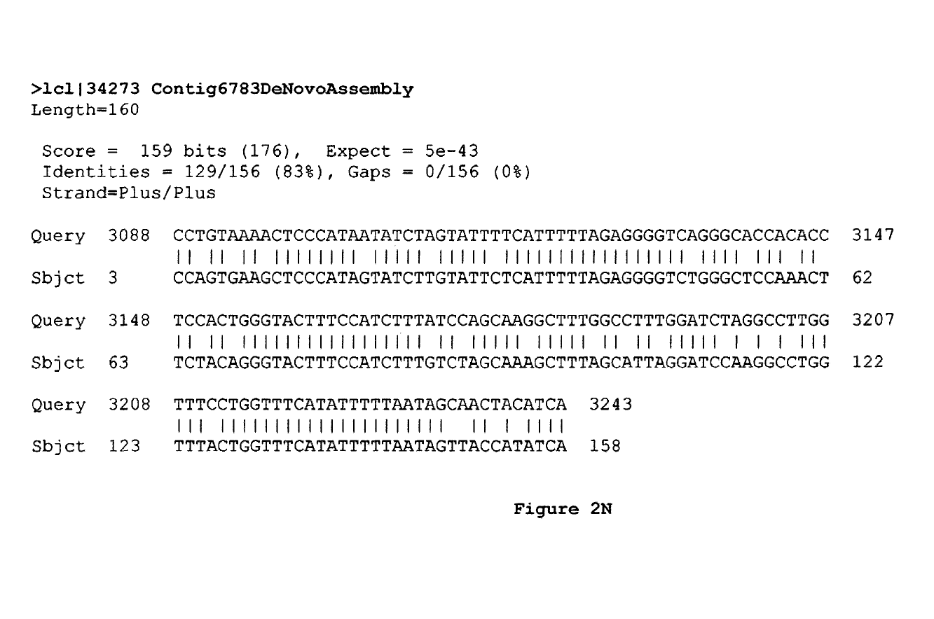

FIG. 2: Alignment of the 14 contigs with the reference sequence of LPV (NCB1 accession: M30540, version M30540.1, GI: 333282 (NCBI Blastn) Query=LPV; Sbjct=IPPyV.

FIG. 3: Nucleotide sequence of a genomic nucleic acid of IPPyV;

FIG. 4: Amino acid and nucleotide sequences of IPPyV sequences of VP1 (FIGS. 4A and 4B), VP2 (FIGS. 4C and 4D), VP3 (FIGS. 4E and 4F), Large T (FIGS. 4G and 4H) and Small T (FIGS. I and 4J).

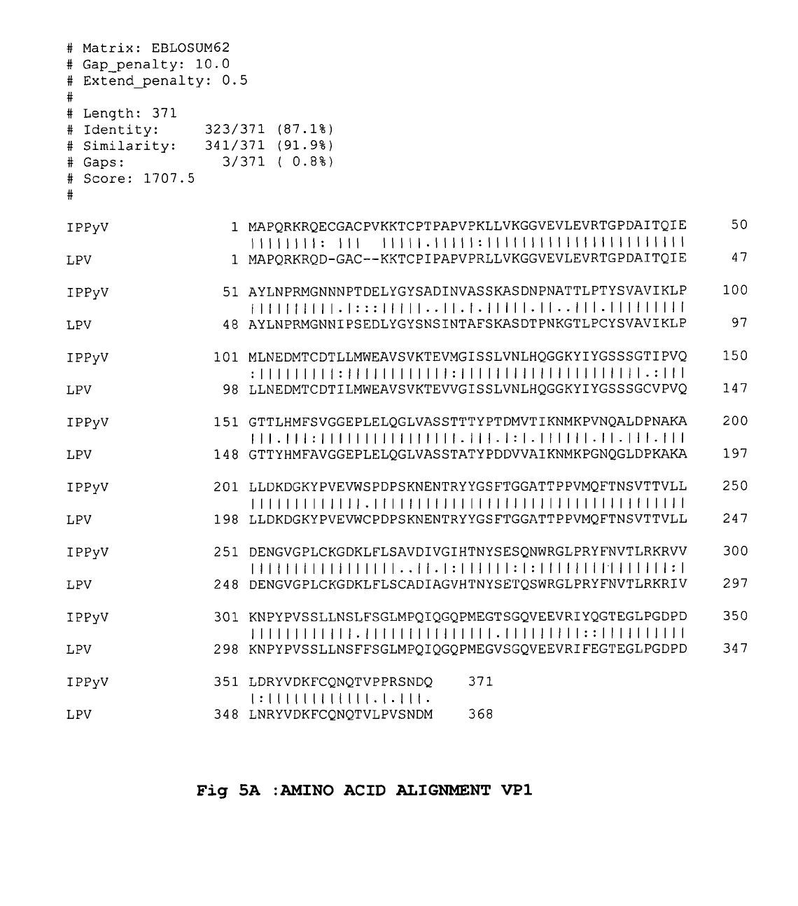

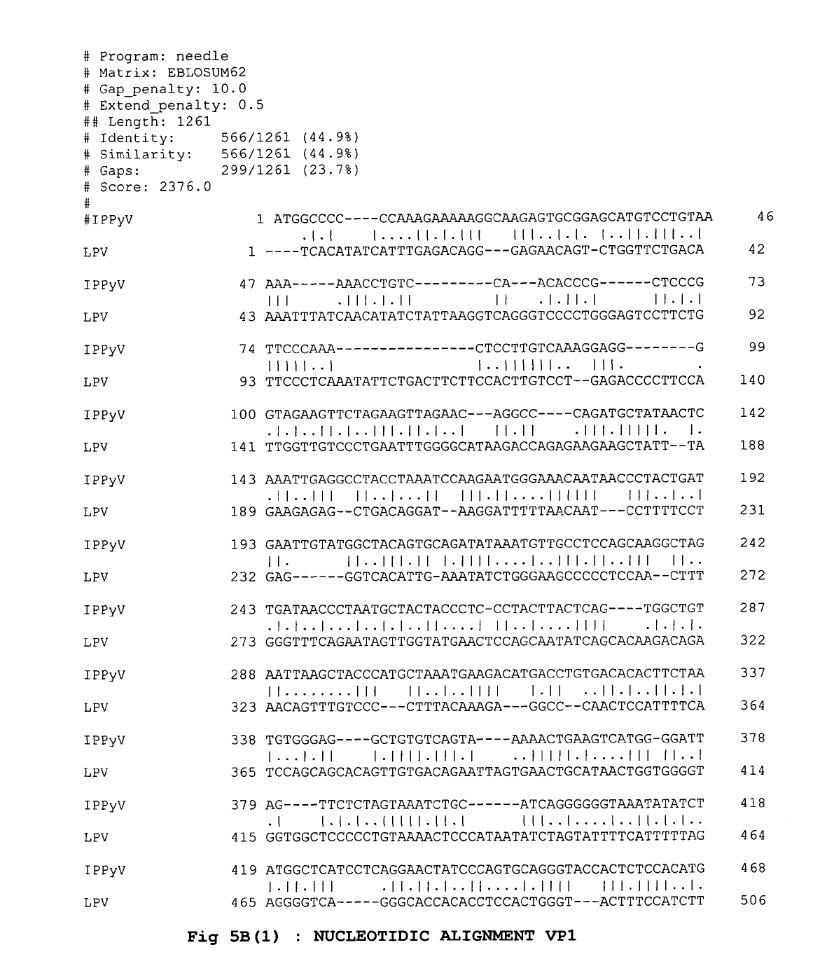

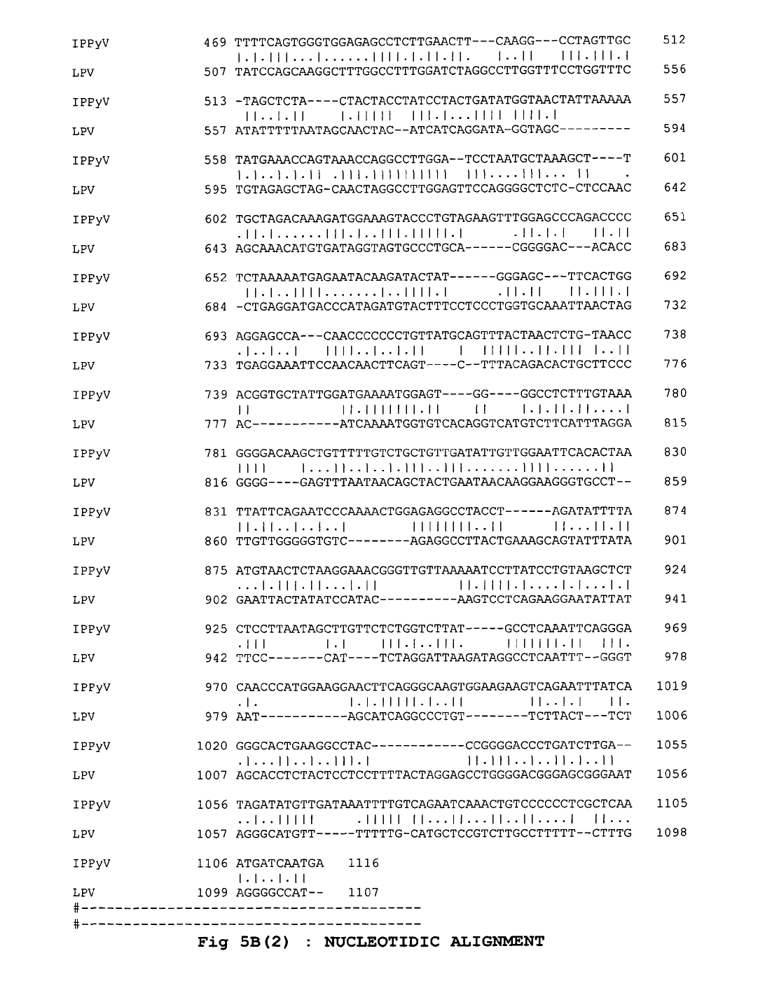

FIG. 5: Nucleic acid alignment of VP1 for IPPyV and LPV

FIG. 6: Phylogenetic tree of the Polyomaviridae based on VP1 amino acid sequences (maximum likelihood.PhymL)

| Reference Polyomavirus genome | Accession number VP1 |
|---|---|
| NC_014743 Chimpanzee polyomavirus, complete genome | YP_004046682.1 |
| NC_001442 Bovine polyomavirus, complete genome | NP_040787.1 |
| NC_014407 Polyomavirus HPyV7, complete genome | YP_003848923.1 |
| NC_014406 Polyomavirus HPyV6, complete genome | YP_003848918.1 |
| NC_013796 California sea lion polyomavirus 1, complete genome | YP_003429322.1 |
| NC_014361 Trichodysplasia spinulosa-associated polyomavirus, | YP_003800006.1 |
| complete genome | |
| NC_001669 Simian virus 40, complete genome | YP_003708381.1 |
| NC_012122 Simian virus 12, complete genome | YP_002635566.1 |
| NC_011310 Myotis polyomavirus VM-2008, complete genome | YP_002261488.1 |
| NC_010277 Merkel cell polyomavirus, complete | YP_001651048.1 |
| NC_009951 Squirrel monkey polyomavirus, complete genome | YP_001531348.1 |
| NC_009539 WU Polyomavirus, complete genome | YP_001285487.1 |
| NC_009238 KI polyomavirus Stockholm 60, complete genome | YP_001111258.1 |
| NC_007923 Finch polyomavirus, complete genome | YP_529833.1 |
| NC_007922 Crow polyomavirus, complete genome | YP_529827.1 |
| NC_007611 Simian agent 12, complete genome | YP_406554.1 |
| NC_004800 Goose hemorrhagic polyomavirus, | NP_849169.1 |
| NC_004764 Budgerigar fledgling disease polyomavirus, complete | YP_004061428.1 |
| genome | |
| NC_004763 African green monkey polyomavirus, complete genome | NP_848007.1 |
| NC_001699 JC polyomavirus, complete genome | NP_848007.1 |
| NC_001663 Hamster polyomavirus, complete genome | NP_056733.1 |
| NC_001538 BK polyomavirus, complete genome | YP_717939.1 |
| NC_001515 Murine polyomavirus, complete genome | NP_041267.1 |
| NC_001505 Murine pneumotropic virus, complete genome | NP_041234.1 |
| NC_013439 Orangutan polyomavirus, complete genome | YP_003264533.1 |

FIG. 7: Aminoacid alignment of VP1 from MCPyV, LPV and IPPyV viruses (Kalign (2.0) alignment in ClustalW format).

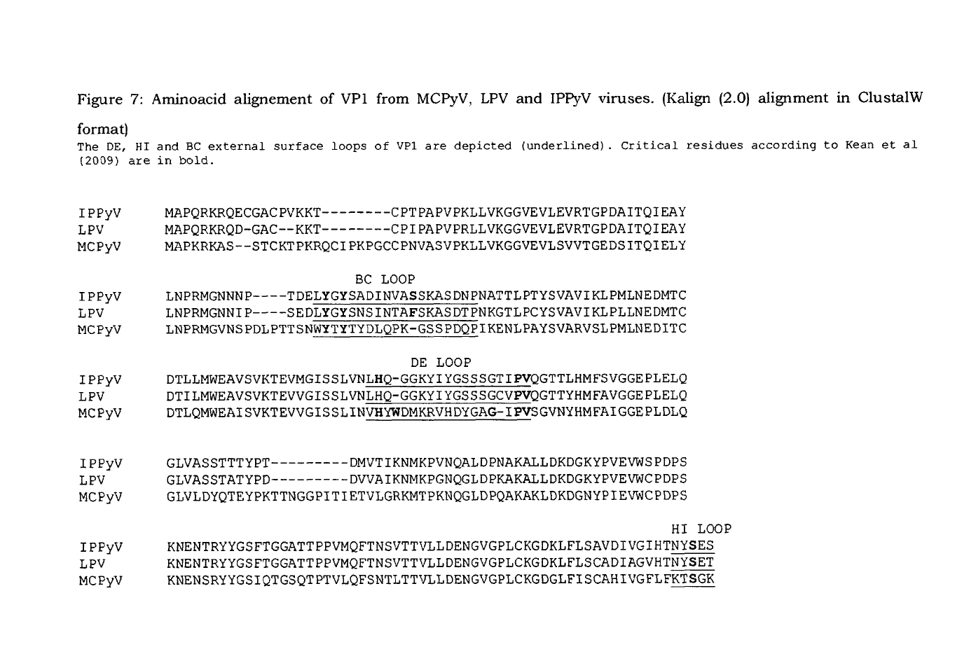

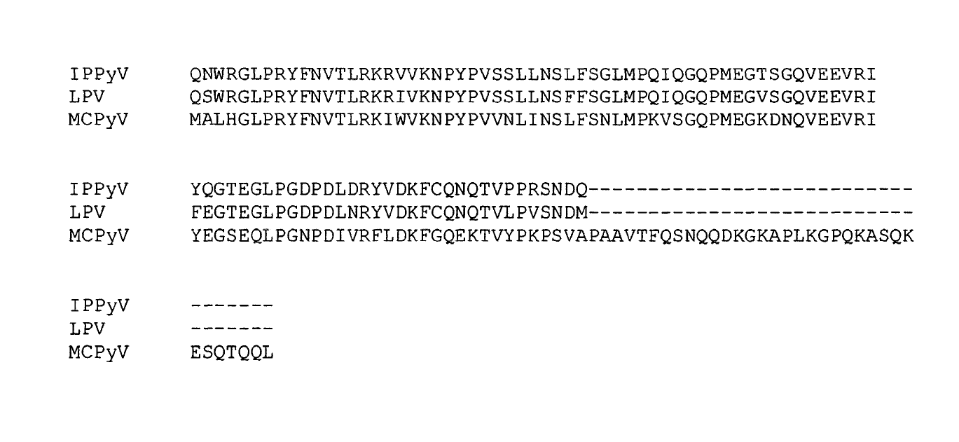

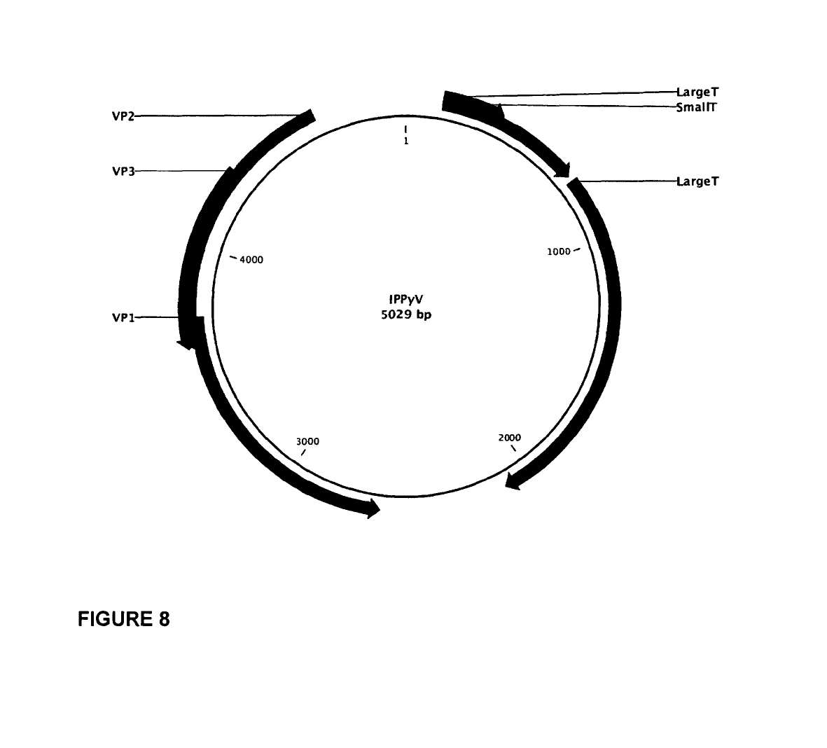

FIG. 8: Schematic representation of the IPPyV genome (see details in table 2)

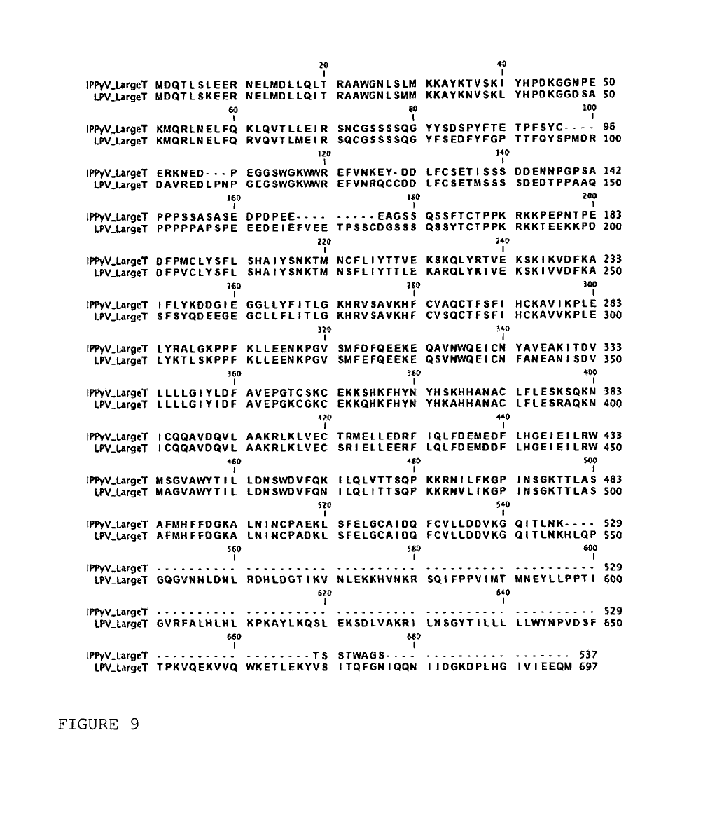

FIG. 9: Alignment between the LPV and IPPyV viruses showing expression of a truncated IPPyV large T protein.

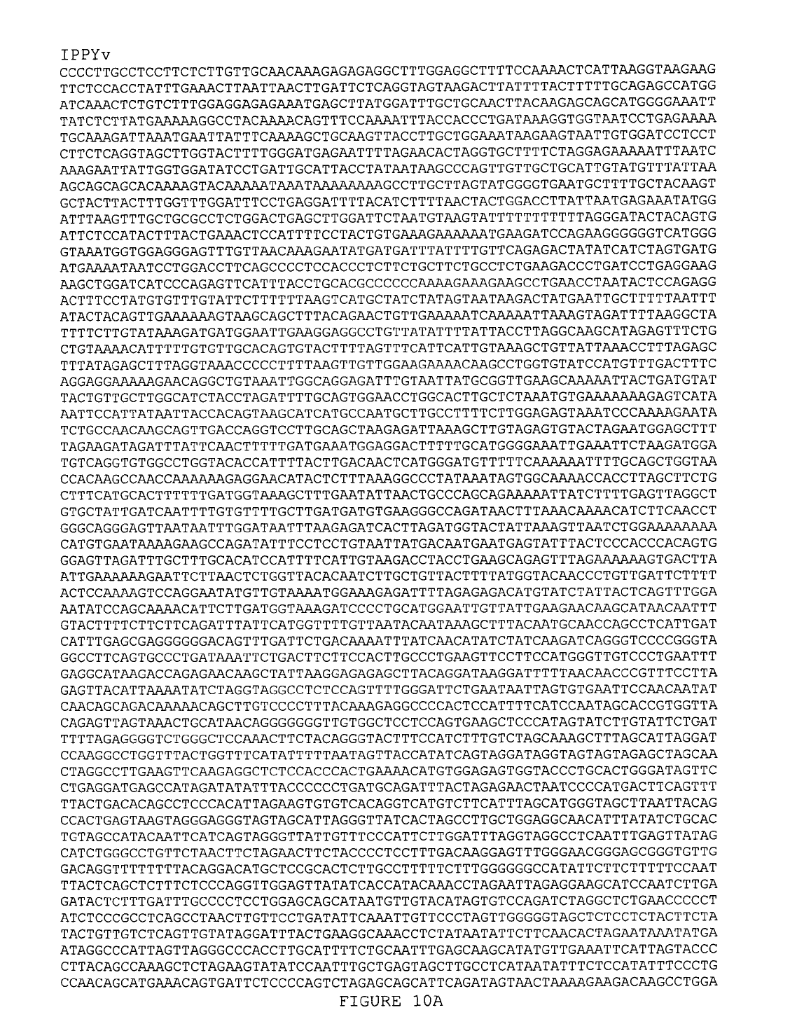

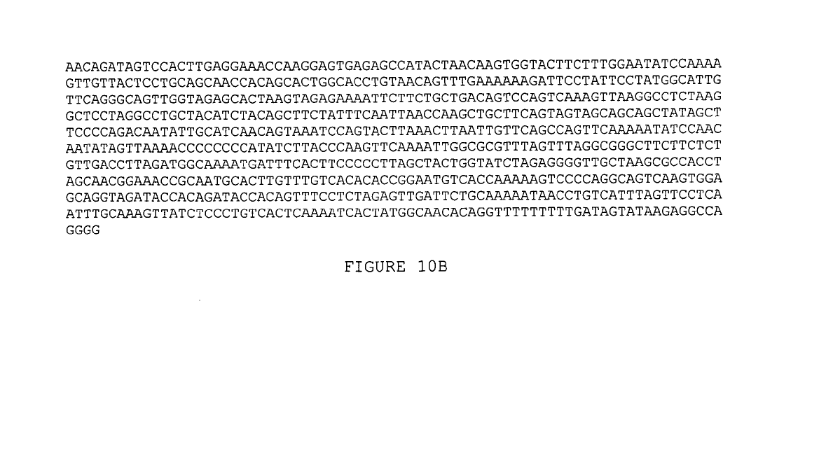

FIG. 10: Nucleotide sequence of a genomic nucleic acid (SEQ ID N°82)

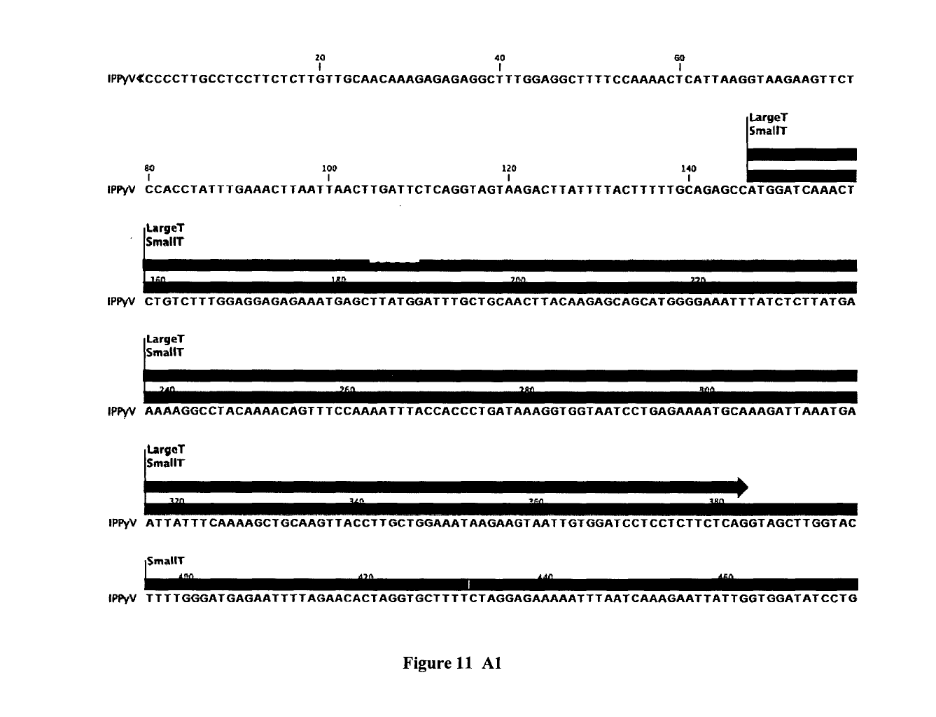

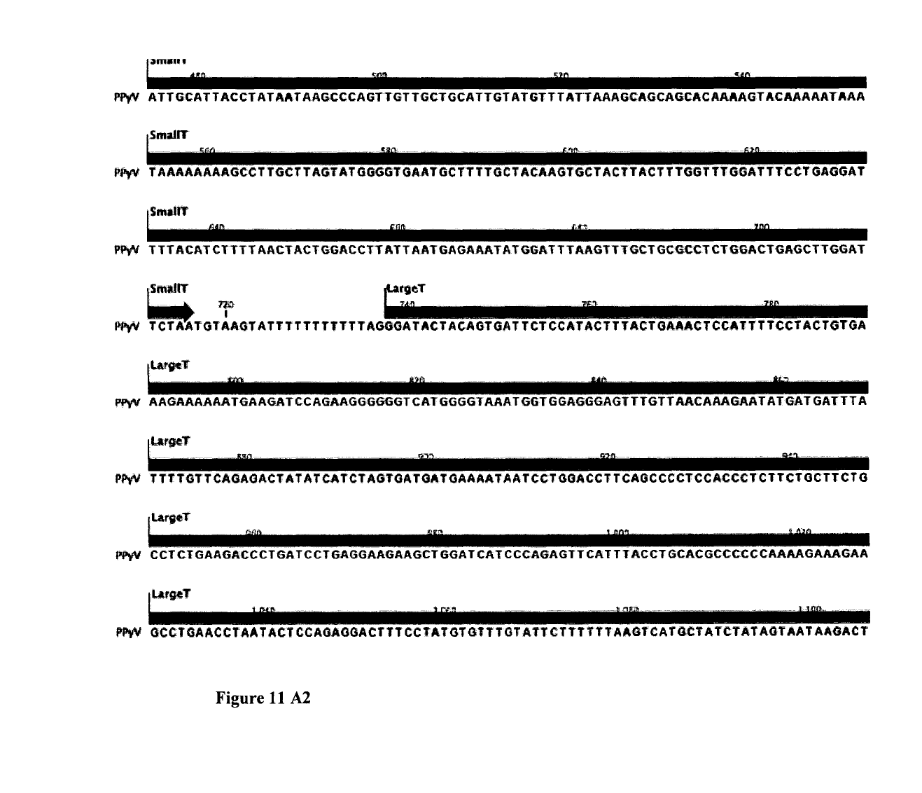

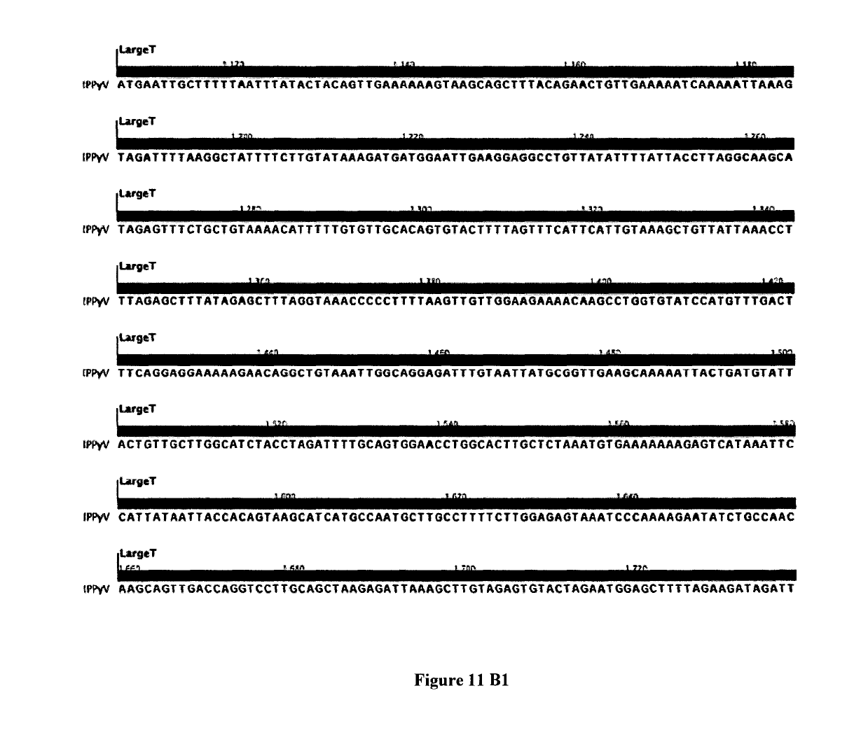

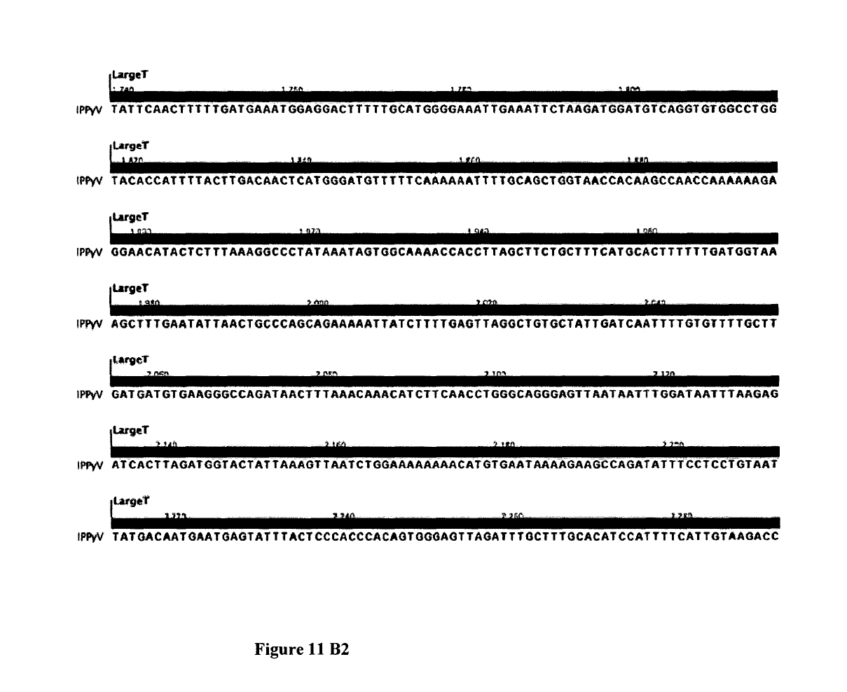

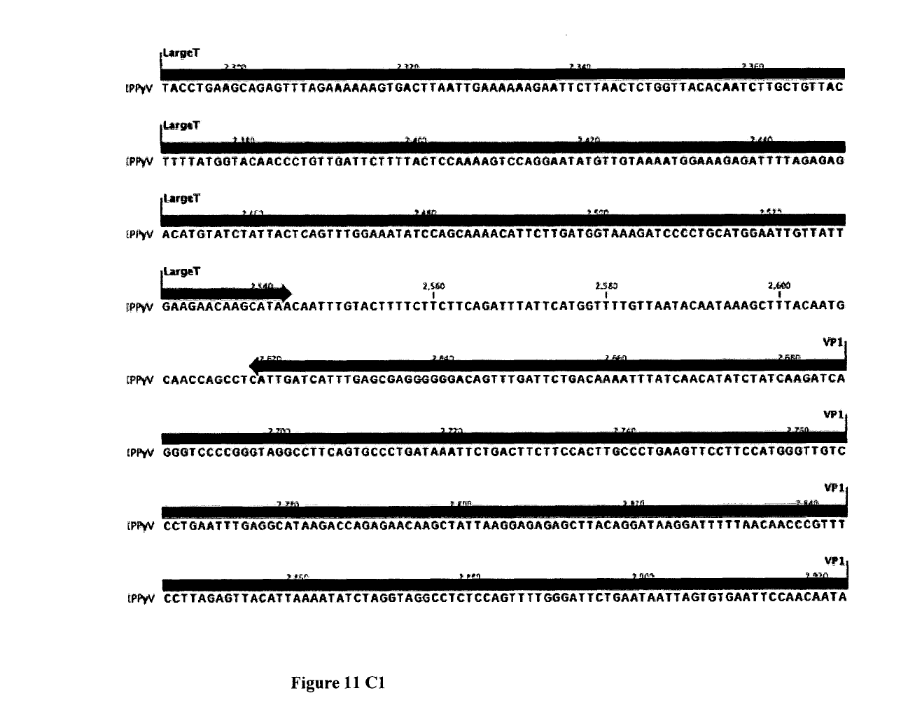

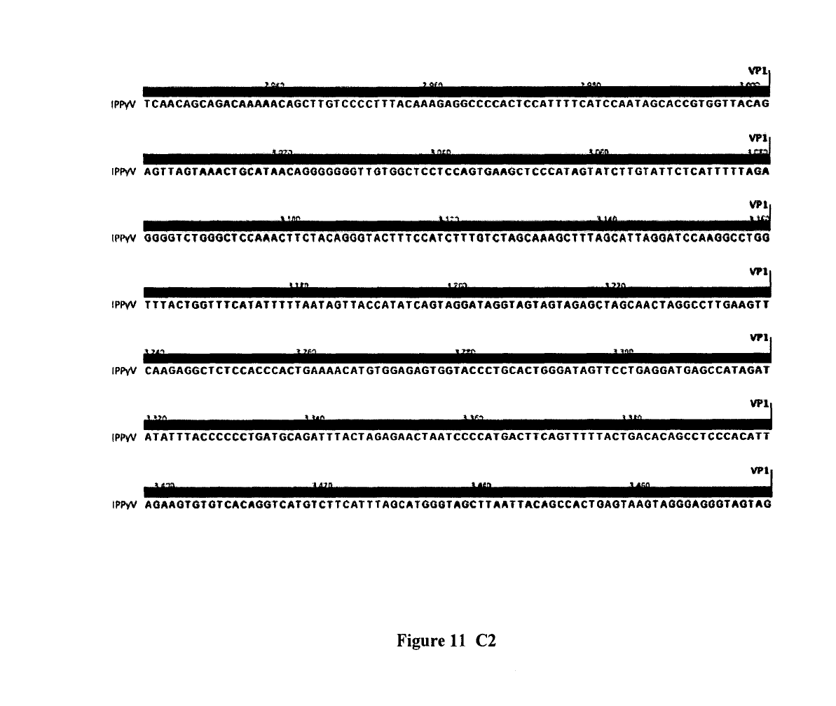

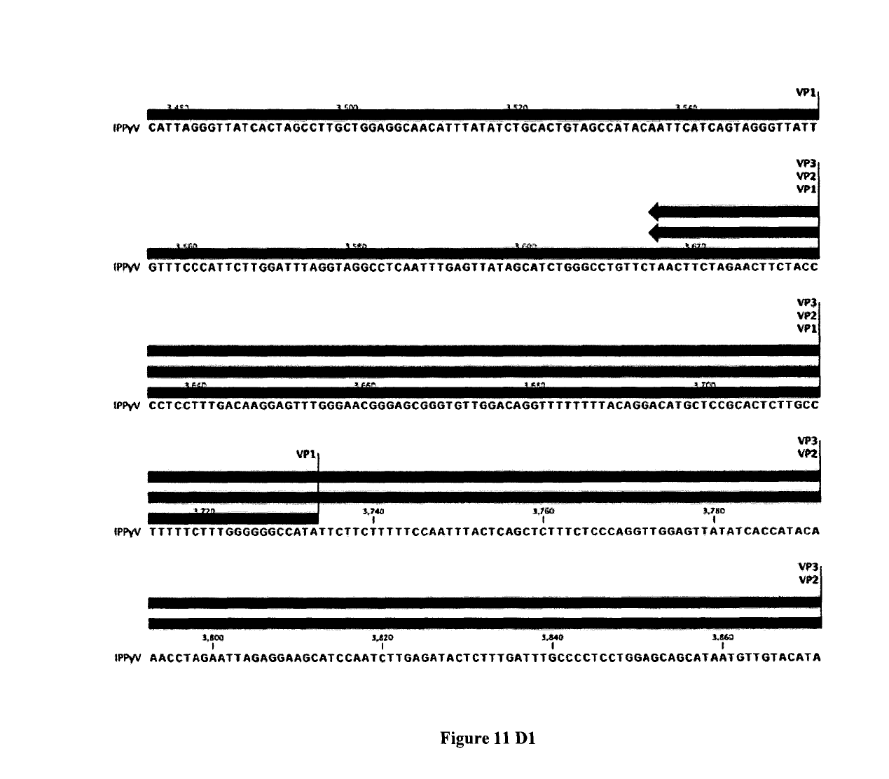

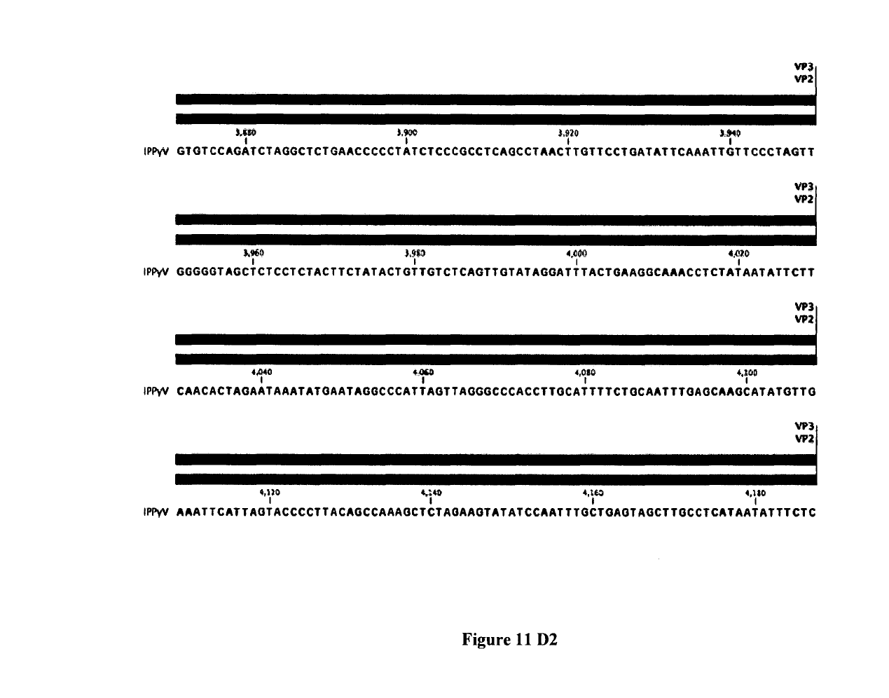

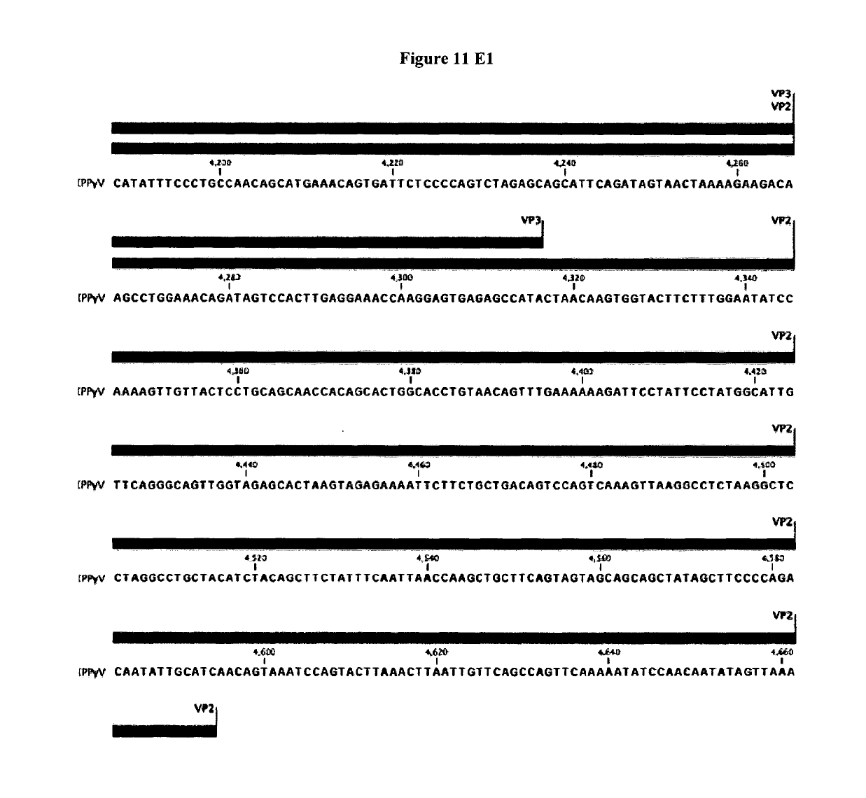

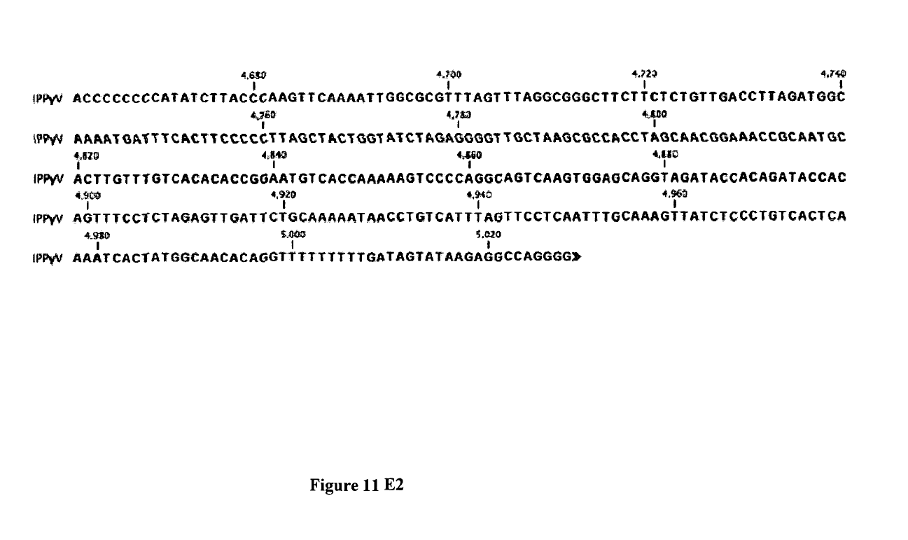

FIG. 11: Location and nucleotide sequence of the Open Reading Frames for Large T (LT), Small T, VP1, VP2 and VP3 proteins

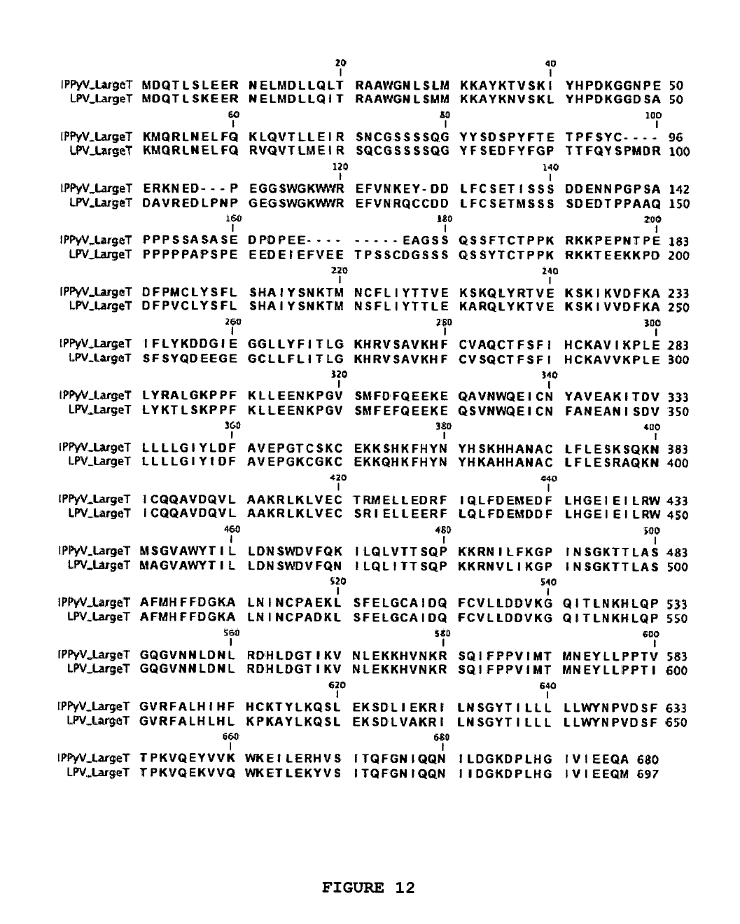

FIG. 12: Alignment of Large T amino acid sequence and LPV.

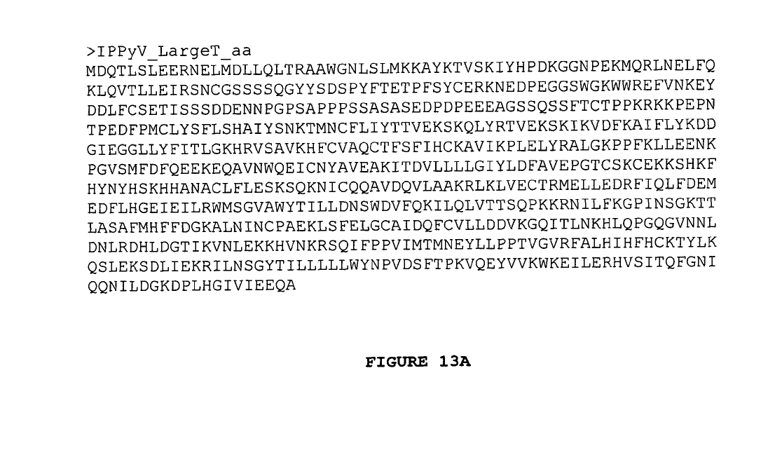

FIG. 13: Amino acid sequence and nucleotide sequence of large T (FIGS. 13A and 13B).

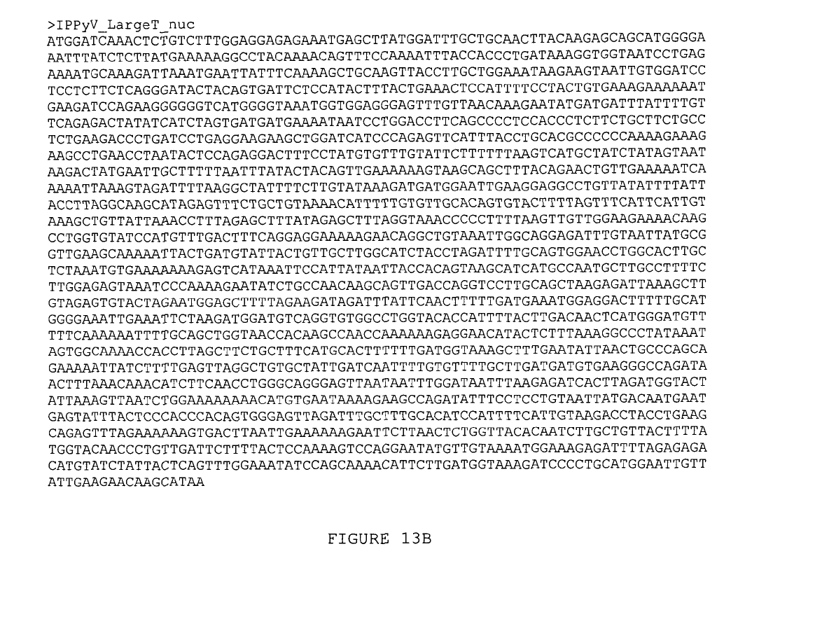

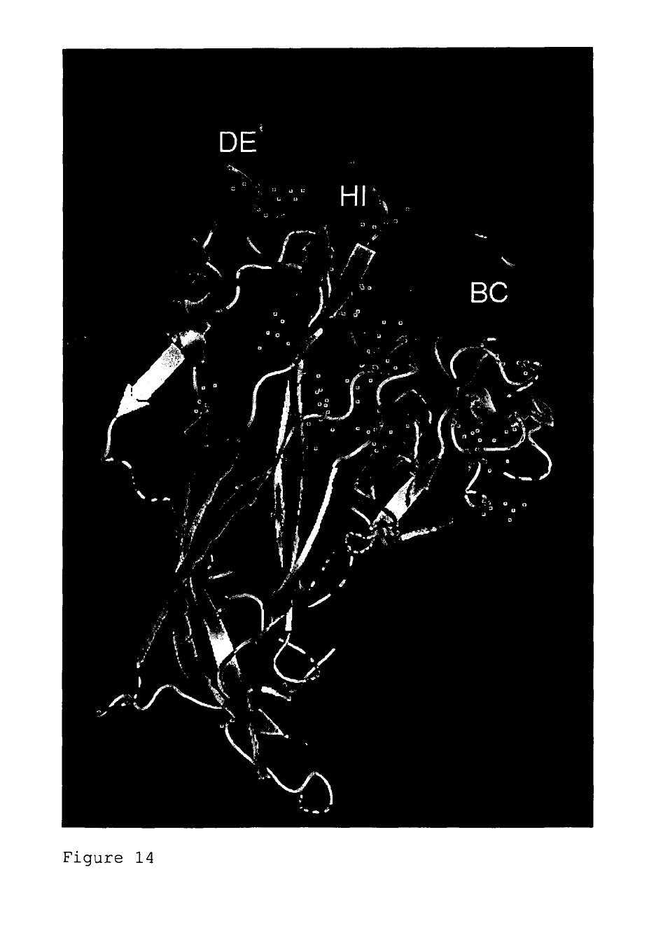

FIG. 14: Identification of VP1 residues. The BC, DE and HI loops that extend outward from VP1 are depicted. The crystal structure of SV40 VP1, derived from id 3BWQ, was used as template. Residues differing between IPPyV and LPV are depicted by pink dots.

### EXPERIMENTAL EXAMPLES

Identification of IpPyV

### Example 1

The inventors have screened DNA samples extracted with an automatic EasyMag apparatus (BioMérieux, Marcy l'Etoile, France) from cutaneous swabs taken from a patient (whose consent was dully obtained) suffering from a Merkel cell carcinoma (sample 100066) and from five healthy controls (samples 100067,100069, 100070, 100072 100073). The inventors have amplified these DNAs by the bacteriophage phi29 polymerase based multiple displacement amplification (MDA) assay using random primers. The ligation and WGA were performed essentially according to with the QuantiTect® Whole Transcriptome kit (Qiagen) according to the manufacturer's instructions. This provides concateners of high molecular weight DNA.

Sequencing was conducted by an Illumina High Seq sequencer. 5 μg of high molecular weight DNA resulting from isothermal amplification of each sample was fragmented into 200 to 350 nt fragments, to which were ligated adapters. 8 052 770 reads of 100 nt were derived from the sample from the diseased patient (100066), while respectively 10 354 496, 9 107 144, 8 196 240, 7 588 712, 10 281 130 reads were derived from the five healthy controls (samples 100067, 100069, 100070, 100072 100073).

Sorting out the flow of Illumina sequences was first done by a subtractive database comparison procedure. To this end, the whole host genome sequence (NCBI build 37.1/assembly hg19) was scanned with the reads using SOAPaligner (remaining from the diseased patient (100066): 3166256 reads; for the five controls: 5226679, 7979742, 2637289, 4664094, 5705 994 respectively). A quick and very restrictive BLASTN study was also performed to eliminate additional host reads (from the diseased patient (100066): 2894141 reads, for the five controls: 4625 257, 7734661, 1 621497, 4469 243, 5289856 respectively). The best parameters to be used have been determined previously. A number of assembly programs dedicated to short or medium-sized reads (Velvet, SOAPdenovo, CLC) have been tested for their efficiency in our pipeline. Optimal parameters have been set. The comparison of the single reads and contigs with already known genomic and taxonomic data was done on dedicated specialized viral, bacterial and generalist databases maintained locally (GenBank viral and bacterial databases, nr). The aforementioned databases were scanned using BLASTN and BLASTX. Binning (or taxonomic assignment) was based on the lowest common ancestor from the best hits among reads with a significant e-value.

Some reads from the diseased patient (100066) could be assembled into contigs homolog of different human viruses or bacteria. The inventors have more deeply analyzed 14 of them because they showed a better homology with a virus (NCBI accession: M30540, version M30540.1, GI:333282) of African green monkeys (AGM) than with any other virus, including other human or animal members of the Polyomaviridae family. Despite that AGM polyomavirus, also known “Monkey B-lymphotropic papovavirus” or as “lymphotropic polyomavirus” (LPV) has been described and isolated more than 30 years ago (2) from a lymphoblastoid cell line of AGM, no homolog virus has currently been described in humans. In fact, the presence of a virus close to the LPV in humans has been suspected since more than 30 years due to the presence of cross-reacting antibodies in a part of the human population without any contact with monkeys (3). Also PCR amplifications of short sequences matching to the LAV in human have been reported, but despite intensive research no sufficient genomic sequences for medical applications could be derived (4) (5). The inventors define here a new virus species we have named PIPyV (Pasteur Institute Polyomavirus), which characteristics are shown below, and the inventors provide sufficient sequence data for medical applications, including diagnostic and vaccine applications.

FIG. 1 depicts the mapping of the 14 contigs on the reference sequence of LPV (NCBI accession: M30540, version M30540.1, GI:333282) (NCBI Blastn). As can be seen, large regions of each coding gene have been sequenced. FIG. 2 shows the alignment of each contig with the homolog regions of LPV.

Based on the sequence of 8 of the contigs distributed along the genome, the inventors have defined a set of 9 primer pairs encompassing the whole target genome (Table 1).The primers allowed to amplify all the genome by PCR and sequence them by a conventional Sanger method. FIG. 3 shows the whole genome sequence of IPPyV.

Table 2 shows the localization of each putative open reading frame together with the characteristics of the corresponding proteins, which have been named according to their homology with other polyomaviruses proteins. Table 3 provides a comparison of the proteins of the IPPy virus with those of prior known polyomaviruses.

TABLE 1
Set of primers based on the sequence of the some of the contigs
Forward primers are in bold underlined and reverse primers are in italic
underlined. The sizes of the amplicons are given in base pairs (bp)

>Contig649
CTTCACATCATCAAGCAAAACACAAAATTGATCAATAGCACAGCCTAACTCAAAAGAT
AATTTTTCTGCTGGGCAGTTAATATTCAAAGCTTTACCATCAAAAAAGTGCATGAAAG
CAGAAGCTAAGGTGGTTTTGCCACTATTTATAGGGCCTTTAAAGAGTATGTTCCTCTT
TTTTGGTTGGCTTGTGGTTACCAGCTGCAAAATTTTTTGAAAAACATCCCATGAGTTG
TCAAGTAAAATG

| | | | | | |
|---|---|---|---|---|---|
| Forward | 28 | 60 | TGATCAATAGCACAGCCTAAC | 66.649.181F | 181 bp |
| Reverse | 208 | 60 | AATTTTGCAGCTGGTAACCAC | 66.649.181R | |

>Contig650
AATCCATATTTCTCATTAATAAGGTCCAGTAGTTAAAAGATGTAAAATCCTCAGGAAA
TCCAAACCAAAGTAAGTAGCACTTGTAGCAAAAGCATTCACCCCATACTAAGCAAGG
CTTTTTTTTATTTATTTTTGTACTTTTGTGCTGCTGCTTTAATAAACATACAATGCAGCA
ACAACTGGGCTTATT

| | | | | | |
|---|---|---|---|---|---|
| Forward | 10 | 60 | TCTCATTAATAAGGTCCAGTAG | 66.650.150F | 150 bp |
| Reverse | 159 | 60 | TATTAAAGCAGCAGCACAAAAG | 66.650.150R | |

>Contig816
AAGTAAAATGGTGTACCAGGCCACACCTGACATCCATCTTAGAATTTCAATTTCCCC
ATGCAAAAAGTCCTCCATTTCATCAAAAAGTTGAATAAATCTATCTTCTAAAAGCTCCA
TTCTAGTACACTCTACAAGCTTTAATCTCTTAGCTGCAAGGACCTGGTCAACTGCTTG
TTGGCAGATATTCTTTTGGGATTTACTCTCCAAGAAAAGGCAAGCATTGGCATGATGC
TTACTGTGGTAATTATAATGGAATTTATGACTCTTTTTTTCACATTTAGAGCAAGTGCC
AGGTTCCACTGCAAAATCTAGGTAGATGCCAAGCAACA

| | | | | | |
|---|---|---|---|---|---|
| Forward | 5 | 64 | AAATGGTGTACCAGGCCACAC | 66.816.303F | 303 bp |
| Reverse | 307 | 64 | TTTTGCAGTGGAACCTGGCAC | 66.816.303R | |

>Contig1378
ATTGAAGGAGGCCTGTTATATTTTATTACCTTAGGCAAGCATAGAGTTTCTGCTGTAA
AACATTTTTGTGTTGCACAGTGTACTTTTAGTTTCATTCATTGTAAAGCTGTTATTAAA
CCTTTAGAGCTTTATAGAGCTTTAGGTAAACCCCCTTTTAAGTTGTTGGAAGAAAACA
AGCCTGGTGTATCCATGT

| | | | | | |
|---|---|---|---|---|---|
| Forward | 27 | 66 | ACCTTAGGCAAGCATAGAGTTTC | 66.1378.165F | 165 bp |
| Reverse | 191 | 66 | ATGGATACACCAGGCTTGTTTTC | 66.1378.165R | |

>Contig4636
CTTCAGTGCCCTGATAAATTCTGACTTCTTCCACTTGCCCTGAAGTTCCTTCCATGGG
TTGTCCCTGAATTTGAGGCATAAGACCAGAGAACAAGCTATTAAGGAGAGAGCTTAC
AGGATAAGGATTTTTAACAACCCGTTTCCTTAGAGTTACATTAAAATATCTAGGTAGG
CCTCTCCAGTTTTGGGATTCTGAATAATTAGTGTGAATTCCAACAATATCAACAGCAG
ACAAAAACA

| | | | | | |
|---|---|---|---|---|---|
| Forward | 19 | 64 | TCTGACTTCTTCCACTTGCCC | 66.4636.171F | 171 bp |
| Reverse | 189 | 66 | CCCAAAACTGGAGAGGCCTAC | 66.4636.171R | |

>Contig6390
ATCTGAATGCTGCTCTAGACTGGGGAGAATCACTGTTTCATGCTGTTGGCAGGGAAA
TATGGAGAAATATTATGAGGCAAGCTACTCAGCAAATTGGATATACTTCTAGAGCTTT
GGCTGTAAGGGGTACTAATGAATTTCAACATATGCTTGCTCAAATTGCAGAAAATGCA
AGGTGGGCCCTAACTAATGGGCCTATTCATATTTATTCTAGTGTTGAAGAATATTATA
GAGGTTTGCCTTCAGTAAATCCTATACAACTGAGACAACAGTATAGAAGTAGAGGAG
AG

| | | | | | |
|---|---|---|---|---|---|
| Forward | 2 | 60 | CTGAATGCTGCTCTAGACTG | 66.6390.289F | 289 bp |
| Reverse | 290 | 60 | CTCTCCTCTACTTCTATACTG | 66.6390.289R | |

>Contig6671
AATTACTTCTTATTTCCAGCAAGGTAACTTGCAGCTTTTGAAATAATTCATTTAATCTT
TGCATTTTCTCAGGATTACCACCTTTATCAGGGTGGTAAATTTTGGAAACTGTTTTGTA
GGCCTTTTTCATAAGAGATAAATTTCCCCATGCTGCTCTTGTAAGTTGCAGCAAATCC
ATAAGCTCATTTCTCTCCTCCAAAGACAGAGTTTGATCCATGGCTCTGCAAAAAGTAA
AATAAGTCTTACTACCTGAGAATCAAGTTAATTAAGTTT

| | | | | | |
|---|---|---|---|---|---|
| Forward | 14 | 60 | TCCAGCAAGGTAACTTGCAG | 66.6671.195F | 195 bp |
| Reverse | 208 | 60 | ACTCTGTCTTTGGAGGAGAG | 66.6671.195R | |
| Forward | 0 | 64 | AATTACTTCTTATTTCCAGCAAGG | 66.6671.274F | 274 bp |
| Reverse | 273 | 64 | AAACTTAATTAACTTGATTCTCAGG | 66.6671.274R | |

>Contig7646
AAGTGTGTCACAGGTCATGTCTTCATTTAGCATGGGTAGCTTAATTACAGCCACTGA
GTAAGTAGGGAGGGTAGTAGCATTAGGGTTATCACTAGCCTTGCTGGAGGCAACATT
TATATCTGCACTGTAGCCATACAATTCATCAGTAGGGTTATTGTTTCCCATTCTTGGAT
TTAGGTAGGCCTCAATTTGAGTTATAGCATCTGGGCCTGTTCTAACTTCTAGAACTTC
TACCCCTCCTTTGACAAGGAG

| | | | | | |
|---|---|---|---|---|---|
| Forward | 46 | 64 | ACAGCCACTGAGTAAGTAGGG | 66.7646.206F | 206 bp |
| Reverse | 251 | 64 | TCCTTGTCAAAGGAGGGGTAG | 66.7646.206R | |

TABLE 2
Localization of ORF encoding IPPyV proteins and corresponding
encoded antigens.

| | | | Nbr of | |
|---|---|---|---|---|
| | Putative open | | amino | Theoretical |
| Protein | reading frame(s) | Frame | acids | mass kDA |
| VP1 | 3733-2618 | −1 | 371 | 40.33 |
| VP2 | 4673-3615 | −3 | 352 | 38.9 |
| VP3 | 4316-3615 | −3 | 233 | 26.95 |
| ST | 147-716 | +3 | 189 | 22.24 |
| LT | 147-383, 738-2114 | +3 | 537 | 61.24 |

TABLE 3
Comparison of amino acid sequence identity of IPPyV antigens,
with corresponding antigens in various polyomaviruses.

| IPPyV | JCV | BKV | KIV | WuV | MCyV | TSV | SV40 | LPV |
|---|---|---|---|---|---|---|---|---|
| VP1 | 53.9 | 53.2 | 28.3 | 28.3 | 54.8 | 60.6 | 52.9 | 87.1 |
| VP2 | 32.3 | 32.6 | 23.8 | 20.8 | 26.1 | 43.5 | 33.1 | 74.9 |
| VP3 | 34.1 | 35.9 | 24.5 | 20.3 | 15.1 | 41.3 | 33.7 | 72.5 |
| ST | 35.1 | 34 | 39.5 | 34.6 | 40.1 | 42.5 | 31.8 | 81 |
| LT | 29.9 | 30.4 | 33 | 31.5 | 30.8 | 37.8 | 30.4 | 61.3 |

Protein VP1.

VP1 is a capsid protein, which is important for tropism as it mediates interactions with the cell receptor(s). For non-enveloped viruses as Polyomaviridae, it is also a major target of antibodies, including neutralizing and opsonising antibodies and thus is fundamental for medical application as it is the main candidate for the development of vaccines and serological tests.

FIG. 4 shows the sequence of VP1 translated from its nucleotide sequence, which location was deduced by its homology with the LPV and other viruses of the Polyomaviridae family. Nucleotidic and aminoacid alignments with the closest known VP1 from the LPV is shown in FIG. 5. The nucleotic and aminoacid identity between these two proteins are respectively and 87.1%.

Alignment of this protein with other representatives of the human and animal Polyomaviridae family by MUSCLE using curation by G-Blocks allowed to generate a phylogenetic tree using PhymL (Phylogeny package) (FIG. 6). This shows that the primate viruses closest to IPPyV are the LPV, and, more distant the human oncogenic Merkel cell polyomavirus (MCPyV) the TSPyV and the chimpanzee virus.

The crystal structure of the all VP1 of LPV and MCPyV is not known, but their VP1 polypeptide harbour BC, DE, EF, GH, and HI domains, determined based on the homology with other polyomaviruses. In the well studied JC virus, mutations in the loops determine tropism and prognostic of the disease (7). Also, the receptor binding site of BKV seems formed by the BC and HI loops (8). The BC loop of BKV is polymorph and allows to define four viral subtypes, which also correspond to four viral antigenic subtypes (9) (10). The four BKV subtypes have a different geographical distribution. It was thus important to compare the loops of IPPyV to those of the closer polyomaviruses. FIG. 7 compares the BC, DE and HI loops of IPPyV to the two closer primate polyomaviruses LPV and MCPyV. As shown in FIG. 7, the BC, DE and HI loops are closer (but different) between LPV and PIPyV than compared to MCPyV. It has been reported than antibodies against LPV are found in 6 to 23% of the human population in function of the age, but that these antibodies do not cross react with other polyomaviruses (11), suggesting that a polyomavirus close to LPV circulates in humans. According to the inventors, the IPPyV might be such a virus. Furthermore, differences between VP1 loops implicate that use of antigens including one or several of the IPPyV BC, DE and HI loops will permit to prepare more specific and sensible antibody test than by using the LPV heterologous antigen. Also, immunogenic compositions and vaccines based on the true sequence of VP1 and more specifically its immunogenic loop domains can be derived from the sequence.

T Antigens

A striking feature of the virus is the presence in the sequenced clone of a mutation that introduces a stop codon in the large T antigen. Alignment of the large T antigens between IPPyV and LPV is shown FIG. 9. The stop codon is located in such a way that it keeps the transforming properties of the large T antigen but impairs the expression of the large T COOH-terminus, which includes the helicase function. This is a property shared with some oncogenic strains of polyomaviruses, including the Merkel polyomavirus (12). This result also strongly suggests that others isolates harbouring a full large T protein, allowing for efficient replication of the virus should exist in the human population, including in the same patient (13).

Association with Disease

Based on the knowledge of the sequence of IPPyV, the inventors have screened if reads from the skin of five healthy controls matched with this new virus. Among the 4625 257, 7734661, 1621497, 4469 243, 5289856 non-human reads generated respectively from samples 100067, 100069, 100070, 100072 100073, none was similar to this virus genome. The inventors have also screened more than 10⁶ reads from plasma samples from 14 people without known evoluting cancer. None of these reads matched with the IPPyV genome. On the other hand, all the six skin samples, including the diseased patient (who was developing a Merkel carcinoma) and the five healthy controls, were infected by the MCPyV, which could be evidenced at the skin surface by PCR. They have sequenced the whole genome of MCPyV coming from the skin swabs of these 6 patients and did not find a specific mutation associated with the disease. Thus, in this patient, the presence of IPPyV was more strongly associated with the disease (Merkel carcinoma) than was the presence of MCPyV. Furthermore this strain harbours a stop codon within the large T antigen that is common feature of oncogenic polyomaviruses genomes. Thus IPPyV could be a factor or a cofactor contributing to the venue and/or the prognostic of Merkel carcinoma and other cancers.

### Example 2

Methods

Subjects and Sample Collection

For analysis by HTS, six DNA samples extracted from cutaneous swabs obtained from the face across the forehead and eyebrows of patients previously studied by PCR for the presence of MCPyV sequences were selected (Foulongne et al).

These samples were obtained by swabbing the skin of an index patient with MCC away from the tumor lesion, and the skin of 5 healthy individuals.

For investigation of prevalence by specific nested-PCR assay, 120 apparently healthy skin specimens were similarly collected from volunteer subjects. The median age was 48 years (range 19-96) and the 30 older persons were 57-96 years old, with a median age of 71 years.

These volunteers were 40 patients hospitalized or attending outpatient clinics at the dermatology unit of the Montpellier University hospital for various skin disorders (including 8 patients with MCC, median age 75 (range 57-86), 20 immune-compromised patients without skin lesions (divided in 10 patients infected with HIV-1 without skin symptoms and 10 renal transplant recipients under immunosuppressive regimens that usually associate steroid, mycophenolate mofetil and calcineurin inhibitors), and 60 healthy control subjects. Respiratory samples were 46 bronchoalveolar lavage (BAL) samples obtained from hospitalized patients in intensive care units with acute respiratory failure of unknown origin and 46 nasopharyngeal aspirates (NPA) from children attending the pediatric emergency unit of the Montpellier university hospital for various respiratory tract disorders. An additional set of 92 stools samples were collected from children hospitalized in the pediatric unit for gastroenteritis.

Extraction and Amplification of DNA

DNA from all samples was extracted as previously described (Foulongne et al) For HTS, DNA was amplified by a bacteriophage phi29 polymerase-based rolling circle amplification assay using random primers. The protocol of the QIAGEN REPLI-g Midi Kit (Qiagen, Courtaboeuf, France) was followed as recommended by the manufacturer.

High Throughput Sequencing

HTS, based on Illumina HiSeq 2000 apparatus, was performed by GATC Biotech AG (Konstanz, Germany). Five pg of high molecular weight DNA resulting from amplification was fragmented into 200 to 350 nt fragments, to which were ligated adapters including a nucleotidic tag allowing for multiplexing several samples per lane or channel. Sequencing was conducted with a mean depth per sample of 8.9×10⁶ paired-end reads of 100 nt in length (range 7.6-10.3×10⁶).

Sequence Analysis

Sequences were first sorted by a subtractive database comparison procedure, and a number of assembly programs dedicated to short or medium-sized reads were used to generate contigs (Velvet (http://www.ebi.ac.uk/˜zerbino/velvet/), SOAPdenovo (http://soap.genomics.org.cn/), CLC Genomics Workbench (http://www.clcbio.com)) as previously described (Cheval et al, submitted). The comparison of the single reads and contigs to already known genomic and taxonomic data was performed on dedicated specialized viral, bacterial and generalist databases created and maintained at the Institut Pasteur (GenBank viral and bacterial databases, nr). The aforementioned databases were screened using BLASTN and BLASTX. We used Paracel BLAST (Pasadena, Calif.), software capable of executing searches on multiple non-shared memory processors simultaneously. The entire sequence of the IPPyV (Institut Pasteur Polyomavirus) strain genome was analyzed and annotated with CLC Genomics Workbench (CLC bio, Aarhus N, Denmark). The reference sequences of the other members of the Polyomaviridae family are JCPyV (NC_{—}001699), BKPyV (NC_{—}001538), KIPyV (NC_{—}009238), WuPyV (NC_{—}009539), MCPyV (NC_{—}010277), SV40 (NC_{—}001669), TSPyV (NC_{—}014361), LPV (M30540). Protein structures were visualized by Pymol (Delano Scientific LLC, San Francisco).

PCR

For sequencing the new polyomavirus genome IPPyV by the Sanger method, 9 primer pairs were designed so as to amplify the entire genome, by reference to the contigs assembled from HTS data acquired in the first phase (see results). Once the genome was sequenced, the inventors developed a specific nested PCR for the detection of IPPyV in samples, using primers based on the IPPyV genome sequence and designed using PrimerPro 3.4 software (www.changbioscience.com/) as follows: VP1_{—}354F

ID N° 85 (5′_ACCATATCAGTAGGATAGGTA_{—}3′) and VP1_{—}354R

ID N° 86 (5′_TGAATTGTATGGCTACAGTGC_{—}3′) for the outer PCR and VP1_{—}198F

ID N° 87 (5′_CACTGGGATAGTTCCTGAGG_{—}3′) and VP1_{—}198R

ID N° 88 (5′_CCTAATGCTACTACCCTCCCT_{—}3′) for the inner PCR. These primers were designed so as to avoid amplification of other known human polyomaviruses.

Statistical Analysis

Confidence intervals for proportions were calculated according to the efficient-score method (corrected for continuity) (Newcombe et al) (http://dogsbody.psych.mun.ca/VassarStats/).

Ethical Approval

The studies were approved by the Institut Pasteur “Comité de Recherche Clinique” and the French “Commission Nationale Informatique et Libertés” (09.465). Consent was sought for human samples according to French regulations.

Results

Identification of the IPPyV Atrain

Among the 8,052,770 Illumina reads obtained from the DNA extracted from the skin surface of the MCC index patient, the inventors were able to assemble the complete genome of MCPyV (not shown). They also found numerous papillomavirus contigs, together with contigs covering more than half of the genome of HPyV6 and HPyV7 (not shown). Additionally, 14 other contigs were assembled that showed a better homology with LPV (NCBI accession: M30540, version M30540.1, GI:333282) than with any other virus present at that time in the NCBI nr database, including other human or animal members of the Polyomaviridae family. Based on the sequence of the 8 of the 14 obtained contigs, which were distributed along the LPV genome, the inventors defined a set of 9 primer pairs encompassing the whole target genome. These primers allowed amplification of the entire genome by PCR and analysis of its sequence of 5,028 nt by the Sanger method, which confirmed its circular nature. It encodes analogs of small and large T antigens, and structural proteins VP1, VP2 and VP3, and does not appear to encode an agnoprotein. The pairwise amino-acid identity between IPPyV is in the range of 72-80% for LPV, and much lower with other known Polyomaviridae family members, as shown in Table 1.

Detection of IPPyV in Human Samples

The inventors first confirmed by a specific nested PCR the presence of IPPyV in the skin swab of the index case in which the virus had been identified by HTS. They also confirmed the presence of the virus by nested PCR in a second cutaneous sample of the same index case obtained 20 months after the first sampling. Because IPPyV was identified in a patient suffering from Merkel carcinoma (MCC), the inventors explored the skin surface of 7 other MCC cases. IPPyV was detected in one other case, an 80 year-old patient. The overall prevalence in the MCC group was thus 2/8 [25% (4.4-64%, p=0.05)]. Because the inventors were interested in the possibility of inter-human transmission of IPPyV, a skin swab from the wife of the first index case was tested: it was positive.

The inventors sampled 111 skin swabs from healthy persons or non-MCC patients without any known contact with MCC cases and screened them with the same nested-PCR assay. Only one healthy 30-year-old individual harbored IPPyV, demonstrating a low prevalence in this control group of 1/111 [0.9% (0.05-5.6%, p=0.05)]. The considered MCC population was 57-86 years old (median: 75), and as the inventors were unable to detect IPPyV among the 30 older controls (57-96 years, median 71 years), this suggests that the rate of detection in the MCC samples was not biased by the older ages of these patients. None of the 92 respiratory and 92 stool specimens were positive for IPPyV.

Discussion

There is now much evidence that healthy human skin harbors numerous viruses. This has been extensively described for cutaneous human papillomavirus (HPV) that are commonly present on the superficial layers of the skin of most individuals (Feltkamp et al). The recent description of new human viruses belonging to the Polyomaviridae family suggests that some of these viruses share the cutaneous tropism of β- and gamma-HPV. Merkel cell polyomavirus (MCPyV) associated with MCC is also detected at the surface of healthy skin in the majority of the population (Foulongne et al) (Wieland et al), and two additional representatives of the human polyomavirus (HPyV) genus, named HPyV6 and HPyV7, have been identified as well at the surface of the skin of healthy patients (Schowalter et al). Detection of an additional human polyomavirus in cutaneous samples reinforces the perception of the skin as a complex micro-ecosystem colonized by many different viruses, with polyomaviruses representing part of this viral microbiota. The existence in the human population of a polyomavirus closely related to LPV, whose natural host is the African green monkey, has been anticipated (3). In fact, an African green monkey polyomavirus, also known as “Monkey B-lymphotropic papovavirus” or “lymphotropic polyomavirus” (LPV), was isolated more than 30 years ago (2) from a lymphoblastoid cell line derived from the African green monkey. The presence of a virus closely related to LPV has been suspected for over 30 years owing to the presence of cross-reacting antibodies in human subjects without any known contact with monkeys (3)((11)). PCR amplification of short sequences matching those of the LPV has been reported, but the length and overlap of these sequences were insufficient for characterizing this elusive virus (4) (5) show here that the sequence of this virus makes it a good candidate to be the target of antibodies previously found in humans.

The chronic shedding of HPyVs from skin surface is reminiscent of a well-known feature of cutaneous HPVs that replicate in keratinocytes and are likely to be transmitted environmentally or through interindividual contact. Strikingly, in the inventor's study, the detection of IPPyV in the skin of the index case's wife suggests a similar route of transmission.

It has been proposed that MCPyV and HPyV6 or HPyV7 may infect superficial cells of the epidermis and that production of virions may be, as for HPVs, linked to the differentiation of the epidermis (Schowalter et al). The inventors cannot rule out a similar scenario for IPPyV. However, since its closest relative (LPV) has been described, based on its in vitro growth ability, as a lymphotropic virus, the ability of IPPyV to infect lymphoid precursors is worth considering as well as its putative role in various lymphoproliferative disorders in humans.

IPPyV was detected in cutaneous samples but not in respiratory and stool samples, and the rate of detection appears lower than that previously reported for MCPyV (review in (Agelli et al)) or HPyV6-7 as well (Schowalter et al). The sampling site was chosen regarding methods previously used for the detection of HPyVs on the skin (Schowalter et al) (Wieland et al). Furthermore, we have previously noticed a particular pattern for MCPyV shedding since face swabs yielded a higher rate of viral detection than limbs (Foulongne et al). However, since HPyVs shedding was not as extensively studied than that of HPVs, the inventors cannot rule out a similar pattern of excretion leading to underestimate the detection of IPPyV on unique face swabs. The exact prevalence of IPPyV should be investigated through serological and PCR assays, notably to investigate the significance of previously published data suggesting that around 30% of humans present antibodies that recognized a LPV-like virus.

Since clinical manifestations associated with HPyVs infections dramatically increase in immune-compromised patients, clinical manifestations caused by IPPyV, should they exist, are also more likely to occur in this category of patients. Furthermore, IPPyV infection might not remain restricted to the cutaneous compartment in immune-compromised patients, and IPPyV reactivation might lead to a systemic dissemination and in some cases result in clinical symptoms.

TABLE 1
Amino acid identity between putative proteins encoded by IPPyV and the proteins of selected members of
Polyomaviridae deduced from pairwise sequence alignment (EMBOSS, Needle software).

| | putative open reading | | # amino | Amino acid identity (%) | | | | | | | |
|---|---|---|---|---|---|---|---|---|---|---|---|
| Protein | frame (s) | frame | acids | JCV | BKV | KIV | WuV | MCyV | TSV | SV40 | LPV |
| VP1 | 1443-2558 | +3 | 371 | 53.9 | 53.2 | 28.3 | 28.3 | 54.8 | 60.6 | 52.9 | 87.1 |
| VP2 | 503-1561 | +2 | 352 | 32.3 | 32.6 | 23.8 | 20.8 | 26.1 | 43.5 | 33.1 | 74.9 |
| VP3 | 860-1561 | +2 | 233 | 34.1 | 35.9 | 24.5 | 20.3 | 15.1 | 41.3 | 33.7 | 72.5 |
| ST antigen | 5028-4459 | −1 | 189 | 35.1 | 34 | 39.5 | 34.6 | 40.1 | 42.5 | 31.8 | 81 |
| LT antigen | 5028-4792, 4437-2632 | −1 | 680 | 40.4 | 41.2 | 44.2 | 42 | 39.9 | 49.3 | 40 | 80.5 |

### CITED REFERENCES

(1) van der Meijden E, Janssens R W, Lauber C, Bouwes Bavinck J N, Gorbalenya A E, Feltkamp M C. Discovery of a new human polyomavirus associated with trichodysplasia spinulosa in an immunocompromized patient. PLoS Pathog. 2010 Jul. 29; 6(7)

(2) zur Hausen H, Gissmann L. Lymphotropic papovaviruses isolated from African green monkey and human cells. Med Microbiol Immunol. 1979 August; 167(3):137-53.

(3) Brade L, Müller-Lantzsch N, zur Hausen H. J Med Virol. B-lymphotropic papovavirus and possibility of infections in humans.1981; 6(4):301-8.

(4) Delbue S, Tremolada S, Branchetti E, Elia F, Gualco E, Marchioni E, Maserati R, Ferrante P First identification and molecular characterization of lymphotropic polyomavirus in peripheral blood from patients with leukoencephalopathies. J Clin Microbiol. 2008 July; 46(7):2461-2.

(5) Delbue S, Tremolada S, Elia F, Carloni C, Amico S, Tavazzi E, Marchioni E, Novati S, Maserati R, Ferrante Lymphotropic polyomavirus is detected in peripheral blood from immunocompromised and healthy subjects. J Clin Virol. 2010 February; 47(2):156-60

(6) J. P. Griffith, D. L. Griffith, I. Rayment, W. T. Murakami and D. L. Caspar, Inside polyomavirus at 25-A resolution, Nature 355 (1992), pp. 652-654

(7) Delbue S, Branchetti E, Bertolacci S, Tavazzi E, Marchioni E, Maserati R, Minnucci G, Tremolada S, Vago G, Ferrante P. J JC virus VP1 loop-specific polymorphisms are associated with favorable prognosis for progressive multifocal leukoencephalopathy Neurovirol. 2009 January; 15(1):51-6.

(8) A. Dugan, M. L. Gasparovic, N. Tsomaia, D. F. Mierke, B. A. O'Hara, K. Manley and W. J. Atwood, Identification of amino acid residues in BK virus VP1 critical for viability and growth, J. Virol. 81 (2007), pp. 11798-11808

(9) L. Jin, P. E. Gibson, W. A. Knowles and J. P. Clewley, BK virus antigenic variants: sequence analysis within the capsid VP1 epitope, J. Med. Virol. 39 (1993), pp. 50-56

(10) W. A. Knowles, P. E. Gibson and S. D. Gardner, Serological typing scheme for BK-like isolates of human polyomavirus, J. Med. Virol. 28 (1989), pp. 118-123.

(11) Kean J M, Rao S, Wang M, Garcea R L. Seroepidemiology of human polyomaviruses. PLoS Pathog. 2009 March; 5(3)

(12) Shuda M, Feng H, Kwun H J, Rosen S T, Gjoerup O, Moore P S, Chang Y. T antigen mutations are a human tumor-specific signature for Merkel cell polyomavirus. Proc Natl Acad Sci USA. 2008 Oct. 21; 105(42):16272-7

(13) Laude H C, Jonchère B, Maubec E, Carlotti A, Marinho E, Couturaud B, Peter M, Sastre-Garau X, Avril M F, Dupin N, Rozenberg F Distinct merkel cell polyomavirus molecular features in tumour and non tumour specimens from patients with merkel cell carcinoma. PLoS Pathog. 2010 Aug. 26; 6(8). 1001076.

(14). Schowalter R. M., D. V. Pastrana, K. A. Pumphrey, A. L. Moyer, et C. B. Buck. 2010. Merkel cell polyomavirus and two previously unknown polyomaviruses are chronically shed from human skin. Cell Host Microbe 7:509-515.

(15). Agelli M., L. X. Clegg, J. C. Becker, et D. E. Rollison. 2010. The etiology and epidemiology of merkel cell carcinoma. Curr Probl Cancer 34:14-37.

(16). Feltkamp M. C. W., M. N. C. de Koning, J. N. B. Bavinck, et J. Ter Schegget. 2008. Betapapillomaviruses: innocent bystanders or causes of skin cancer. J. Clin. Virol 43:353-360.

(17). Feng H., M. Shuda, Y. Chang, et P. S. Moore. 2008. Clonal integration of a polyomavirus in human Merkel cell carcinoma. Science 319:1096-1100.

(18). Foulongne V., N. Kluger, O. Dereure, G. Mercier, J. P. Moles, B. Guillot, et M. Segondy. 2010. Merkel cell polyomavirus in cutaneous swabs. Emerging Infect. Dis 16:685-687.

(19). Johnson E. M. 2010. Structural evaluation of new human polyomaviruses provides clues to pathobiology. Trends Microbiol 18:215-223.

(20). Newcombe R. G. 1998. Two-sided confidence intervals for the single proportion: comparison of seven methods. Stat Med 17:857-872.

(21) Wieland U., C. Mauch, A. Kreuter, T. Krieg, et H. Pfister. 2009. Merkel cell polyomavirus DNA in persons without merkel cell carcinoma. Emerging Infect. Dis 15:1496-1498.

1-29. (canceled)

30. A recombinant polynucleotide or non naturally occurring polynucleotide that comprises or consists of:
A) the sequence shown in SEQ ID NO: 1 or in SEQ ID NO: 82;
B) an inverse complement of the sequence shown in SEQ ID NO: 1 or in SEQ ID NO: 82;
C) a fragment of at least 50 nucleotides of the sequence of A) or B); or
D) a sequence that hybridizes under stringent conditions with at least one of A), B), and C).

31. The polynucleotide as claimed in claim 30, comprising or consisting of a sequence selected from:
A) the sequence of SEQ ID NO: 2 (VP1) or a sequence at least 44.9% identical thereto;
B) the sequence of SEQ ID NO: 4 (VP2) or a sequence at least 77.5% identical thereto;
C) the sequence of SEQ ID NO: 6 (VP3) or a sequence at least 75.5% identical thereto;
D) the sequence of SEQ ID NO: 83 (Large T) or a sequence at least 60.2% identical thereto; or
E) the sequence of SEQ ID NO: 10 (Small T) or a sequence at least 78.6% identical thereto.

32. The polynucleotide as claimed in claim 30, comprising or consisting of a sequence selected from:
A) the sequence of SEQ ID NO: 12;
B) the sequence of SEQ ID NO: 14;
C) the sequence of SEQ ID NO: 16;
D) the sequence of SEQ ID NO: 85;
E) the sequence of SEQ ID NO: 86;
F) the sequence of SEQ ID NO: 87; or
G) the sequence of SEQ ID NO: 88.

33. A vector comprising a polynucleotide as claimed in claim 30.

34. A recombinant polypeptide of at least 20 amino acids comprising a polypeptide sequence encoded by a polynucleotide that:
A) is the sequence shown in SEQ ID NO: 1 or in SEQ ID NO: 82;
B) is an inverse complement of the sequence shown in SEQ ID NO: 1 or in SEQ ID NO: 82;
C) is a fragment of at least 60 nucleotides of the sequence of A) or B); or
D) is a sequence that hybridizes under stringent conditions with at least one of A), B), and C).

35. The recombinant polypeptide as claimed in claim 34, that is encoded by a polynucleotide sequence that is:
A) the sequence of SEQ ID NO: 2 (VP1) or a sequence at least 44.9% identical thereto;
B) the sequence of SEQ ID NO: 4 (VP2) or a sequence at least 77.5% identical thereto;
C) the sequence of SEQ ID NO: 6 (VP3) or a sequence at least 75.5% identical thereto;
D) the sequence of SEQ ID NO: 83 (Large T) or a sequence at least 60.2% identical thereto; or
E) the sequence of SEQ ID NO: 10 (Small T) or a sequence at least 78.6% identical thereto.

36. The recombinant polypeptide as claimed in claim 34, that is:
A) a polypeptide comprising the amino acid sequence of SEQ ID NO: 3, SEQ ID NO: 5, SEQ ID NO: 7, SEQ ID NO: 84, SEQ ID NO: 11, SEQ ID NO: 13, SEQ ID NO: 15, or SEQ ID NO: 17;
B) a polypeptide that immunologically reacts with antibodies raised against a polypeptide comprising the amino acid sequence of SEQ ID NO: 3, SEQ ID NO: 5, SEQ ID NO: 7, SEQ ID NO: 84, SEQ ID NO: 11, SEQ ID NO: 13, SEQ ID NO: 15, or SEQ ID NO: 17; or
C) a polypeptide that is at least 70% identical to the amino acid sequence of SEQ ID NO: 3, SEQ ID NO: 5, SEQ ID NO: 7, SEQ ID NO: 84, SEQ ID NO: 11, SEQ ID NO: 13, SEQ ID NO: 15, or SEQ ID NO: 17.

37. An isolated antibody that binds to a polypeptide as claimed in claim 34, wherein the antibody does not cross-react with the following human polyomaviruses: the JC virus, the BK virus, the KI polyomavirus, the WU polyomavirus, the Merkel cell polyomavirus, and the Trichodysphasia spinulosia-associated polyomavirus.

38. A kit for detecting a polyomavirus of the IPPyV species, comprising a recombinant polypeptide as claimed in claim 34 and an immunological detection reagent.

39. A kit for detecting a polyomavirus of the IPPyV species, comprising an antibody as claimed in claim 37 and an immunological detection reagent.

40. A method for detecting antibodies against a polyomavirus of the IPPyV species, comprising:
A) providing a sample;
B) contacting the sample with a polypeptide as claimed in claim 34; and
C) assaying for an immunological reaction between an antibody in the sample and the polypeptide.

41. A method for detecting an antigen of a polyomavirus of the IPPyV species, comprising:
A) providing a sample;
B) contacting the sample with an antibody as claimed in claim 37; and
C) assaying for an immunological reaction between the sample and the antibody.

42. A method for detecting an IPPyV nucleic acid sequence, comprising:
A) providing a sample;
B) amplifying a IPPyV nucleic acid sequence in the sample; and
C) detecting the amplified IPPyV nucleic acid sequence.

43. The method of claim 42, wherein the method comprises contacting the sample with a probe or primer that specifically binds to a target sequence selected from the sequence shown in SEQ ID NO: 1 and the inverse complement of the sequence shown in SEQ ID NO: 1 or in SEQ ID NO: 82.

44. The method of claim 42, wherein the method comprises detecting hybridization between a probe and the amplified IPPyV nucleic acid sequence.

45. The method of claim 42, wherein the method comprises detecting amplification of the amplified IPPyV nucleic acid sequence.

46. The method of claim 42, wherein the method comprises sequencing the amplified IPPyV nucleic acid sequence.

47. The method of claim 42, wherein the method comprises amplifying the IPPyV nucleic acid sequence by PCR.

48. The method of claim 43, wherein the probe or primer is the polynucleotide show in SEQ ID NO: 85, SEQ ID NO: 86, SEQ ID NO: 87, and SEQ ID NO: 88.

49. The method of claim 47, wherein the PCR is performed with the pair of primers shown in SEQ ID NO: 85 and SEQ ID NO: 86.

50. The method of claim 47, wherein the PCR is performed with the pair of primers shown in SEQ ID NO: 87 and SEQ ID NO: 88.
